(19) United States
(12) Patent Application Publication
(10) Pub. No.: US 2011/0244025 A1
(43) Pub. Date: Oct. 6, 2011

(54) MODIFIED OLIGORIBONUCLEOTIDE ANALOGS WITH ENHANCED IMMUNOSTIMULATORY ACTIVITY

(76) Inventors: Eugen Uhlmann, Glashutten (DE); Arthur M. Krieg, Wellesley, MA (US); Grayson B. Lipford, Watertown, MA (US)

(21) Appl. No.: 12/946,379

(22) Filed: Nov. 15, 2010

Related U.S. Application Data
(63) Continuation of application No. 11/411,975, filed on Apr. 26, 2006, now abandoned.
(60) Provisional application No. 60/674,896, filed on Apr. 26, 2005.

(51) Int. Cl. A61K 9/127; C07H 21/02; A61K 39/00; A61P 11/00; A61P 31/14; A61P 35/00; A61P 37/08; A61P 37/04; A61P 11/06; C12N 5/02; B82Y 5/00

(52) U.S. Cl. 424/450; 536/23.1; 424/184.1; 424/277.1; 435/375; 977/700

(57) ABSTRACT

The invention provides immunostimulatory compositions and methods for their use. In particular, the immunostimulatory compositions of the invention include RNA-like polymers that incorporate an immunostimulatory sequence motif and at least one chemical modification to confer improved stability against nuclease degradation and improved activity. Specific modifications involving phosphate linkages, nucleotide analogs, and combinations thereof are provided. Compositions of the invention optionally include an antigen and can be used to stimulate an immune response. Also provided are compositions and methods useful for treating a subject having an infection, a cancer, an to allergic condition, or asthma. Modified oligoribonucleotide analogs of the invention are believed to stimulate Toll-like receptors TLR7 and TLR8.

### RELATED APPLICATION

This application claims benefit under 35 U.S.C. 119(e) of U.S. Provisional Application No. 60/674,896, filed Apr. 26, 2005, the entire contents of which is incorporated herein by reference.

### SEQUENCE LISTING

The entire contents of the compact disc containing the Sequence Listing identified as “C1041.70045US01 seq.txt”, recorded on Apr. 25, 2006, and containing 1.6 MB, is incorporated herein by reference.

### FIELD OF THE INVENTION

The invention relates generally to the field of immunology, and more particularly to immunostimulatory molecules. More specifically the invention relates to modified forms of ribonucleic acid (RNA) and RNA analogs with enhanced immunostimulatory activity compared to natural RNA.

### BACKGROUND OF THE INVENTION

Toll-like receptors (TLRs) are a family of highly conserved pattern recognition receptor (PRR) polypeptides that recognize pathogen-associated molecular patterns (PAMPs) and play a critical role in innate immunity in mammals. Currently at least ten family members, designated TLR1-TLR10, have been identified. The cytoplasmic domains of the various TLRs are characterized by a Toll-interleukin 1 receptor (TIR) domain. Medzhitov R et al. (1998) Mol Cell 2:253-8. Recognition of microbial invasion by TLRs triggers activation of a signaling cascade that is evolutionarily conserved in Drosophila and mammals. The TIR domain-containing adapter protein MyD88 has been reported to associate with TLRs and to recruit interleukin 1 receptor-associated kinase (IRAK) and tumor necrosis factor (TNF) receptor-associated factor 6 (TRAF6) to the TLRs. The MyD88-dependent signaling pathway is believed to lead to activation of NF-κB transcription factors and c-Jun NH₂ terminal kinase (Jnk) mitogen-activated protein kinases (MAPKs), critical steps in immune activation and production of inflammatory cytokines. For reviews, see Aderem A et al. (2000) Nature 406:782-87, and Akira S et al. (2004) Nat Rev Immunol 4:499-511.

A number of specific TLR ligands have been identified. Ligands for TLR2 include peptidoglycan and lipopeptides. Yoshimura A et al. (1999) J Immunol 163:1-5; Yoshimura A et al. (1999) J Immunol 163:1-5; Aliprantis A O et al. (1999) Science 285:736-9. Lipopolysaccharide (LPS) is a ligand for TLR4. Poltorak A et al. (1998) Science 282:2085-8; Hoshino K et al. (1999) J Immunol 162:3749-52. Bacterial flagellin is a ligand for TLR5. Hayashi F et al. (2001) Nature 410:1099-1103. Peptidoglycan has been reported to be a ligand not only for TLR2 but also for TLR6. Ozinsky A et al. (2000) Proc Natl Acad Sci USA 97:13766-71; Takeuchi O et al. (2001) Int Immunol 13:933-40. Recently certain low molecular weight synthetic compounds, the imidazoquinolines imiquimod (R-837) and resiquimod (R-848), were reported to be ligands of TLR7 and TLR8. Hemmi H et al. (2002) Nat Immunol 3:196-200; Jurk M et al. (2002) Nat Immunol 3:499.

Beginning with the recent discovery that unmethylated bacterial DNA and synthetic analogs thereof (CpG DNA) are ligands for TLR9 (Hemmi H et al. (2000) Nature 408:740-5; Bauer S et al. (2001) Proc Natl Acad Sci USA 98, 9237-42), it has been reported that ligands for certain TLRs include certain nucleic acid molecules. Recently it has been reported that certain types of RNA are immunostimulatory in a sequence-independent or sequence-dependent manner. Further, it has been reported that these various immunostimulatory RNAs stimulate TLR3, TLR7, or TLR8.

Viral-derived double-stranded RNA (dsRNA) and poly I:C, a synthetic analog of dsRNA, were recently reported to be ligands of TLR3. Alexopoulou L et al. (2001) Nature 413:732-8. Even more recently, Lipford and coworkers disclosed that certain G,U-containing RNA sequences are immunostimulatory, acting through stimulation of TLR7 and TLR8. Heil F et al. (2004) Science 303:1526-9, and U.S. Pat. Appl. 2003/0232074 A1.

Heil et al. reported that guanosine- and uridine-rich phosphorothioate ssRNA oligonucleotides, derived from HIV-1 and complexed with the cationic lipid DOTAP, stimulate dendritic cells (DC) and macrophages to secrete interferon alpha (IFN-α), tumor necrosis factor (TNF), interleukin 12 (IL-12), and interleukin 6 (IL-6). Heil F et al. (2004) Science 303:1526-9. Murine TLR7 was reported to confer responsiveness to GU-rich ssRNA, and human TLR8 was reported to confer responsiveness to GU-rich and U-rich ssRNA. Although specific sequences were tested, no motif was identified. Ibid.

Diebold et al. recently reported that single-stranded RNA (ssRNA) of viral or synthetic origin activates TLR7. Diebold S S et al. (2004) Science 303:1529-31. They reported that viral genomic ssRNA from influenza virus, as well as polyU, triggers IFN-αproduction by plasmacytoid dendritic cells (pDC). No sequence-specific motif was identified beyond polyU. Mouse spleen and some short ssRNA oligos (of the type used to make short interfering dsRNA) also induced IFN-α. Ibid.

Lund et al. recently reported that murine TLR7 recognizes two single-stranded RNA viruses: VSV and influenza virus. Lund J M et al. (2004) Proc Natl Acad Sci USA 101:5598-603. They reported that recognition requires intact endocytic pathways and myeloid differentiation factor 88 (MyD88), involves pDCs, and results in expression of type 1 IFN (e.g., IFN-α). The exact viral ligand that triggers TLR7 was not identified. Ibid.

Scheel et al. recently reported that stabilized messenger RNA and synthetic RNA activate mouse DC, but not B cells, and promote Th1 immune responses in vitro. Scheel B et al. (2004) Eur J Immunol 34:537-47. RNA stabilized with cationic protein (protamine) was reported to be significantly more stable than phosphorothioate-stabilized RNA. In addition, CpG RNA plus antigen was found to induce a Th2-type antibody response (IgG1) in vivo in BALB/c mice. The authors speculated a TLR other than TLR3 or TLR9 is involved, particularly TLR 7 or TLR8; however, no RNA sequence motif was identified. Ibid.

WO 2004/004743 (to CureVac) discloses compositions and methods involving immunostimulatory RNA that includes at least one chemical modification, e.g., modified internucleotide linkage (phosphorothioate) or nucleobase (including inosine, 5-methylcytosine, and 7-deazaguanosine). Claimed compositions include the nucleic acids alone, with adjuvant (including CpG oligodeoxynucleotide), and with antigen (including nucleic acid encoding the antigen). Claimed methods include production of composition or vaccine for prevention/treatment of infectious disease and cancer.

There has also been recent intense interest in sequence-dependent RNA as a therapeutic principle in additional contexts, beginning with the development of antisense and more recently including RNA interference (RNAi). These evolving technologies are directed in general to the control of gene expression, especially gene silencing.

Currently, clinical and experimental applications involving RNA are limited by the characteristic highly labile nature of RNA in vivo and in vitro. RNA is highly susceptible to degradation by nucleases. Nucleases generally include exonucleases and endonucleases, which degrade internucleotide phosphate linkages at the ends of nucleic acid molecules and at internal sites, respectively. The rate of degradation can vary depending on the location of the nucleic acid, e.g., outside the cell (rapid) versus inside the cell (generally slower), as well as in the latter case the intracellular compartment containing the nucleic acid, e.g., intraliposomal (rapid) versus intracytoplasmic (slower). The rate of nuclease-mediated degradation can also vary depending on the source of enzyme. For example, oligonucleotides are generally completely degraded in 15 minutes in fetal calf serum. In order to overcome such limitations of RNA, a number of approaches have been reported to generate stabilized forms of RNA and DNA. See, for example, Uhlmann E et al. (1990) Chem Rev 90:543-84. Unfortunately, many of these approaches have not resulted in satisfactory alternatives, either because the stability gained is insufficient or because the gain in stability is associated with loss of function.

### SUMMARY OF THE INVENTION

The present invention is based in part on the unexpected discovery by the inventors that phosphorothioate-modified oligoribonucleotides (ORN) are rapidly degraded within minutes in serum, and are thus more labile to nucleases than phosphorothioate-modified oligodeoxynucleotides (ODN) and also more labile to nucleases than unmodified (phosphodiester-linked) ODN. The present invention provides chemically modified oligoribonucleotides and ORN analogs characterized by their improved stability to nucleases and/or their improved biological activity compared to corresponding naturally occurring RNA molecules. As used herein, modified oligoribonucleotides and ORN analogs of the invention shall be referred to collectively as modified oligoribonucleotide analogs.

The invention relates generally to immunostimulatory modified oligoribonucleotide analogs that contain certain immunostimulatory RNA motifs, as well as to related immunostimulatory compositions containing such immunostimulatory modified oligoribonucleotide analogs, and methods for the use of such immunostimulatory modified oligoribonucleotide analogs and compositions. The modified oligoribonucleotide analogs of the invention are useful in any setting or application that calls for a composition or method for stimulating or augmenting an immune response. As disclosed below, the modified oligoribonucleotide analogs of the invention are of particular use in the preparation of pharmaceutical compositions, including adjuvants, vaccines, and other medicaments for use in treating a variety of conditions, including infection, cancer, allergy, and asthma. The invention in certain aspects thus relates to immunostimulatory compositions that include immunostimulatory modified oligoribonucleotide analogs of the invention, as well as methods of their use. Also as disclosed below, the modified oligoribonucleotide analogs of the invention are of particular use in methods for activating an immune cell, vaccinating a subject, treating a subject having an immune system deficiency, treating a subject having an infection, treating a subject having cancer, treating a subject having an allergic condition, treating a subject having asthma, airway remodeling, promoting epitope spreading, and antibody-dependent cellular cytotoxicity (ADCC).

The modified oligoribonucleotide analogs of the invention include at least one chemical modification that distinguishes them from naturally occurring RNA. The modification can involve a modified internucleotide phosphate linkage, a modified sugar, a modified nucleobase, a nucleotide analog, or any combination thereof.

As disclosed in greater detail below, the modified oligoribonucleotide analogs of the invention are also characterized by their inclusion of at least one sequence-dependent immunostimulatory motif. The sequence-dependent immunostimulatory motif generally is a short RNA sequence, although in certain embodiments the motif can also include a modification such as a modified internucleotide phosphate linkage, a modified nucleobase, a modified sugar, a nucleotide analog, or any combination thereof As described in detail below, in one embodiment the immunostimulatory motif occurs in the context of a longer modified oligoribonucleotide analog of the invention.

As will be evident from the foregoing, the modified oligoribonucleotide analogs of the invention are not naturally occurring compounds.

In one aspect the invention provides an immunostimulatory composition including a polymer 4 to 100 units long, wherein each unit includes a nucleoside or a nucleoside analog, wherein each pair of adjacent units is linked by a covalent linkage, and wherein the composition includes (a) an immunostimulatory RNA motif 4 to 8 nucleotides long, and (b) at least one modified phosphate linkage selected from the group consisting of:

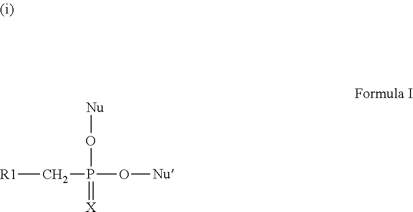

wherein
R1 is hydrogen (H), COOR, OH, C1-C18 alkyl, C₆H₅, or (CH₂)ₘ—NH—R2, wherein R is H or methyl, butyl, methoxyethyl, pivaloyl oxymethyl, pivaloyl oxybenzyl, or S-pivaloyl thioethyl; R2 is H, C1-C18 alkyl, or C2-C18 acyl; and m is to 17;
X is oxygen (O) or sulfur (S); and
each of Nu and Nu′ independently is a nucleoside or nucleoside analog;
with the proviso that if R1 is H, then X is S;

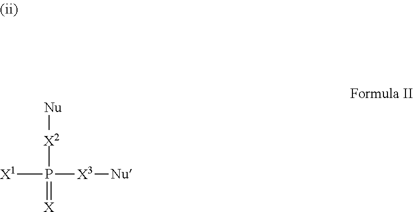

wherein
X is O or S;
X¹ is OH, SH, BH₃, OR3, or NHR3, wherein R3 is C1-C18 alkyl;
each of X² and X³ independently is O, S, CH₂, or CF₂; and
each of Nu and Nu′ independently is a nucleoside or nucleoside analog; with the proviso that
(a) at least one of X, X², and X³ is not O or X¹ is not OH,
(b) if X¹ is SH, then at least one of X, X², and X³ is not O,
(c) if X and X² are O and if X¹ is OH, then X³ is not S and Nu is 3′Nu and Nu′ is 5′Nu′, and
(d) if X′ is BH₃, then at least one of X, X², or X³ is S; and

(iii) any combination of (i) and (ii).

Specific embodiments of Formula I are encompassed by this aspect of the invention. In an embodiment according to this aspect of the invention in Formula I X is S and R1 is H. In an embodiment according to this aspect of the invention in Formula I X is O and R1 is COOH. In an embodiment according to this aspect of the invention in Formula I X is O and R1 is (CH₂)ₘ—NH—R2, wherein m is an integer from 1 to 17, inclusive, and wherein R2 is H, C1-C18 alkyl, or C2-C18 acyl. In an embodiment according to this aspect of the invention in Formula I X is O and R1 is C1-C18 alkyl. Also according to this aspect of the invention in one embodiment Formula I is

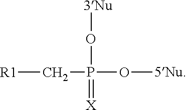

Specific embodiments of Formula II are also encompassed by this aspect of the invention. In one embodiment according to this aspect of the invention in Formula II X and X³ are O, X¹ is OH, and X² is S or CH₂.

In one embodiment according to this aspect of the invention in Formula II X is O, X¹ is SH, X² is O, and X³ is S.

In an embodiment according to this aspect of the invention at least one modified phosphate linkage is Formula I. In an embodiment according to this aspect of the invention at least one modified phosphate linkage is Formula II. Of course the immunostimulatory composition according to this aspect of the invention can include both at least one modified phosphate linkage provided as Formula I and at least one modified phosphate linkage provided as Formula II.

In an embodiment according to this aspect of the invention the RNA motif includes at least one of Nu and Nu′ in Formula I or in Formula II. In another embodiment according to this aspect of the invention the RNA motif excludes Nu and Nu′ in Formula I or in Formula II.

In one aspect the invention provides an immunostimulatory composition including a polymer 4 to 100 units long, wherein each unit includes a nucleoside or a nucleoside analog, wherein each pair of adjacent units is linked by a covalent linkage, and wherein the composition includes (a) an immunostimulatory RNA motif 4 to 8 nucleotides long, and (b) at least one nucleotide analog provided as Formula IIIA or Formula IIIB

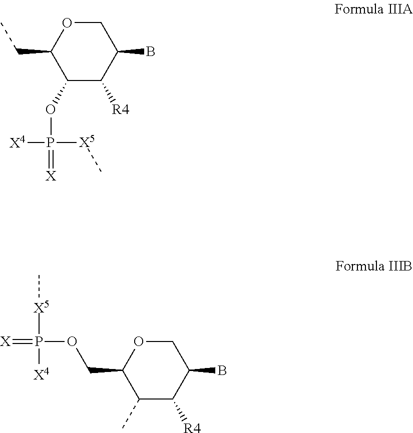

wherein
R4 is H or OR, wherein R is H or C1-C18 acyl;
B is a nucleobase, a modified nucleobase, or H;
each of X and X⁵ independently is O or S;
X⁴ is OH, SH, methyl, or NHR5, wherein R5 is C1-C18 alkyl; and
each dashed line independently represents an optional bond to an adjacent unit, hydrogen, or an organic radical;
   with the proviso that at least one of X and X⁵ is not O or X⁴ is not OH. An organic radical refers to a group selected, for example, from hydroxyl, acylated hydroxy, phosphate, and a lipophilic residue as disclosed herein.

In one embodiment according to this aspect of the invention in Formula IIIA or Formula IIIB R4 is OH, X⁴ is SH, and each of X and X⁵ is O.

In one aspect the invention provides an immunostimulatory composition including features of both the first and the second aspects just described. Accordingly in one aspect the invention provides the immunostimulatory composition of the first aspect described above, further including at least one nucleotide analog provided as Formula IIIA or Formula IIIB

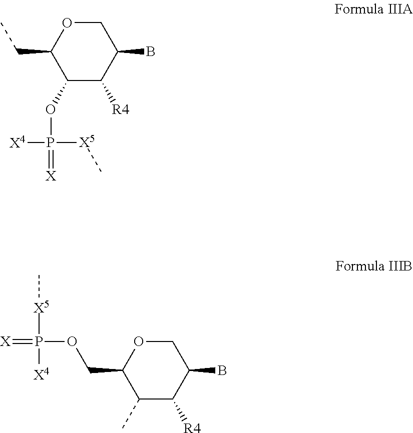

wherein
R4 is H or OR, wherein R is H or C1-C18 acyl;
B is a nucleobase, a modified nucleobase, or H;
each of X and X⁵ independently is O or S;
X⁴ is OH, SH, methyl, or NHR5, wherein R5 is C1-C18 alkyl; and
each dashed line independently represents an optional bond to an adjacent unit, hydrogen, or an organic radical;
   with the proviso that at least one of X and X⁵ is not O or X⁴ is not OH.

In one embodiment according to this aspect of the invention in Formula IIIA or Formula IIIB R4 is OH, X⁴ is SH, and each of X and X⁵ is O.

Also according to these and other aspects of the invention, in various embodiments the immunostimulatory RNA motif has a base sequence selected from
(i) 5′-C/U-U-G/U-U-3′,
(ii) 5′-R-U-R-G-Y-3′,
(iii) 5′-G-U-U-G-B-3′,
(iv) 5′-G-U-G-U-G/U-3′, and
(v) 5′-G/C-U-A/C-G-G-C-A-C-3′,
   wherein C/U is cytosine (C) or uracil (U), G/U is guanine (G) or U, R is purine, Y is pyrimidine, B is U, G, or C, G/C is G or C, and A/C is adenine (A) or C.

In various embodiments 5′-CIU-U-G/U-U-3′ is CUGU, CUUU, UUGU, or UUUU.

In various embodiments 5′-R-U-R-G-Y-3′ is GUAGU, GUAGC, GUGGU, GUGGC, AUAGU, AUAGC, AUGGU, or AUGGC. In one embodiment the base sequence is GUAGUGU.

In various embodiments 5′-G-U-U-G-B-3′ is GUUGU, GUUGG, or GUUGC.

In various embodiments 5′-G-U-G-U-G/U-3′ is GUGUG or GUGUU. In one embodiment the base sequence is GUGUUUAC.

In various embodiments 5′-G/C-U-A/C-G-G-C-A-C-3′ is GUAGGCAC, GUCGGCAC, CUAGGCAC, or CUCGGCAC.

In one embodiment the modified oligoribonucleotide analog has a base sequence provided as 5′-GUUGUGGUUGUGGUUGUG-3′ (SEQ ID NO:1).

In certain embodiments the modified oligoribonucleotide analog can exclude any one or more of the aforementioned immunostimulatory RNA motifs. For example, in one embodiment the modified oligoribonucleotide analog excludes an immunostimulatory RNA motif having a base sequence provided by 5′-C/U-U-G/U-U-3′.

In one aspect the invention provides an immunostimulatory composition including a modified oligoribonucleotide analog of the invention and an adjuvant. In various embodiments the adjuvant is an adjuvant that creates a depot effect, an immune-stimulating adjuvant, or an adjuvant that creates a depot effect and stimulates the immune system. In one embodiment the immunostimulatory composition according to this aspect of the invention is a conjugate of the modified oligoribonucleotide analog and the adjuvant. In one embodiment according to this aspect of the invention the modified oligoribonucleotide analog is covalently linked to the adjuvant.

The compositions of the invention can optionally include an antigen. Thus in one aspect the invention provides a vaccine, wherein the vaccine includes a modified oligoribonucleotide analog of the invention and an antigen. In one aspect the invention provides a vaccine that includes a conjugate of a modified oligoribonucleotide analog of the invention and an antigen. In one embodiment the conjugate according to this aspect of the invention includes the modified oligoribonucleotide analog covalently linked to the antigen. In various embodiments the antigen can be an antigen per se or it can include a nucleic acid encoding the antigen. The antigen can be any antigen, including a cancer antigen, a microbial antigen, or an allergen.

In one aspect the invention provides an immunostimulatory composition including a conjugate of a modified oligoribonucleotide analog of the invention and a lipophilic moiety. In one embodiment the modified oligoribonucleotide analog is covalently linked to the lipophilic moiety. In one embodiment the lipophilic moiety is selected from the group consisting of cholesteryl, palmityl, and fatty acyl. In one embodiment the lipophilic moiety is a derivative of cholesterol, e.g., cholesteryl.

In one aspect the invention provides an immunostimulatory composition that includes a chimeric DNA:“RNA” molecule (hereinafter, referred to simply as a chimeric DNA:RNA molecule) that includes a DNA and a modified ORN analog of the invention. In one embodiment a DNA component of the chimeric DNA:RNA molecule includes an immunostimulatory CpG nucleic acid, i.e., a TLR9 agonist. In one embodiment the DNA and “RNA” portions of the chimeric DNA:RNA molecule are covalently linked through an internucleotide phosphate bond. In another embodiment the DNA and “RNA” portions of the chimeric DNA:RNA molecule are covalently linked through a linker, e.g., a non-nucleotidic linker.

The DNA of a chimeric DNA:RNA molecule as used herein specifically includes DNA with a phosphodiester backbone, DNA with a phosphorothioate backbone, DNA incorporating at least one additional modification of phosphate linkage, sugar, and/or nucleobase as disclosed herein, as well as any combination of the foregoing. In one embodiment the DNA of a chimeric DNA:RNA molecule as used herein specifically includes a phosphorothioate backbone.

In one aspect the invention provides an immunostimulatory composition that includes a covalently closed, partially single-stranded, dumbbell-shaped nucleic acid molecule, wherein at least one single-stranded portion of the molecule includes an immunostimulatory RNA motif of the invention. In one embodiment the nucleic acid molecule is a chimeric DNA:RNA molecule. In one embodiment the nucleic acid molecule is a chimeric DNA:RNA molecule wherein at least one single-stranded portion of the molecule includes an immunostimulatory RNA motif of the invention and wherein at least one single-stranded portion of the molecule includes an immunostimulatory CpG DNA motif.

In one aspect the invention provides a pharmaceutical composition including a composition of any of the foregoing aspects of the invention and a pharmaceutically acceptable carrier.

In one aspect the invention provides a pharmaceutical composition including a composition of any of the foregoing aspects of the invention, in association with a delivery vehicle chosen from a cationic lipid, a liposome, a cochleate, a virosome, an immune-stimulating complex (ISCOM), a microparticle, a microsphere, a nanosphere, a unilamellar vesicle (LUV), a multilamellar vesicle, an oil-in-water emulsion, a water-in-oil emulsion, an emulsome, and a polycationic peptide, and, optionally, a pharmaceutically acceptable carrier. In one embodiment according to this aspect of the invention the pharmaceutical composition includes an antigen.

Further according to these and other aspects of the invention, in various embodiments the composition can optionally include at least one 5′-5′ internucleotide linkage, at least one 3′-3′ internucleotide linkage, at least one 5′-5′ internucleotide linkage that includes a linker moiety, at least one 3′-3′ internucleotide linkage that includes a linker moiety, or any combination thereof.

Further still according to these and other aspects of the invention, in various embodiments the composition can optionally include at least one 2′-2′ internucleotide linkage, at least one 2′-3′ internucleotide linkage, at least 2′-5′ internucleotide linkage, or any combination thereof. In a preferred embodiment the at least one 2′-2′ internucleotide linkage, at least one 2′-3′ internucleotide linkage, or at least 2′-5′ internucleotide linkage occurs outside of the immunostimulatory RNA motif.

Also according to these and other aspects of the invention, the modified oligoribonucleotide analog in one embodiment includes at least one multiplier unit. Accordingly, in certain embodiments the modified oligoribonucleotide analog of the invention can have a branched structure. Branched compositions can include 3′-5′, 5′-5′, 3′-3′, 2′-2′, 2′-3′, or 2′-5′ internucleotide linkages, in any combination. In one embodiment the modified oligoribonucleotide analog includes at least two multiplier units, resulting in a so-called dendrimer. In addition, in certain embodiments the modified oligoribonucleotide analog of the invention may include two or more immunostimulatory RNA motifs, arranged for example in tandem along a linear modified oligoribonucleotide analog, on different arms of a branched structure, or both in tandem along a linear modified oligoribonucleotide analog and on different arms of a branched structure. Branched structures, including dendrimers, can optionally include at least one immunostimulatory CpG nucleic acid, for example as a separate arm of a branched structure.

Also according to these and other aspects of the invention, in one embodiment the composition includes a modified nucleobase outside of the immunostimulatory RNA motif, wherein the modified nucleobase is selected from the group consisting of hypoxanthine, inosine, 8-oxo-adenine, 7-substituted derivatives thereof, dihydrouracil, pseudouracil, 2-thiouracil, 4-thiouracil, 5-aminouracil, 5-(C₁-C₆)-alkyluracil, 5-methyluracil, 5-(C₂-C₆)-alkenyluracil, 5-(C₂-C₆)-alkynyluracil, 5-(hydroxymethyl)uracil, 5-chlorouracil, 5-fluorouracil, 5-bromouracil, 5-hydroxycytosine, 5-(C₁-C₆)-alkylcytosine, 5-methylcytosine, 5-(C₂-C₆)-alkenylcytosine, 5-(C₂-C₆)-alkynylcytosine, 5-chlorocytosine, 5-fluorocytosine, 5-bromocytosine, N²-dimethylguanine, 7-deazaguanine, 8-azaguanine, 7-deaza-7-substituted guanine, 7-deaza-7-(C2-C6)alkynylguanine, 7-deaza-8-substituted guanine, 8-hydroxyguanine, 6-thioguanine, 8-oxoguanine, 2-aminopurine, 2-amino-6-chloropurine, 2,4-diaminopurine, 2,6-diaminopurine, 8-azapurine, substituted 7-deazapurine, 7-deaza-7-substituted purine, 7-deaza-8-substituted purine, hydrogen (abasic residue), and any combination thereof.

Further according to these and other aspects of the invention, in one embodiment the composition includes a modified U nucleobase selected from the group consisting of dihydrouracil, pseudouracil, 2-thiouracil, 4-thiouracil, 5-aminouracil, 5-(C₁-C₆)-alkyluracil, 5-methyluracil, 5-(C₂-C₆)-alkenyluracil, 5-(C₂-C₆)-alkynyluracil, 5-(hydroxymethyl)uracil, 5-chlorouracil, 5-fluorouracil, 5-bromouracil, and any combination thereof. In various embodiments the modified U nucleobase can be inside the immunostimulatory RNA motif, outside of the immunostimulatory RNA motif, or both inside and outside of the immunostimulatory RNA motif.

Also according to these and other aspects of the invention, in one embodiment the composition includes a modified G nucleobase selected from the group consisting of N²-dimethylguanine, 7-deazaguanine, 8-azaguanine, 7-deaza-7-substituted guanine, 7-deaza-7-(C2-C6)alkynylguanine, 7-deaza-8-substituted guanine, 8-hydroxyguanine, 6-thioguanine, 8-oxoguanine, and any combination thereof. In one embodiment the modified G nucleobase is 8-hydroxyguanine. In various embodiments the modified G nucleobase can be inside the immunostimulatory RNA motif, outside of the immunostimulatory RNA motif, or both inside and outside of the immunostimulatory RNA motif.

In certain embodiments at least one β-ribose unit may be replaced by β-D-deoxyribose or a modified sugar unit, wherein the modified sugar unit is for example selected from β-D-ribose, α-D-ribose, β-L-ribose (as in ‘Spiegelmers’), α-L-ribose, 2′-amino-2′-deoxyribose, 2′-fluoro-2′-deoxyribose, 2′-O-(C1-C6)alkyl-ribose, preferably 2′-O-(C1-C6)alkyl-ribose is 2′-O-methylribose, 2′-O-(C2-C6)alkenyl-ribose, 2′-[O-(C1-C6)alkyl-O-(C1-C6)alkyl]-ribose, LNA and α-LNA (Nielsen P et al. (2002) Chemistry-A European Journal 8:712-22), β-D-xylo-furanose, α-arabinofuranose, 2′-fluoro arabinofuranose, and carbocyclic and/or open-chain sugar analogs (described, for example, in Vandendriessche et al. (1993) Tetrahedron 49:7223) and/or bicyclosugar analogs (described, for example, in Tarkov M et al. (1993) Helv Chim Acta 76:481). In various embodiments the β-D-deoxyribose or modified sugar unit can be inside the immunostimulatory RNA motif, outside of the immunostimulatory RNA motif, or both inside and outside of the immunostimulatory RNA motif. In one embodiment the β-D-deoxyribose or modified sugar unit is outside of the immunostimulatory RNA motif.

Modified oligoribonucleotide analogs in which at least one ribose unit is replaced by 1,5-anhydrohexitol (Bouvere B et al. (1997) Nucleosides Nucleotides 16:973-6) or by D-Altritol (Allart B et al. (1999) Chemistry-A European Journal 5:2424-31) are also embodiments of this invention. In another embodiment, the modified oligoribonucleotide analog comprises at least one β-D-ribopyranosyl unit (“pyranosyl-RNA”; Pitsch S et al. (2003) Helv Chim Acta 86:4270-363). Alternatively, other ring-expanded or ring-condensed sugar analogs may replace ribose.

In another embodiment, at least one hydroxy group, preferably the 2′-hydroxy group, of the ribose unit is protected as a pro-drug, which is cleaved in vivo to release the oligomer with unprotected ribose. Known pro-drugs of ribose are e.g. the corresponding valinates (Kong L et al. (2003) Antivir Chem Chemother 14:263-70), formates (Repta A et al. (1975) J Pharm Sci 64:392-6), or isopropyl ethers (Winkelmann E et al. (1988) Arzneimittelforschung 38:1545-8).

Further according to these and other aspects of the invention, in one embodiment the polymer does not include a CG DNA dinucleotide, e.g., a CpG DNA dinucleotide.

Further according to these and other aspects of the invention, in one embodiment the composition further includes a polyG sequence covalently linked to at least one end of the immunostimulatory modified oligoribonucleotide analog, wherein each polyG sequence independently includes 3-12 consecutive guanosine nucleosides selected from the group consisting of guanosine ribonucleoside, guanosine deoxyribonucleoside, and any combination thereof. In one embodiment the polyG sequence is (dG)ₙ, wherein each dG is deoxyguanosine and n is an integer between 3 and 12, inclusive. In one embodiment the polyG sequence is (dG)ₙ, wherein n is an integer between 3 and 6, inclusive. In one embodiment the polyG sequence is (dG)ₙ, wherein the (dG)ₙ is a 3′ end of the immunostimulatory modified oligoribonucleotide analog. In one embodiment the polyG sequence is (dG)ₙ, wherein the (dG)ₙ is a 5′ end of the immunostimulatory modified oligoribonucleotide analog. In one embodiment a polyG sequence (dG)ₙ is a 3′ end of the immunostimulatory modified oligoribonucleotide analog and a polyG sequence (dG)ₙis a 5′ end of the immunostimulatory modified oligoribonucleotide analog.

Also according to these and other aspects of the invention, in one embodiment the polymer includes a sequence of nucleosides, nucleoside analogs, or a combination of nucleosides and nucleoside analogs capable of forming secondary structure provided by at least two adjacent hydrogen-bonded base pairs. In one embodiment the secondary structure is a stem-loop secondary structure.

In one aspect the invention provides a method of activating an immune cell. The method according to this aspect of the invention includes the step of contacting an immune cell with an effective amount of a composition of the invention.

In one aspect the invention provides a method of treating a subject having an immune system deficiency. The method according to this aspect of the invention includes the step of administering to the subject an effective amount of a composition of the invention. In one embodiment the composition is an modified oligoribonucleotide analog of the invention.

In one aspect the invention provides a method of vaccinating a subject. The method according to this aspect of the invention includes the step of administering to the subject an antigen and a modified oligoribonucleotide analog of the invention. In one embodiment the administering the antigen includes administering a nucleic acid encoding the antigen.

In one embodiment the antigen and modified oligoribonucleotide analog are administered as individual compositions. In another embodiment the antigen and modified oligoribonucleotide analog are administered as a single composition.

In another aspect the invention provides a method for inducing a Thl-like immune response in a subject. The method according to this aspect of the invention includes the step of administering to the subject an effective amount of a composition of the invention. In one embodiment the method includes the step of administering to the subject an effective amount of a modified oligoribonucleotide analog of the invention. In one embodiment the method includes the step of administering to the subject an effective amount of a modified oligoribonucleotide analog of the invention and an antigen. In one embodiment according to this aspect of the invention a Th1-like immune response is a Th1 immune response.

In one aspect the invention provides a method for suppressing a Th2-like immune response in a subject. The method according to this aspect of the invention includes the step of administering to the subject an effective amount of a composition of the invention. In one embodiment the method includes the step of administering to the subject an effective amount of a modified oligoribonucleotide analog of the invention. In one embodiment the method includes the step of administering to the subject an effective amount of a modified oligoribonucleotide analog of the invention and an antigen. In one embodiment according to this aspect of the invention a Th2-like immune response is a Th2 immune response.

In one aspect the invention provides a method for treating a subject having or at risk of having an infectious disease. The method according to this aspect of the invention includes the step of administering to the subject an effective amount of a composition of the invention. In one embodiment the method includes the step of administering to the subject an effective amount of a modified oligoribonucleotide analog of the invention. In one embodiment the method includes the step of administering to the subject an effective amount of a modified oligoribonucleotide analog of the invention and a microbial antigen.

In one aspect the invention provides a method for treating a subject having or at risk of having a cancer. The method according to this aspect of the invention includes the step of administering to the subject an effective amount of a composition of the invention. In one embodiment the method includes the step of administering to the subject an effective amount of a modified oligoribonucleotide analog of the invention. In one embodiment the method includes the step of administering to the subject an effective amount of a modified oligoribonucleotide analog of the invention and a cancer antigen.

In one aspect the invention provides a method for treating a subject having or at risk of having an allergic condition. The method according to this aspect of the invention includes the step of administering to the subject an effective amount of a composition of the invention. In one embodiment the method includes the step of administering to the subject an effective amount of a modified oligoribonucleotide analog of the invention. In one embodiment the method includes the step of administering to the subject an effective amount of a modified oligoribonucleotide analog of the invention and an allergen.

In one aspect the invention provides a method for treating a subject having or at risk of having asthma. The method according to this aspect of the invention includes the step of administering to the subject an effective amount of a composition of the invention. In one embodiment the method includes the step of administering to the subject an effective amount of a modified oligoribonucleotide analog of the invention. In one embodiment the method includes the step of administering to the subject an effective amount of a modified oligoribonucleotide analog of the invention and an allergen.

In another aspect the invention provides a method for treating a subject having airway remodeling. The method according to this aspect of the invention includes the step of administering to the subject an effective amount of a modified oligoribonucleotide analog of the invention.

In one aspect the invention provides a method for increasing antibody-dependent cellular cytotoxicity (ADCC). The method according to this aspect of the invention includes the step of administering to a subject in need of increased ADCC an effective amount of a modified oligoribonucleotide analog of the invention and an antibody to increase ADCC. In one embodiment the antibody is an antibody specific for a cancer antigen or other antigen expressed by a cancer cell. In one embodiment the antibody is an IgG antibody.

The invention in one aspect provides a method for enhancing epitope spreading. The method according to this aspect of the invention includes the sequential steps of contacting a cell of the immune system with an antigen and subsequently contacting the cell with at least two doses of a modified oligoribonucleotide analog of the invention. In one embodiment the method is performed in vivo. The method in one embodiment includes the steps of administering to a subject a vaccine that includes an antigen and an adjuvant and subsequently administering to the subject at least two doses of a modified oligoribonucleotide analog of the invention, in an effective amount to induce multiple epitope-specific immune responses. The method in one embodiment includes the steps of administering to a subject a vaccine that includes a tumor antigen and an adjuvant and subsequently administering to the subject at least two doses of a modified oligoribonucleotide analog of the invention, in an effective amount to induce multiple epitope-specific immune responses. The method in one embodiment involves applying a therapeutic protocol which results in immune system antigen exposure in a subject, followed by administering at least two doses of a modified oligoribonucleotide analog of the invention, in an effective amount to induce multiple epitope-specific immune responses. In various embodiments the therapeutic protocol is surgery, radiation, chemotherapy, other cancer medicaments, a vaccine, or a cancer vaccine. In one embodiment the at least two doses of the modified oligoribonucleotide analog are administered at least one day to one week apart from one another. In one embodiment the at least two doses of the modified oligoribonucleotide analog are administered at least one week to one month apart from one another. In one embodiment the at least two doses of the modified oligoribonucleotide analog are administered at least one month to six months apart from one another.

In one aspect the invention provides a method for identifying a candidate inhibitor of TLR signaling. The method according to this aspect of the invention includes the steps of contacting a TLR chosen from TLR7 and TLR8 with a modified oligoribonucleotide analog of the invention in presence of a test agent and measuring a test signal mediated by the TLR, comparing the test signal to a control signal mediated by the TLR after contacting the TLR with the modified oligoribonucleotide analog in absence of the test agent, and identifying the test agent as a candidate inhibitor of signaling by the TLR when the test signal is less than the control signal. In one embodiment the TLR is expressed by a cell. In one embodiment the TLR is TLR7. In one embodiment the TLR is TLR8.

These and other features of the invention will be described in further detail in connection with the detailed description of the invention.

### BRIEF DESCRIPTION OF THE DRAWINGS

FIG. 1 is a graph depicting interferon alpha (IFN-a) produced by human peripheral blood mononuclear cells (PBMC) following incubation for 24 hours in the presence of the indicated concentrations of various oligonucleotides, lipopolysaccharide (LPS), or no additive (w/o), in absence of the cationic lipid DOTAP. Sequences of the various oligonucleotides are described in Example 1. D2241, D2242, and D2243 refer to individual donors of PBMC.

FIG. 2 is a graph depicting IFN-α produced by human PBMC following incubation for 24 hours in the presence of the indicated concentrations of various oligonucleotides and 20 μg/ml DOTAP, or in the presence of the indicated concentrations of DOTAP alone. Sequences of the various oligonucleotides are described in Example 1. D2241, D2242, and D2243 refer to individual donors of PBMC.

FIG. 3 is a graph depicting tumor necrosis factor alpha (TNF-α) produced by human PBMC following incubation for 24 hours in the presence of the indicated amounts of various oligonucleotides, lipopolysaccharide (LPS), or no additive (w/o), in absence of DOTAP. Sequences of the various oligonucleotides are described in Example 1. D2241, D2242, and D2243 refer to individual donors of PBMC.

FIG. 4 is a graph depicting TNF-α produced by human PBMC following incubation for 24 hours in the presence of the indicated concentrations of various oligonucleotides and 20 μg/ml DOTAP, or in the presence of the indicated concentrations of DOTAP alone. Sequences of the various oligonucleotides are described in Example 1. D2241, D2242, and D2243 refer to individual donors of PBMC.

FIG. 5 is a structural formula for 5′-DMT-2′-O-Cpep-5′-thio-uridine-3′-phosphoramidite, wherein Cpep is 1-(4-chlorophenyl)-4-ethoxypiperidin-4-yl and DMT is dimethoxytrityl.

### DETAILED DESCRIPTION OF THE INVENTION

The invention relates in part to the discovery by the inventors of a number of RNA-like molecules that are effective as.immunostimulatory compounds. Identification of the immunostimulatory compounds arose through a systematic effort aimed at improving the immunostimulatory capacity of certain specific motif-containing RNAs. As a result of this effort, it has now been discovered that RNA-like molecules containing an immunostimulatory motif and certain modifications involving certain modified phosphate linkages, certain nucleotide analogs, or both modified phosphate linkages and nucleotide analogs, are important immunostimulatory compounds. It has now been discovered that molecules containing an immunostimulatory RNA motif are, alone or in combination with certain other components, important immunostimulatory compounds that find use in a number of methods for treating subjects having or at risk of having a condition in which it would be advantageous to induce, augment, or redirect an immune response. As used herein, an immunostimulatory composition of the invention includes a modified oligoribonucleotide (ORN) analog of the invention. In one embodiment an immunostimulatory composition of the invention is a modified oligoribonucleotide (ORN) analog of the invention.

It was previously discovered that certain sequence-dependent RNA motifs are immunostimulatory, acting through TLR7, TLR8, and/or TLR3. These immunostimulatory RNA motifs include 5′-C/U-U-G/U-U-3′, 5′-R-U-R-G-Y-3′, 5′-G-U-U-G-B-3′, 5′-G-U-G-U-G/U-3′, and 5′-G/C-U-A/C-G-G-C-A-C-3′, wherein C/U is cytosine (C) or uracil (U), G/U is guanine (G) or U, R is purine, Y is pyrimidine, B is U, G, or C, GIC is G or C, and A/C is adenine (A) or C. Importantly, in addition to being sequence-specific, the immunostimulatory RNA motifs are effective as single-stranded RNA, partially double-stranded RNA, or wholly double-stranded RNA, and their immunostimulatory effect can be abrogated by RNAse treatment. It was previously discovered that certain single-stranded G,U-rich RNAs as short as just 5 nucleotides long can stimulate immune cells to produce large amounts of a number of cytokines and chemokines, including tumor necrosis factor alpha (TNF-α), interleukin 6 (IL-6), interleukin 12 (IL-12), type 1 interferon (e.g., interferon alpha (IFN-α)), interferon gamma (IFN-γ), and IFN-γ-inducible protein 10 (IP-10).

Although the modified oligoribonucleotide analogs of the invention can include natural RNA sequences, e.g., the immunostimulatory RNA motif, the analogs generally are not RNA but rather are polymers made up of nucleoside or nucleoside analogs linked together by covalent linkages. In one embodiment the modified oligoribonucleotide analog is a linear polymer with free ends. In another embodiment the modified oligoribonucleotide analog is a linear polymer that is circular, i.e., without free ends. In yet another embodiment the modified oligoribonucleotide analog is a branched polymer.

As used herein, the terms “RNA” and equivalently “natural RNA” shall refer to two or more ribonucleotides (i.e., molecules each comprising a ribose sugar linked to a phosphate group and to a purine or pyrimidine nucleobase (e.g., guanine, adenine, cytosine, or uracil)) covalently linked together by 3′-5′ phosphodiester linkage(s).

As used herein, “nucleoside” refers to a single sugar moiety (e.g., ribose or deoxyribose) linked to an exchangeable organic base, with is either a substituted pyrimidine (e.g., cytosine (C), thymine (T) or uracil (U)) or a substituted purine (e.g., adenine (A) or guanine (G)).

As used herein, “nucleoside analog” refers to a single sugar moiety or analog thereof (e.g., ribose, deoxyribose, modified ribose, modified deoxyribose, six-membered sugar analog, or open-chain sugar analog) linked to an exchangeable organic base or analog thereof (e.g., either a substituted pyrimidine (e.g., cytosine (C), thymine (T) or uracil (U)), a substituted purine (e.g., adenine (A) or guanine (G)), a modified pyrimidine, a modified purine, or a hydrogen atom), wherein at least the sugar moiety or at least the exchangeable organic base is an analog or modified entity, compared to the corresponding sugar or base of an unmodified nucleoside selected from adenosine, guanosine, cytidine, thymidine, and uridine.

Individual units and ribonucleoside analogs of the modified oligoribouncleotide analogs of the invention may also be linked by non-nucleotidic linkers, in particular abasic linkers (dSpacers), triethylene glycol units, or hexaethylene glycol units. Additional linkers are alkylamino linkers, such as C3, C6, C12 amino linkers, and also alkylthiol linkers, such as C3 or C6 thiol linkers. The units and ribonucleoside analogs can also be linked by aromatic residues which may be further substituted by alkyl or substituted alkyl groups.

In various embodiments the immunostimulatory RNA motif can be 4, 5, 6, 7, or 8 nucleotides long. The immunostimulatory RNA motif in various embodiments can be, without limitation, 5′-C/U-U-G/U-U-3′, 5′-R-U-R-G-Y-3′, 5′-G-U-U-G-B-3′, 5′-G-U-G-U-G/U-3′, or 5′-G/C-U-A/C-G-G-C-A-C-3′, wherein C/U is cytosine (C) or uracil (U), G/U is guanine (G) or U, R is purine, Y is pyrimidine, B is U, G, or C, G/C is G or C, and A/C is adenine (A) or C.

The immunostimulatory RNA motif can occur at an end of the polymer (when the polymer has free ends). For example, a polymer with free ends and the immunostimulatory RNA motif positioned at an end of the polymer can be represented as XₐM or as MX_{b}, where M represents the immunostimulatory RNA motif and each of Xₐ and X_{b} independently represents one or more identical or nonidentical units of the polymer exclusive of the immunostimulatory RNA motif.

Alternatively, the immunostimulatory RNA motif can be flanked on both of its ends by at least one additional unit of the polymer, whether the polymer has free ends or not. For example, a polymer with free ends and units flanking the immunostimulatory RNA motif can be represented as XₐMX_{b}, where M represents the immunostimulatory RNA motif and each of Xₐ and X_{b} independently represents one or more identical or nonidentical units of the polymer exclusive of the immunostimulatory RNA motif.

In different embodiments the polymer including the immunostimulatory RNA motif can include a single motif or more than one immunostimulatory RNA motif. It is believed that there may be an advantage to having two or more immunostimulatory RNA motifs in a single polymer, for example if the motifs are spaced such that the polymer can engage two or more TLRs. For example, the polymer could engage two or more TLR7 receptors, or two or more TLR8 receptors, or at least one TLR7 receptor and at least one TLR8 receptor, thereby amplifying or modifying the resulting immunostimulatory effect.

When the polymer includes more than one immunostimulatory RNA motif, the polymer can be represented in one embodiment as M₁XM₂, wherein M₁ and M₂ each independently represent an immunostimulatory RNA motif and X represents one or more identical or nonidentical units of the polymer exclusive of the immunostimulatory RNA motifs. In one embodiment X includes a non-nucleotidic linker as described herein. In one embodiment X includes a branching unit as described herein.

When there is more than one immunostimulatory RNA motif in the polymer, the motifs generally can occur at any position along the polymer. For example, when there are two motifs, they may each occur at an end of the polymer. Alternatively, one motif can occur at an end and one motif can be flanked on both of its ends by at least one additional unit of the polymer. In yet another embodiment each motif can be flanked on both of its ends by at least one additional unit of the polymer.

Immunostimulatory modified oligoribonucleotide analogs of the invention can have sequences that include, but are not limited to the following, shown 5′ to 3′ reading left to right:

| | |
|---|---|
| UUUGUGUGUCUCUCUUGUUUUUGUGUGUCU | SEQ ID NO: 1 |
| UUUCCAAACAAGUCUCUUCUCUUGUUUGGU | SEQ ID NO: 2 |
| UUUAUCUAUCCUUAGCCAACUUUGUCUGGU | SEQ ID NO: 3 |
| UUUAUCUAUCCAUAGCCAACUUUUUCUGGU | SEQ ID NO: 4 |
| UUUAUCUAUCCUUAGCCAACUUUGUCUGGU | SEQ ID NO: 5 |
| UUGUCAUAUAAUUGGUUUUUUUGUCUUCGU | SEQ ID NO: 6 |
| UUGUAUUCAUUUUAAACUCCUGCUUUUGCU | SEQ ID NO: 7 |
| UUGUAUUCAUUUUAAACCCCUGCUUUUGCU | SEQ ID NO: 8 |
| UUGUAUUAGGAAUGGUUUUUUUGUCUUCGU | SEQ ID NO: 9 |
| UUGGAUUCAUUUUAAUCUCCUGCUUUUGCU | SEQ ID NO: 10 |
| UUGAUCUAUCCUUACCCAACUUUGUUUGGU | SEQ ID NO: 11 |
| UUGAACUAUCCUUACCCAACUUUGUUUGGU | SEQ ID NO: 12 |
| UUCCCAGACAAGUUUCUUCUCUUGUUUGGU | SEQ ID NO: 13 |
| UUCCCAAGCAAGUCUCUUCUCUUGUUUGGU | SEQ ID NO: 14 |
| UUCCAUUUUGGAUCAGUACCUGCUUUUGCU | SEQ ID NO: 15 |
| UUCCAUUUUGGAUCAGUACCUGCUUUCGCU | SEQ ID NO: 16 |
| UUCCAUUUUGAAUCAGUACCUGCUUUCGCU | SEQ ID NO: 17 |
| UUCCAUUUUGAAUCAGUACCUGCUUUCGCU | SEQ ID NO: 18 |
| UUCCAUUUCGGAUCAGUACCUGCUUUUGCU | SEQ ID NO: 19 |
| UUCCAUUUCGAAUCAGUACCUGCUUUCGCU | SEQ ID NO: 20 |
| UUCCAUUCUGAAUCAGUACCUGCUUUUGCU | SEQ ID NO: 21 |
| UUAUGGCAAAUCAAACGUAUCGCUUCUGCU | SEQ ID NO: 22 |
| UUAUGGCAAAUCAAACGCACCGCUUCUGCU | SEQ ID NO: 23 |
| UUAUCGUACCUUACAGAUUCUCUGUUUGGU | SEQ ID NO: 24 |
| UUAUCGUACCUCACAGAUUCUCUGUUUGGU | SEQ ID NO: 25 |
| UUAUCGUAACUUACGGAUUCUCUGUUUGGU | SEQ ID NO: 26 |
| UUAUCGUAACUCACGGAUUCUCUGUUUGGU | SEQ ID NO: 27 |
| UUAUCGUAACUCACCGAUUCUCUGUUUGGU | SEQ ID NO: 28 |
| UUAUAUUCAUCUUAAAGCUCCGCUUCUGCU | SEQ ID NO: 29 |
| UUACCAAGCAAGUUUCUUCUCUUGUUUGGU | SEQ ID NO: 30 |
| UGUUUUUUCUUUGAUCUGGUUGUUAAGCGU | SEQ ID NO: 31 |
| UGUGUCUUCUUUGAUCUGGUUGUUAAGCGU | SEQ ID NO: 32 |
| UGUAACAUAACUCAUCAUCUUUUAUGAUAC | SEQ ID NO: 33 |
| UGGUUGUUUUUAUUUUCCCCUGCUUUUGCU | SEQ ID NO: 34 |
| UGGUUGUAUUUAUUUUCCCCUGCUUUUGCU | SEQ ID NO: 35 |
| UGGUUGGUUUUAUUUUCCCCUGCUUUUGCU | SEQ ID NO: 36 |
| UGGUUGCUUUUAUUUUCCCCUGCUUUUGCU | SEQ ID NO: 37 |
| UGGUUGAUUUUAUUUUCCCCUGCUUUUGCU | SEQ ID NO: 38 |
| UGGUUGAUUUGAUUUCCCCCUGCUUUUGCU | SEQ ID NO: 39 |
| UGGUUGAUUUAAUUUUCCCCUGCUUUUGCU | SEQ ID NO: 40 |
| UGCUUCUUCUUUGGUUUUGUUGUUAAGCGU | SEQ ID NO: 41 |
| UGCAAGUUUGUUGUACGCAUUUUUUCCCGU | SEQ ID NO: 42 |
| UGCAAGUUUGUAGUACGCAUUUUUUCGCGU | SEQ ID NO: 43 |
| UGCAAGUUUGUAGUACGCAUUUUUUCGCGU | SEQ ID NO: 44 |
| UGAUUUUUAUAUGGUUUUUUUGUUAAGCGU | SEQ ID NO: 45 |
| UCUUCCAAGUAUCAUCAUCUUUUUUGAUAC | SEQ ID NO: 46 |
| UAUCCAUCUUGAAAAUAGCCAAUCUUAGCU | SEQ ID NO: 47 |
| UAUAUUCAUCUUAAAGGCUCCGCUUCUGCU | SEQ ID NO: 48 |
| UAUACCUAUCCUUACCCAGCUUUGUUUGGU | SEQ ID NO: 49 |
| UAGACCGAUCCUUACCCAACUUUGUUUGGU | SEQ ID NO: 50 |
| UAGAACGAUCCUUACCCAGCUUUGUCUGGU | SEQ ID NO: 51 |
| UAAUUGUAAUAAUGGUUUUUUUGUCUUCGU | SEQ ID NO: 52 |
| UAAUUGUAAGAAUGGUUUUUUUGUCUUCGU | SEQ ID NO: 53 |
| UAAUUAUAUUAAUGGUUUGUUUGUCUUCGU | SEQ ID NO: 54 |
| UAAUGUUAUCAAUGGUUUAUUUGUCUUCGU | SEQ ID NO: 55 |
| UAAUGGUAAUAAUGGUUUGUUUGUCUUCGU | SEQ ID NO: 56 |
| UAAUGAUAAUAAUGGUUUGUUUGUCUUCGU | SEQ ID NO: 57 |
| UAAGAAUGCUAUUGGUUUGUUUUUCUUCGU | SEQ ID NO: 58 |
| UAACUUAAUUUAUACGCGUUUUUUUCGCGU | SEQ ID NO: 59 |
| UAAAAAUUCUUCUUUCUUUUUGUGUGUCCG | SEQ ID NO: 60 |
| UAAAAAACCUUUUUUCUUUUUGUGUGUCCG | SEQ ID NO: 61 |
| GUUGCUUUUAUUUUCCCCUGCUUUUGCUAA | SEQ ID NO: 62 |
| GUGGAUAUUAGAAAAUGCUCUGCUUCUGCU | SEQ ID NO: 63 |
| GGUUGCUUUUAUUUUCCCCUGCUUUUGCUA | SEQ ID NO: 64 |
| GGAUUCAUUUUGAACUCCUGCUUUUGCUAA | SEQ ID NO: 65 |
| GGAUACAUAUCUCUUAAACUCUUGUCUGGU | SEQ ID NO: 66 |
| CUUUUCUUCUCUGGUUUUGUUGUUAAGCGU | SEQ ID NO: 67 |
| CUGAGCUUAGUCAAGUUACUUUUUUUAUAC | SEQ ID NO: 68 |
| CUGAGCUUAGUCAAGUUACUUUUCUUAUAC | SEQ ID NO: 69 |
| CUCAUCUUUCAAUAUCUACCUGCUUUUGCU | SEQ ID NO: 70 |
| CUCAUCUUUCAAUAUCUACCUGCUUUCGCU | SEQ ID NO: 71 |
| CUCAUCUUUCAACAUCUACCUGCUUUUGCU | SEQ ID NO: 72 |
| CUAAAAAUUCUUCUUUCUUUUUGUGUGCCC | SEQ ID NO: 73 |
| CGGUGAGUGAUUAUCUACCCUGCUUUUGCU | SEQ ID NO: 74 |
| CGGUGAGAGAUUAUCUACCCUGCUUUUGCU | SEQ ID NO: 75 |
| CGGUGAGAGAUUAUCUACCCUGCUUUUGCU | SEQ ID NO: 76 |
| CGCAAGUUUGUUGUACGCAUUUUUUCGCGU | SEQ ID NO: 77 |
| CCGAUAUCCCAUCUUCUUUUUCCCCUUGGU | SEQ ID NO: 78 |
| CCAUUAUGUCUUUGUCACCCUGCUUUUGCU | SEQ ID NO: 79 |
| CCAAUAUCCCAUCUUCAUUUUCCCCUUGGU | SEQ ID NO: 80 |
| CCAAUAUCCCAUAUUCAUUCUCCCCUUGGU | SEQ ID NO: 81 |
| CCAACAUCCCAUCUUCUUUUUCCCCUUGGU | SEQ ID NO: 82 |
| CAUUGAGUGAUUAUCUACCCUGCUUUUGCU | SEQ ID NO: 83 |
| CAUAUUGAAUAUAAUUGCGCUGCUUUCGCU | SEQ ID NO: 84 |
| CAUAUUGAAUAUAAUUGACCUGCUUUCGCU | SEQ ID NO: 85 |
| CAUAUUCAAUAUAAUUGACCUGCUUUUCGU | SEQ ID NO: 86 |
| CAGUGAGUGAUUAUUAACCCUGCUUUUGCU | SEQ ID NO: 87 |
| CAGUGAGUGAUUAUCAACCCUGCUUUUGCU | SEQ ID NO: 88 |
| CAAAAUCAUCAUCUCUUGUUUUUGUGUGUC | SEQ ID NO: 89 |
| AUUUGGAUUCAUUUUAAUCUCCUGCUUUUG | SEQ ID NO: 90 |
| AUUCCAUGCAAGUUUUUUCUCUUGUUUGGU | SEQ ID NO: 91 |
| AUUCCAUACAUGUUUCUUCUCUUGUUUGGU | SEQ ID NO: 92 |
| AUUCCAUACACGUUUUUUCUCUUGUCUGGU | SEQ ID NO: 93 |
| AUUCCAAACAUGUUUCUUCUCUUGUUUGGU | SEQ ID NO: 94 |
| AUUCCAAACAAGUUUUUCCUCUUGUUUGGU | SEQ ID NO: 95 |
| AUUCCAAACAAGUUUCUUCUCUUGUUUGGU | SEQ ID NO: 96 |
| AUGUCAUCUUGAAAACGCUCCGCUUCUGCU | SEQ ID NO: 97 |
| AUCCCAUACAUGUUUUUUCUCUUGUUUGGU | SEQ ID NO: 98 |
| AUCCAUUCAAGUGGUUUGCCUGCUUUUGCU | SEQ ID NO: 99 |
| AUCCAUUCAAAUGGUUUGCCUGCUUUUGCU | SEQ ID NO: 100 |
| AUCCAUUCAAAUGGUUUGCCUGCUUUCGCU | SEQ ID NO: 101 |
| AUCCAUUCAAAUGGUUUCGCUGCUUUCGCU | SEQ ID NO: 102 |
| AUAUCAAUUAGUUUUUUUGUUUUUUCUCGU | SEQ ID NO: 103 |
| ACCGAUAUCCCAUCUUCAUUUUCCCCUUGG | SEQ ID NO: 104 |
| AAUCACUAUAGUUUUUUUGUUUUUCUCCGU | SEQ ID NO: 105 |
| AACACGUAUCCAUAUUUCCCCUUGUUCGGU | SEQ ID NO: 106 |
| AAAAUCAUCAUCUCUUGUUUUUGUGUGUCU | SEQ ID NO: 107 |
| UCGACGUCGAUUUUCGGCGCGCGCCG | SEQ ID NO: 108 |
| GCGAUUUCUGACCGCUUUUUUGUCAG | SEQ ID NO: 109 |
| GUUGUGUUUUUACGGCGCCGUGCCG | SEQ ID NO: 110 |
| GUUGUGUACGGCGCCGTGCCG | SEQ ID NO: 111 |
| UUUUUCUUUUUGUGUGUCCG | SEQ ID NO: 112 |
| UUUUCCCCUGCUUUUGCUAA | SEQ ID NO: 113 |
| UUUAAUCUCCUGCUUUUGCU | SEQ ID NO: 114 |
| UUUAAUCUCCUGCUUUUGCU | SEQ ID NO: 115 |
| UUUAAACCCCUGCUUUUGCU | SEQ ID NO: 116 |
| UUGUACGCAUUUUUUCGCGU | SEQ ID NO: 117 |
| UUGUACGCAUUUUUUCGCGU | SEQ ID NO: 118 |
| UUGUACGCAUUUUUUCCCGU | SEQ ID NO: 119 |
| UUGGUUUUGUUGUUAAGCGU | SEQ ID NO: 120 |
| UUGAUCUGGUUGUUAAGCGU | SEQ ID NO: 121 |
| UUGAUCUGGUUGUUAAGCGU | SEQ ID NO: 122 |
| UUCUUUCUUUUUGUGUGCCC | SEQ ID NO: 123 |
| UUAUUAACCCUGCUUUUGCU | SEQ ID NO: 124 |
| UUAUCUACCCUGCUUUUGCU | SEQ ID NO: 125 |
| UUAUCAACCCUGCUUUUGCU | SEQ ID NO: 126 |
| UUACGGAUUCUCUGUUUGGU | SEQ ID NO: 127 |
| UUACAGAUUCUCUGUUUGGU | SEQ ID NO: 128 |
| UUAAAGGCUCCGCUUCUGCU | SEQ ID NO: 129 |
| UGUUUUUUCUCUUGUUUGGU | SEQ ID NO: 130 |
| UGUUUUUUCUCUUGUUUGGU | SEQ ID NO: 131 |
| UGUUUUUUCUCUUGUUUGGU | SEQ ID NO: 132 |
| UGAACUCCUGCUUUUGCUAA | SEQ ID NO: 133 |
| UCUUUCUUUUUGUGUGUCCG | SEQ ID NO: 134 |
| UCUCUUGUUUUUGUGUGUCU | SEQ ID NO: 135 |
| UCACGGAUUCUCUGUUUGGU | SEQ ID NO: 136 |
| UCACCGAUUCUCUGUUUGGU | SEQ ID NO: 137 |
| UCACAGAUUCUCUGUUUGGU | SEQ ID NO: 138 |
| UCAAGUUACUUUUUUUAUAC | SEQ ID NO: 139 |
| UCAAGUUACUUUUCUUAUAC | SEQ ID NO: 140 |
| UCAAACGUAUCGCUUCUGCU | SEQ ID NO: 141 |
| UCAAACGCACCGCUUCUGCU | SEQ ID NO: 142 |
| UAUUUUCCCCUGCUUUUGCU | SEQ ID NO: 143 |
| UAUACGCGUUUUUUUCGCGU | SEQ ID NO: 144 |
| UAGUACGCAUUUUUUCGCGU | SEQ ID NO: 145 |
| UAGUACGCAUUUUUUCGCGU | SEQ ID NO: 146 |
| GUUUUUUUGUUUUUUCUCGU | SEQ ID NO: 147 |
| GUUUUUUUGUUUUUCUCCGU | SEQ ID NO: 148 |
| GUGGUUUGCCUGCUUUUGCU | SEQ ID NO: 149 |
| GAUUUCCCCCUGCUUUUGCU | SEQ ID NO: 150 |
| GAUCAGUACCUGCUUUUGCU | SEQ ID NO: 151 |
| GAUCAGUACCUGCUUUCGCU | SEQ ID NO: 152 |
| GAAAAUGCUCUGCUUCUGCU | SEQ ID NO: 153 |
| GAAAAUAGCCAAUCUUAGCU | SEQ ID NO: 154 |
| GAAAACGCUCCGCUUCUGCU | SEQ ID NO: 155 |
| CUUAGCCAACUUUGUCUGGU | SEQ ID NO: 156 |
| CUUACCCAGCUUUGUUUGGU | SEQ ID NO: 157 |
| CUUACCCAGCUUUGUCUGGU | SEQ ID NO: 158 |
| CUUACCCAACUUUGUUUGGU | SEQ ID NO: 159 |
| CUUACCCAACUUUGUUUGGU | SEQ ID NO: 160 |
| CUUAAAGCUCCGCUUCUGCU | SEQ ID NO: 161 |
| CUGGUUUUGUUGUUAAGCGU | SEQ ID NO: 162 |
| CUCUUAAACUCUUGUCUGGU | SEQ ID NO: 163 |
| CUCAUCAUCUUUUAUGAUAC | SEQ ID NO: 164 |
| CGUUUUUUCUCUUGUCUGGU | SEQ ID NO: 165 |
| CAUCUUCAUUUUCCCCUUGG | SEQ ID NO: 166 |
| CAUAUUUCCCCUUGUUCGGU | SEQ ID NO: 167 |
| CAUAGCCAACUUUUUCUGGU | SEQ ID NO: 168 |
| AUUUUCCCCUGCUUUUGCUA | SEQ ID NO: 169 |
| AUUUUAAUCUCCUGCUUUUG | SEQ ID NO: 170 |
| AUUGGUUUUUUUGUCUUCGU | SEQ ID NO: 171 |
| AUUGGUUUGUUUUUCUUCGU | SEQ ID NO: 172 |
| AUGGUUUUUUUGUUAAGCGU | SEQ ID NO: 173 |
| AUGGUUUGCCUGCUUUUGCU | SEQ ID NO: 174 |
| AUGGUUUGCCUGCUUUCGCU | SEQ ID NO: 175 |
| AUGGUUUCGCUGCUUUCGCU | SEQ ID NO: 176 |
| AUCUUCUUUUUCCCCUUGGU | SEQ ID NO: 177 |
| AUCUUCAUUUUCCCCUUGGU | SEQ ID NO: 178 |
| AUCUCUUGUUUUUGUGUGUC | SEQ ID NO: 179 |
| AUCAUCAUCUUUUUUGAUAC | SEQ ID NO: 180 |
| AUAUUCAUUCUCCCCUUGGU | SEQ ID NO: 181 |
| AUAAUUGCGCUGCUUUCGCU | SEQ ID NO: 182 |
| AUAAUUGACCUGCUUUUCGU | SEQ ID NO: 183 |
| AUAAUUGACCUGCUUUCGCU | SEQ ID NO: 184 |
| AUAAUUGACCUGCUUUCGCU | SEQ ID NO: 185 |
| AGUUUCUUCUCUUGUUUGGU | SEQ ID NO: 186 |
| AGUUUUUCCUCUUGUUUGGU | SEQ ID NO: 187 |
| AGUUUCUUCUCUUGUUUGGU | SEQ ID NO: 188 |
| AGUUUCUUCUCUUGUUUGGU | SEQ ID NO: 189 |
| AGUCUCUUCUCUUGUUUGGU | SEQ ID NO: 190 |
| AAUUUUCCCCUGCUUUUGCU | SEQ ID NO: 191 |
| AAUGGUUUUUUUGUCUUCGU | SEQ ID NO: 192 |
| AAUGGUUUGUUUGUCUUCGU | SEQ ID NO: 193 |
| AAUGGUUUAUUUGUCUUCGU | SEQ ID NO: 194 |
| AAUCAGUACCUGCUUUUGCU | SEQ ID NO: 195 |
| AAUCAGUACCUGCUUUCGCU | SEQ ID NO: 196 |
| AAUAUCUACCUGCUUUUGCU | SEQ ID NO: 197 |
| AAUAUCUACCUGCUUUCGCU | SEQ ID NO: 198 |
| AACAUCUACCUGCUUUUGCU | SEQ ID NO: 199 |
| UUUUUUCGGCGGCCGCCG | SEQ ID NO: 200 |
| CGGCGGCCGCCGUUUUUU | SEQ ID NO: 201 |
| CCGUCUGUUGUUGGACUC | SEQ ID NO: 202 |
| CCGUCUGUUGUGUGACAG | SEQ ID NO: 203 |
| GGGGGGGUUGUGUGGGGG | SEQ ID NO: 204 |
| CGACUCUCUCUUCAGUUG | SEQ ID NO: 205 |
| CCGUCUGUUGUGUGACUC | SEQ ID NO: 206 |
| UUUUCGGCGGCCGCCG | SEQ ID NO: 207 |
| CGGCGGCCGCCGUUUU | SEQ ID NO: 208 |
| UUUUCGGCGCGCGCCG | SEQ ID NO: 209 |
| CGGCGCGCGCCGUUUU | SEQ ID NO: 210 |
| UUUGUUUGUCUUCGU | SEQ ID NO: 211 |
| UUUGUUGUUAAGCGU | SEQ ID NO: 212 |
| UUUGUUUUUUCUCGU | SEQ ID NO: 213 |
| UUUGUUUUUCUUCGU | SEQ ID NO: 214 |
| UUUGUUUUUUCUCGU | SEQ ID NO: 215 |
| UUUGUUUGUCUUCGU | SEQ ID NO: 216 |
| UUUGUUGUUAAGCGU | SEQ ID NO: 217 |
| UUUCUCUUGUUUGGU | SEQ ID NO: 218 |
| UUUCUCUUGUUUGGU | SEQ ID NO: 219 |
| UUUAUUUGUCUUCGU | SEQ ID NO: 220 |
| UUGUUUUUGUGUGUC | SEQ ID NO: 221 |
| UUGCCUGCUUUUGCU | SEQ ID NO: 222 |
| UUGCCUGCUUUCGCU | SEQ ID NO: 223 |
| UUCGCUGCUUUCGCU | SEQ ID NO: 224 |
| UUCCUCUUGUUUGGU | SEQ ID NO: 225 |
| UUCCCCUUGUUCGGU | SEQ ID NO: 226 |
| UUACUUUUUUUAUAC | SEQ ID NO: 227 |
| UUACUUUUUUUAUAC | SEQ ID NO: 228 |
| UGUUUUUGUGUGUCU | SEQ ID NO: 229 |
| UGCUCUGCUUCUGCU | SEQ ID NO: 230 |
| UGCGCUGCUUUCGCU | SEQ ID NO: 231 |
| UGACCUGCUUUUCGU | SEQ ID NO: 232 |
| UGACCUGCUUUCGCU | SEQ ID NO: 233 |
| UCUUUUUGUGUGCCC | SEQ ID NO: 234 |
| UUGCCUGCUUUUGCU | SEQ ID NO: 235 |
| UCCUGCUUUUGCUAA | SEQ ID NO: 236 |
| UACCCUGCUUUUGCU | SEQ ID NO: 237 |
| UCAUUUUCCCCUUGG | SEQ ID NO: 238 |
| UAGCCAAUCUUAGCU | SEQ ID NO: 239 |
| UACCCUGCUUUUGCU | SEQ ID NO: 240 |
| GUACCUGCUUUUGCU | SEQ ID NO: 241 |
| GUACCUGCUUUCGCU | SEQ ID NO: 242 |
| GGCUCCGCUUCUGCU | SEQ ID NO: 243 |
| GCGUUUUUUUCGCGU | SEQ ID NO: 244 |
| GAUUCUCUGUUUGGU | SEQ ID NO: 245 |
| CUUUUUGUGUGUCCG | SEQ ID NO: 246 |
| CUUUUUCCCCUUGGU | SEQ ID NO: 247 |
| CUUCUCUUGUUUGGU | SEQ ID NO: 248 |
| CUGGUUGUUAAGCGU | SEQ ID NO: 249 |
| CUACCUGCUUUCGCU | SEQ ID NO: 250 |
| CUACCUGCUUUCGCU | SEQ ID NO: 251 |
| CGUAUCGCUUCUGCU | SEQ ID NO: 252 |
| CGCUCCGCUUCUGCU | SEQ ID NO: 253 |
| CGCAUUUUUUCCCGU | SEQ ID NO: 254 |
| CGCAUUUUUUCCCGU | SEQ ID NO: 255 |
| CGCACCGCUUCUGCU | SEQ ID NO: 256 |
| CCCUGCUUUUGCUAA | SEQ ID NO: 257 |
| CCCCUGCUUUUGCUA | SEQ ID NO: 258 |
| CCAGCUUUGUUUGGU | SEQ ID NO: 259 |
| CCAGCUUUGUCUGGU | SEQ ID NO: 260 |
| CCAACUUUUUCUGGU | SEQ ID NO: 261 |
| CCAACUUUGUUUGGU | SEQ ID NO: 262 |
| CCAACUUUUUCUGGU | SEQ ID NO: 263 |
| CAUUUUCCCCUUGGU | SEQ ID NO: 264 |
| CAUUUUCCCCUUGGU | SEQ ID NO: 265 |
| CAUCUUUUAUGAUAC | SEQ ID NO: 266 |
| CAUCUUUUAUGAUAC | SEQ ID NO: 267 |
| CACCCUGCUUUUGCU | SEQ ID NO: 268 |
| AGCUCCGCUUCUGCU | SEQ ID NO: 269 |
| ACUCCUGCUUUUGCU | SEQ ID NO: 270 |
| ACCCCUGCUUUUGCU | SEQ ID NO: 271 |
| AAUCUCCUGCUUUUG | SEQ ID NO: 272 |
| AACCCUGCUUUUGCU | SEQ ID NO: 273 |
| AAACUCUUGUCUGGU | SEQ ID NO: 274 |
| UCGACGUCGAUUUU | SEQ ID NO: 275 |
| CUGUUGUGUGACAG | SEQ ID NO: 276 |
| GGGGUUUUGGGGG | SEQ ID NO: 277 |
| CCCCUUUUGGGGG | SEQ ID NO: 278 |
| GGGGGGGUUGUGU | SEQ ID NO: 279 |
| GUUUGUGUGGGG | SEQ ID NO: 280 |
| GGGGUUUUGGGG | SEQ ID NO: 281 |
| GGGGUUUUCCCC | SEQ ID NO: 282 |
| CGGCUUUUGCCG | SEQ ID NO: 283 |
| GAUCUUUUGAUC | SEQ ID NO: 284 |
| GUUGUGUGGGGG | SEQ ID NO: 285 |
| CUUCGGCUUCGG | SEQ ID NO: 286 |
| GGACUUUGGUCC | SEQ ID NO: 287 |
| GUCGGCGUUGAC | SEQ ID NO: 288 |
| GAUCUUUUCGGC | SEQ ID NO: 289 |
| AUAUUUUUCGGC | SEQ ID NO: 290 |
| GUUUGUGUGGG | SEQ ID NO: 291 |
| UUUUUUGGGGG | SEQ ID NO: 292 |
| UUUUUUAUAC | SEQ ID NO: 293 |
| UUUUUGAUAC | SEQ ID NO: 294 |
| UUUUUCUGGU | SEQ ID NO: 295 |
| UUUUUCUCGU | SEQ ID NO: 296 |
| UUUUUCGCGU | SEQ ID NO: 297 |
| UUUUUCCCGU | SEQ ID NO: 298 |
| UUUUCUCCGU | SEQ ID NO: 299 |
| UUUUCUCCGU | SEQ ID NO: 300 |
| UUUGUCUGGU | SEQ ID NO: 301 |
| UUUGUGUGUC | SEQ ID NO: 302 |
| UUUGUCUGGU | SEQ ID NO: 303 |
| UUUCUUAUAC | SEQ ID NO: 304 |
| UUUAUGAUAC | SEQ ID NO: 305 |
| UUGUGUGUCU | SEQ ID NO: 306 |
| UUGUGUGCCC | SEQ ID NO: 307 |
| UUGUCUUCGU | SEQ ID NO: 308 |
| UUCCCCUUGG | SEQ ID NO: 309 |
| UGUUAAGCGU | SEQ ID NO: 310 |
| UGUGUGUCCG | SEQ ID NO: 311 |
| UGCUUUUGCU | SEQ ID NO: 312 |
| UGCUUUUCGU | SEQ ID NO: 313 |
| UGCUUUCGCU | SEQ ID NO: 314 |
| UGCUUCUGCU | SEQ ID NO: 315 |
| UCUGUUUGGU | SEQ ID NO: 316 |
| UCCCCUUGGU | SEQ ID NO: 317 |
| GCUUUUGCUA | SEQ ID NO: 318 |
| CUUUUGCUAA | SEQ ID NO: 319 |
| CUUGUUUGGU | SEQ ID NO: 320 |
| CUUGUUCGGU | SEQ ID NO: 321 |
| CUUGUCUGGU | SEQ ID NO: 322 |
| CGCUUCUGCU | SEQ ID NO: 323 |
| CCUGCUUUUG | SEQ ID NO: 324 |
| AAUCUUAGCU | SEQ ID NO: 325 |
| UUUUUUUUUU | SEQ ID NO: 326 |
| UUUUUGGGGG | SEQ ID NO: 327 |
| GGGGGUUUUU | SEQ ID NO: 328 |
| UUGUGGGUCA | SEQ ID NO: 329 |
| UUUUUUUUU | |
| UUUUGGGGG | |
| UCCUUUCUU | |
| UUUUUUUU | |
| GUGUGUGU | |
| UUUUGGGG | |
| GGGGUUUU | |
| UUUUCGCG | |
| GUUGUGU | |
| UUUUUUU | |
| GUUUUGU | |
| GUUGUUU | |
| UUUGUGU | |
| UUUUUGU | |
| GUUUUUU | |
| GUUUUUG | |
| UUUUUU | |
| UUGUGU | |
| GUUGUG | |
| GUGUGU | |
| UUCGCG | |
| UUUUU | |
| UUGUG | |
| UGUGU | |
| GUUGU | |
| UUUU | |
| UUGU | |
| UGUG | |
| GUUG | |
| GUGU | |
| UGGU | |

In one embodiment the polymer includes at least one modified phosphate linkage provided as Formula I

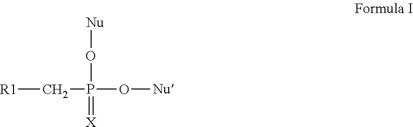

wherein R1 is hydrogen (H), COOR, OH, C1-C18 alkyl, C₆H₅, or (CH₂)ₘ—NH—R2, wherein R is H or methyl, butyl, methoxyethyl, pivaloyl oxymethyl, pivaloyl oxybenzyl, or S-pivaloyl thioethyl; R2 is H, C1-C18 alkyl, or C2-C18 acyl; and m is an integer from 1 to 17, inclusive; X is oxygen (O) or sulfur (S); and each of Nu and Nu′ independently is a nucleoside or nucleoside analog; with the proviso that if R1 is H, then X is S. As used herein, “C1-C18 alkyl” shall refer to any linear, cyclic, or branched alkyl group with 1 to 18 carbon atoms. In various embodiments the C1-C18 alkyl includes 1, 2, 3, 4, 5, 6, 7, 8, 9, 10, 11, 12, 13, 14, 15, 16, 17, or 18 carbon atoms. In one embodiment the C1-C18 alkyl is methyl. As used herein, “C2-C18 acyl” shall refer to any linear, cyclic, or branched acyl group with 2 to 18 carbon atoms. In various embodiments the C1-C18 acyl includes 2, 3, 4, 5, 6, 7, 8, 9, 10, 11, 12, 13, 14, 15, 16, 17, or 18 carbon atoms. As noted above, in one embodiment R1 is (CH₂)ₘ—NH—R2, wherein m is an integer from 1 to 17, inclusive. In various embodiments m is 1, 2, 3, 4, 5, 6, 7, 8, 9, 10, 11, 12, 13, 14, 15, 16, or 17.

In one embodiment the polymer includes at least one modified phosphate linkage provided as Formula II

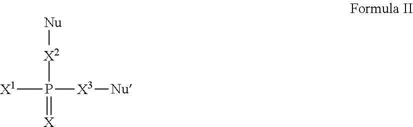

wherein X is O or S; X¹ is OH, SH, BH₃, OR3, or NHR3, wherein R3 is C1-C18 alkyl; each of X² and X³ independently is O, S, CH₂, or CF₂; and each of Nu and Nu′ independently is a nucleoside or nucleoside analog; with the proviso that (a) at least one of X, X², and X³ is not O or X¹ is not OH, (b) if X¹ is SH, then at least one of X, X², and X³ is not O, (c) if X and X² are O and if X¹ is OH, then X³ is not S and Nu is 3′Nu and Nu′ is 5′Nu′, and (d) if X′ is BH₃, then at least one of X, X², or X³ is S. As noted above with respect to Formula I, C1-C18 alkyl again refers to any linear, cyclic, or branched alkyl group with 1 to 18 carbon atoms.

It is to be noted that in one embodiment the immunostimulatory RNA motif is RNA. Even so, Nu or Nu' may in one embodiment represent a 5′ or 3′ terminal nucleoside of the immunostimulatory RNA motif. In this manner one or both termini of the immunostimulatory RNA motif may participate in a modified linkage as provided by Formula I or Formula II. Alternatively, no nucleoside of the immunostimulatory RNA motif can participate in a modified linkage as provided by Formula I or Formula II, such that all such modified linkages occur within the polymer but apart from the motif.

In one embodiment the polymer includes at least one nucleotide analog provided as Formula IIIA or Formula IIIB

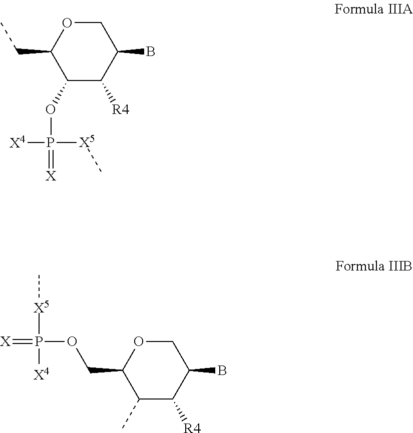

wherein R4 is H or OR, wherein R is H or C1-C18 acyl; B is a nucleobase, a modified nucleobase, or H; each of X and X⁵ independently is O or S; and X⁴ is OH, SH, methyl, or NHR5, wherein R5 is C1-C18 alkyl; each dashed line independently represents an optional bond to an adjacent unit, hydrogen, or an organic radical; with the proviso that at least one of X and X⁵ is not O or X⁴ is not OH. An organic radical refers to a group selected, for example, from hydroxyl, acylated hydroxy, phosphate, and a lipophilic residue as disclosed herein.

As with respect to Formulas I and H above, the C1-C18 alkyl in Formula IIIA and Formula IIIB again refers to a linear, cyclic, or branched alkyl group with 1 to 18 carbon atoms. It will be appreciated that the sugar moiety of the nucleotide analog of Formula IIIA or Formula IIIB has a six-membered ring. It will also be appreciated that when B is hydrogen (H), the nucleotide analog of Formula MA or Formula IIIB is an abasic nucleotide analog, i.e., it has no nucleobase but is a unit in the backbone of the polymer nonetheless. In one embodiment the nucleotide analog of Formula IIIA or Formula IIIB is excluded from the immunostimulatory RNA motif.

In one embodiment according to this aspect of the invention in Formula IIIA or Formula IIIB R4 is OH, X⁴ is SH, and each of X and X⁵ is O.

As mentioned above, RNA is a polymer of ribonucleotides joined through 3′-5′ phosphodiester linkages. Units of the polymer of the invention can also be joined through 3′-5′ phosphodiester linkages. However, the invention also encompasses polymers having unusual internucleotide linkages, including specifically 5′-5′, 3′-3′, 2′-2′, 2′-3′, and 2′-5′ internucleotide linkages. In one embodiment such unusual linkages are excluded from the immunostimulatory RNA motif, even though one or more of such linkages may occur elsewhere within the polymer. For polymers having free ends, inclusion of one 3′-3′ internucleotide linkage can result in a polymer having two free 5′ ends. Conversely, for polymers having free ends, inclusion of one 5′-5′ internucleotide linkage can result in a polymer having two free 3′ ends.

An immunostimulatory composition of this invention can contain two or more immunostimulatory RNA motifs which can be linked through a branching unit. The internucleotide linkages can be 3′-5′, 5′-5′, 3′-3′, 2′-2′, 2′-3, or 2′-5′ linkages. Thereby, the nomenclature 2′-5′ is chosen according to the carbon atom of ribose. However, if unnatural sugar moieties are employed, such as ring-expanded sugar analogs (e.g., hexanose, cylohexene or pyranose) or bi- or tricyclic sugar analogs, then this nomenclature changes according to the nomenclature of the monomer. The unusual internucleotide linkage can be a phosphodiester linkage, but it can alternatively be modified as phosphorothioate or any other modified linkage as described herein. Formula IV shows a general structure for branched RNA oligomers and modified oligoribonucleotide analogs of the invention via a nucleotidic branching unit. Thereby Nu₁, Nu₂, and Nu₃ can be linked through 3′-5′, 5′-5′, 3′-3′, 2′-2′, 2′-3′, or 2′-5′-linkages. Branching of RNA oligomers can also involve the use of non-nucleotidic linkers and abasic spacers. In one embodiment, Nu₁, Nu₂, and Nu₃ represent identical or different immunostimulatory RNA motifs. In another embodiment, Nu₁, Nu₂, and Nu₃ comprises at least one immunostimulatory RNA motif and at least one immunostimulatory CpG DNA motif.

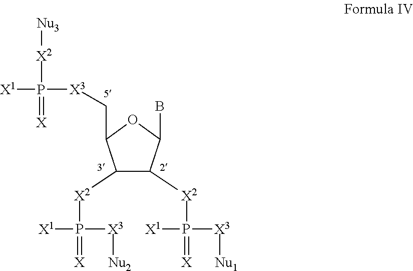

The modified oligoribonucleotide analog may contain a doubler or trebler unit (Glen Research, Sterling, Va.), in particular those modified oligoribonucleotide analogs with a 3′-3′ linkage. A doubler unit in one embodiment can be based on 1,3-bis-[5-(4,4′-dimethoxytrityloxy)pentylamido]propyl-2-[(2-cyanoethyl)-(N,N-diisopropyl)]-phosphoramidite. A trebler unit in one embodiment can be based on incorporation of Tris-2,2,2-[3-(4,4′-dimethoxytrityloxy)propyloxymethyl]ethyl-[(2-cyanoethyl)-(N,N-diisopropyl)]-phosphoramidite. Branching of the modified oligoiribonucleotide analogs by multiple doubler, trebler, or other multiplier units leads to dendrimers which are a further embodiment of this invention. Branched modified oligoribonucleotide analogs may lead to crosslinking of receptors for immunostimulatory RNA such as TLR3, TLR7, and TLR8, with distinct immune effects compared to non-branched forms of the analogs. In addition, the synthesis of branched or otherwise multimeric analogs may stabilize RNA against degradation and may enable weak or partially effective RNA sequences to exert a therapeutically useful level of immune activity. The modified oligoribonucleotide analogs may also contain linker units resulting from peptide modifying reagents or oligonucleotide modifying reagents (Glen Research). Furthermore, the modified oligoribonucleotide analogs may contain one or more natural or unnatural amino acid residues which are connected to the polymer by peptide (amide) linkages.

The 3′-5′, 5′-5′, 3′-3′, 2′-2′, 2′-3′, and 2′-5′ internucleotide linkages can be direct or indirect. Direct linkages in this context refers to a phosphate or modified phosphate linkage as disclosed herein, without an intervening linker moiety. An intervening linker moiety is an organic moiety distinct from a phosphate or modified phosphate linkage as disclosed herein, which can include, for example, polyethylene glycol, triethylene glycol, hexaethylene glycol, dSpacer (i.e., an abasic deoxynucleotide), doubler unit, or trebler unit.

An immunostimulatory composition of the invention can in one embodiment include one or more modified nucleobases outside of the immunostimulatory RNA motif. Specific embodiments of these modified nucleobases include hypoxanthine, inosine, 8-oxo-adenine, 7-substituted derivatives thereof, dihydrouracil, pseudouracil, 2-thiouracil, 4-thiouracil, 5-aminouracil, 5-(C₁-C₆)-alkyluracil, 5-methyluracil, 5-(C₂-C₆)-alkenyluracil, 5-(C₂-C₆)-alkynyluracil, 5-(hydroxymethyl)uracil, 5-chlorouracil, 5-fluorouracil, 5-bromouracil, 5-hydroxycytosine, 5-(C₁-C₆)-alkylcytosine, 5-methylcytosine, 5-(C₂-C₆)-alkenylcytosine, 5-(C₂-C₆)-alkynylcytosine, 5-chlorocytosine, 5-fluorocytosine, 5-bromocytosine, N²-dimethylguanine, 7-deazaguanine, 8-azaguanine, 7-deaza-7-substituted guanine, 7-deaza-7-(C2-C6)alkynylguanine, 7-deaza-8-substituted guanine, 8-hydroxyguanine, 6-thioguanine, 8-oxoguanine, 2-aminopurine, 2-amino-6-chloropurine, 2,4-diaminopurine, 2,6-diaminopurine, 8-azapurine, substituted 7-deazapurine, 7-deaza-7-substituted purine, 7-deaza-8-substituted purine, and hydrogen (abasic residue). These modified nucleobases and their corresponding ribonucleosides are available from commercial suppliers.

It has been discovered according to the invention that a 2′ O-alkyl sugar modification inside or outside of the immunostimulatory RNA motif reduces or inhibits the activity of the immunostimulatory composition. In certain embodiments this modification renders the immunostimulatory composition inactive. In one embodiment a composition of the invention excludes a 2′ O-alkyl sugar modification inside or outside of the immunostimulatory RNA motif.

It has also been discovered according to the invention that inclusion of a 2′ fluoro modification with a phosphodiester linkage within the immunostimulatory RNA motif of an immunostimulatory composition of the invention retains immunostimulatory activity, whereas inclusion of a 2′ fluoro modification with a phosphorothioate linkage within the immunostimulatory RNA motif of an immunostimulatory composition of the invention renders the immunostimulatory composition inactive. In one embodiment a composition of the invention includes a 2′ fluoro modification with a phosphodiester linkage within the immunostimulatory RNA motif. In one embodiment a composition of the invention excludes a 2′ fluoro modification with a phosphorothioate linkage within the immunostimulatory RNA motif.

An immunostimulatory composition of the invention can in one embodiment include one or modified U nucleobases, which may occur anywhere in the polymer. Specific embodiments of such modified U nucleobases include dihydrouracil, pseudouracil, 2-thiouracil, 4-thiouracil, 5-aminouracil, 5-(C₁-C₆)-alkyluracil, 5-methyluracil, 5-(C₂-C₆)-alkenyluracil, 5-(C₂-C₆)-alkynyluracil, 5-(hydroxymethyl)uracil, 5-chlorouracil, 5-fluorouracil, and 5-bromouracil. These modified U nucleobases and their corresponding ribonucleosides are available from commercial suppliers.

An immunostimulatory composition of the invention can in one embodiment include one or more modified G nucleobases, which may occur anywhere in the polymer. Specific embodiments of such modified G nucleobases include N²-dimethylguanine, 7-deazaguanine, 8-azaguanine, 7-deaza-7-substituted guanine, 7-deaza-7-(C2-C6)alkynylguanine, 7-deaza-8-substituted guanine, 8-hydroxyguanine, 6-thioguanine, and 8-oxoguanine. In one embodiment the modified G nucleobase is 8-hydroxyguanine. These modified G nucleobases and their corresponding ribonucleosides are available from commercial suppliers.

In certain embodiments at least one β-ribose unit may be replaced by β-D-deoxyribose or a modified sugar unit, wherein the modified sugar unit is for example selected from β-D-ribose, α-D-ribose, β-L-ribose (as in ‘Spiegelmers’), α-L-ribose, 2′-amino-2′-deoxyribose, 2′-fluoro-2′-deoxyribose, 2′-O-(C1-C6)alkyl-ribose, preferably 2′-O-(C1-C6)alkyl-ribose is 2′-O-methylribose, 2′-O-(C2-C6)alkenyl-ribose, 2′-[O-(C1-C6)alkyl-O-(C1-C6)alkyl]-ribose, LNA and α-LNA (Nielsen P et al. (2002) Chemistry-A European Journal 8:712-22), β-D-xylo-furanose, α-arabinofuranose, 2′-fluoro arabinofuranose, and carbocyclic and/or open-chain sugar analogs (described, for example, in Vandendriessche et al. (1993) Tetrahedron 49:7223) and/or bicyclosugar analogs (described, for example, in Tarkov Met al. (1993) Helv Chim Acta 76:481).

Modified oligoribonucleotide analogs in which at least one ribose unit is replaced by 1,5-anhydrohexitol (Bouvere B et al. (1997) Nucleosides Nucleotides 16:973-6) or by D-Altritol (Allart B et al. (1999) Chemistry-A European Journal 5:2424-31) are also embodiments of this invention. In another embodiment, the modified oligoribonucleotide analog comprises at least one β-D-ribopyranosyl unit (“pyranosyl-RNA”; Pitsch S et al. (2003) Helv Chim Acta 86:4270-363). Alternatively, other ring-expanded or ring-condensed sugar analogs may replace ribose.

In another embodiment, at least one hydroxy group, preferably the 2′-hydroxy group, of the ribose unit is protected as a pro-drug, which is cleaved in vivo to release the oligomer with unprotected ribose. Known pro-drugs of ribose are e.g. the corresponding valinates (Kong L et al. (2003) Antivir Chem Chemother 14:263-70), formates (Repta A et al. (1975) J Pharm Sci 64:392-6), or isopropyl ethers (Winkelmann E et al. (1988) Arzneimittelforschung 38:1545-8).

In specific embodiments the immunostimulatory compositions of the invention exclude a deoxycytidine-deoxyguanosine (dCdG; CG DNA) dinucleotide. In particular, the immunostimulatory compositions of the invention in one embodiment exclude a CpG DNA dinucleotide, i.e., a 5′-deoxycytidine-deoxyguanosine-3′ dinucleotide in which cytosine is unmethylated and the deoxycytidine and deoxyguanosine are linked together by a phosphate bond. CpG dinucleotides, in the context of certain flanking sequences resulting in a “CpG motif”, are believed to be stimulatory ligands for TLR9. Such immunostimulatory CpG motifs were originally described principally in connection with certain types of naturally occurring and synthetic forms of DNA, and they generally are of the form X₁X₂CpGX₃X₄, wherein X₁, X₂, X₃, and X₄ are nucleotides and X₁X₂ preferably represents GG, GA, GT, AT, or AA, and X₃X₄ preferably represents TT or CT. See, for example, U.S. Pat. No. 6,239,116.

In certain embodiments the modified oligoribonucleotide analog is conjugated to another entity to provide a conjugate. As used herein a conjugate refers to a combination of any two or more entities bound to one another by any physicochemical means, including hydrophobic interaction and covalent coupling.

In another embodiment, the modified oligoribonucleotide analog may be conjugated to a small molecular weight ligand which is recognized by an immunomodulatory receptor. This receptor is preferably a member of the TLR family, such as TLR2, TLR3, TLR4, TLR7, TLR8, or TLR9. The small molecular weight ligands are mimics of the natural ligands for these receptors. Examples include but are not limited to R-848 (Resiquimod), R-837 (Imiquimod; ALDARA™, 3M Pharmaceuticals), 7-deaza-guanosine, 7-thia-8-oxo-guansosine, and 7-allyl-8-oxo-guansosine (Loxoribine) which stimulate either TLR7 or TLR8. D-Glucopyranose derivatives, such as 3D-MPL (TLR4 ligand), may also be conjugated to the modified oligoribonucleotide analogs. Pam3-Cys is an example of a TLR2 ligand which can be conjugated to modified oligoribonucleotide analogs. Oligodeoxynucleotides containing CpG motifs are TLR9 ligands, and these can also be conjugated to modified oligoribonucleotide analogs of the invention. In one embodiment, at least one oligodeoxynucleotide comprising a CpG motif effective for stimulating TLR9 signaling is conjugated to a modified oligoribonucleotide analog of this invention. Conjugation of ligands for different TLRs into one molecule may lead to multimerisation of receptors which results in enhanced immune stimulation or a different immunostimulatory profile from that resulting from any single such ligand.

In certain embodiments the polymer is covalently linked to a lipophilic moiety. The lipophilic moiety generally will occur at one or more ends of a polymer having free ends, although in certain embodiments the lipophilic moiety can occur elsewhere along the polymer and thus does not require the polymer have a free end. In one embodiment the polymer has a 3′ end and the lipophilic moiety is covalently linked to the 3′ end. The lipophilic group in general can be a cholesteryl, a modified cholesteryl, a cholesterol derivative, a reduced cholesterol, a substituted cholesterol, cholestan, C16 alkyl chain, a bile acid, cholic acid, taurocholic acid, deoxycholate, oleyl litocholic acid, oleoyl cholenic acid, a glycolipid, a phospholipid, a sphingolipid, an isoprenoid, such as steroids, vitamins, such as vitamin E, saturated fatty acids, unsaturated fatty acids, fatty acid esters, such as triglycerides, pyrenes, porphyrines, Texaphyrine, adamantane, acridines, biotin, coumarin, fluorescein, rhodamine, Texas-Red, digoxygenin, dimethoxytrityl, t-butyldimethylsilyl, t-butyldiphenylsilyl, cyanine dyes (e.g. Cy3 or Cy5), Hoechst 33258 dye, psoralen, or ibuprofen. In certain embodiments the lipophilic moiety is chosen from cholesteryl, palmityl, and fatty acyl. In one embodiment the lipohilic moiety is cholesteryl. It is believed that inclusion of one or more of such lipophilic moieties in the polymers of the invention confers upon them yet additional stability against degradation by nucleases. Where there are two or more lipophilic moieties in a single polymer of the invention, each lipophilic moiety can be selected independently of any other.

In an embodiment the polymer is linked to cholesterol either at the 3′ end or at the 5′ end. In certain embodiments the cholesterol may be linked via a phosphodiester or phosphorothioate linkage.

In one embodiment the polymer is linked to the lipophilic moiety hexadecylglycerol. In one embodiment the hexadecylglycerol is linked at the 3′ end. In one embodiment the hexadecylglycerol is linked at the 5′ end. It has been discovered according to the invention that hexadecylglycerol, when linked to either the 3′ end or the 5′ end of the polymer, confers markedly increased activity in the presence of DOTAP (N-[1-(2,3-Dioleoyloxy)propyl]-N,N,N-trimethylammonium methyl-sulfate). In one embodiment the polymer linked to hexadecylglycerol is in the presence of DOTAP.

In one embodiment the lipophilic group is attached to a 2′-position of a nucleotide or nucleotide analog of the polymer. A lipophilic group can alternatively or in addition be linked to the heterocyclic nucleobase of a nucleotide or nucleotide analog of the polymer. The lipophilic moiety can be covalently linked to the polymer via any suitable direct or indirect linkage. In one embodiment the linkage is direct and is an ester or an amide. In various embodiments the lipophilic group is optionally linked via a phosphorothioate, phosphodiester, or methyl phosphonate linkage to an end of the oligonucleotide. In one embodiment the linkage is indirect and includes a spacer moiety, for example one or more abasic nucleotide residues, oligoethyleneglycol, such as triethyleneglycol (spacer 9) or hexaethyleneglycol (spacer 18), or an alkane-diol, such as butanediol. In various embodiments the spacer moiety is optionally linked to the oligomer via at least one phosphorothioate, phosphodiester, or methyl phosphonate linkage.

In one embodiment the lipophilic group is attached to a 3′-position of a nucleotide or nucleotide analog of the polymer. The lipophilic moiety can be covalently linked to the polymer via any suitable direct or indirect linkage. In one embodiment the linkage is direct and is an ester or an amide. In various embodiments the lipophilic group is optionally linked via a phosphorothioate, phosphodiester, or methyl phosphonate linkage to an end of the oligoribonucleotide. In one embodiment the linkage is indirect and includes a spacer moiety, for example one or more abasic nucleotide residues, oligoethyleneglycol, such as triethyleneglycol (spacer 9) or hexaethyleneglycol (spacer 18), or an alkane-diol, such as butanediol. In various embodiments the spacer moiety is optionally linked to the oligomer via at least one phosphorothioate, phosphodiester, or methyl phosphonate linkage.

In one embodiment the modified oligoribonucleotide analog of the invention is advantageously combined with a cationic lipid. Cationic lipids are believed to assist in trafficking of the modified oligoribonucleotide analog into the endosomal compartment, where TLR7 and TLR8 (as well as TLR9) are found. In one embodiment the cationic lipid is DOTAP (N-[1-(2,3-dioleoyloxy)propy-1]-N,N,N-trimethylammonium methyl-sulfate). DOTAP is believed to transport modified oligoribonucleotide analog into cells and specifically traffic to the endosomal compartment, where it can release the modified oligoribonucleotide analog in a pH-dependent fashion. Once in the endosomal compartment, the immunostimulatory RNA motif can interact with certain intracellular TLRs, triggering TLR-mediated signal transduction pathways involved in generating an immune response. Other agents with similar properties including trafficking to the endosomal compartment can be used in place of or in addition to DOTAP.

In an embodiment the polymer is linked to a lipophilic moiety and is in the presence of DOTAP.

In one embodiment the composition of the invention further includes a polyG sequence covalently linked to at least one end of the polymer, wherein each polyG sequence independently includes 4-12 consecutive guanosine nucleosides selected from the group consisting of guanosine ribonucleoside, guanosine deoxyribonucleoside, and any combination thereof. The polyG sequence in one embodiment includes stabilized internucleotide phosphate linkages, e.g., phosphorothioate linkages. PolyG sequences can confer a number of biological and physicochemical properties, including stabilization against nucleases, enhanced uptake by cells, inhibition of certain cytokines, and formation of secondary or intermolecular structure involving so-called G-tetrads. In one embodiment the polymer has a 3′ end and the polyG sequence is covalently linked to the 3′ end. The polyG sequence can be covalently linked to the polymer via any suitable direct or indirect linkage, usually via a backbone linkage.

The compositions of the invention encompass polymers with and without secondary or higher order structure. For example, the polymer in one embodiment includes a sequence of nucleosides, nucleoside analogs, or a combination of nucleosides and nucleoside analogs capable of forming secondary structure provided by at least two adjacent hydrogen-bonded base pairs. In one embodiment the at least two adjacent hydrogen-bonded base pairs involve two sets of at least 3 consecutive bases. The consecutive nature of involved bases is thermodynamically advantageous for forming a so-called clamp. However, consecutive bases may not be required, particularly where there is high GC content and/or extended sequence. Typically there will be 3, 4, 5, 6, 7, 8, 9, 10, 11, 12, 13, 14, 15, or 16 base pairs. A hydrogen-bonded base pair in one embodiment can be classical Watson-Crick base pair, i.e., G-C, A-U, or A-T. In other embodiments a hydrogen-bonded base pair can be a non-classical base pair, such as G-U, G-G, G-A, or U-U. In yet other embodiments a hydrogen-bonded base pair can be a Hoogstein or other base pair.

In one embodiment the secondary structure is a stem-loop secondary structure. A stem-loop or hairpin secondary structure can arise through intramolecular hydrogen-bonded base pairing between complementary or at least partially complementary sequences. The complementary or at least partially complementary sequences represent perfect or interrupted inverted repeat sequences, respectively. For example, a polymer having a base sequence provided by 5′-X₁-X₂-X₃ . . . X₃′-X₂′-X₁′-3′, wherein each of X₁ and X₁′, X₂ and X₂′, and X₃ and X₃′ can form a hydrogen-bonded base pair, may include a perfect or interrupted inverted repeat and has the potential to fold on itself and form a stem-loop secondary structure. It will be appreciated that a polymer having a base sequence provided by 5′-X₁-X₂-X₃ . . . X₃′-X₂′-X₁′-3′, wherein each of X₁ and X₁′, X₂ and X₂′, and X₃ and X₃′ can form a hydrogen-bonded base pair, also has the potential to form intermolecular complexes through intermolecular hydrogen-bonded base pairs. Where there are two or more inverted repeats, individual polymers can also interact to form not only dimeric intermolecular complexes but also higher-order intermolecular complexes or structures. Persons skilled in the art will recognize that conditions and/or sequences can be selected so as to favor formation of one type of secondary structure over another.

In one embodiment the modified oligoribonucleotide analogs of the invention are in the form of covalently closed, dumbbell-shaped molecules with both primary and secondary structure. As described below, in one embodiment such cyclic oligoribonucleotide analogs include two single-stranded loops connected by an intervening double-stranded segment. In one embodiment at least one single-stranded loop includes an immunostimulatory RNA motif of the invention. Other covalently closed, dumbbell-shaped molecules of the invention include chimeric DNA:RNA molecules in which, for example, the double-stranded segment is at least partially DNA (e.g., either homodimeric dsDNA or heterodimeric DNA:RNA) and at least one single-stranded loop includes an immunostimulatory RNA motif of the invention. Such embodiment represents one type of chimeric DNA:RNA construct of the invention, where such constructs in general are encompassed by the present invention. In one embodiment at least one single-stranded loop includes an immunostimulatory RNA motif of the invention and at least one single-stranded loop includes an immunostimulatory CpG DNA motif Such embodiment represents one type of conjugate of the invention between a modified oligoribonucleotide analog of the invention and a CpG DNA, where such conjugates in general are encompassed by the present invention. In one embodiment the at least one single-stranded loop including an immunostimulatory RNA motif of the invention and the at least one single-stranded loop including an immunostimulatory CpG DNA motif are separate single-stranded loops, for example, on opposite ends of a dumbbell-shaped molecule.

For use in the instant invention the polymers of the invention can be synthesized de novo using or adapted from any of a number of procedures well known in the art. For example, the β-cyanoethyl phosphoramidite method (Beaucage S L et al. (1981) Tetrahedron Lett 22:1859); nucleoside H-phosphonate method (Garegg P et al. (1986) Tetrahedron Lett 27:4051-4; Froehler B C et al. (1986) Nucl Acid Res 14:5399-407; Garegg P et al. (1986) Tetrahedron Lett 27:4055-8; Gaffney B L et al. (1988) Tetrahedron Lett 29:2619-22). These chemistries can be performed by a variety of automated nucleic acid synthesizers available in the market. Additional synthesis methods useful according to the instant invention are disclosed in Uhlmann E et al. (1990) Chem Rev 90:544-84, and Goodchild J (1990) Bioconjugate Chem 1:165.

Oligoribonucleotide synthesis can be performed either in solution or on a solid-phase support. In solution, block coupling reactions (dimers, trimers, tetramers, etc.) are preferred, while solid-phase synthesis is preferably performed in a stepwise process using monomeric building blocks. Different chemistries, such as the phosphotriester method, H-phosphonate method, and phosphoramidite method, have been described (Eckstein F (1991) Oligonucleotides and Analogues, A Practical Approach, IRL Press, Oxford). While in the phosphotriester method the reactive phosphorus group is in the oxidation state +V, the more reactive Phosphor +III derivatives are used in the coupling reactions according to the phosphoramidite and H-phosphonate approaches. In the latter two approaches, phosphorus is oxidized after the coupling step to yield the stable P(V) derivatives. If the oxidizer is iodine/water/base, then phosphodiesters are obtained after deprotection. In contrast, if the oxidizer is a sulfurizing agent, such as Beaucage's Reagent, then phosphorothioates are obtained after deprotection.

An efficient method for oligoribonucleotide synthesis is the combination of solid-support synthesis using phosphoramidite chemistry as originally described for oligodeoxynucleotides by Matteucci and Caruthers. Matteucci M D et al. (1981) J Am Chem Soc 103:3185.

Synthesis of oligoribonucleotides is similar to oligodeoxynucleotides, with the difference that the 2′-hydroxy group present in oligoribonucleotides must be protected by a suitable hydroxy protecting group. The monomers can be protected e.g. by 2′-O-t-butyldimethylsilyl (TBDMS) group in the RNA monomeric building blocks. However, RNA synthesis using monomers containing the 2′-O-TriisopropylsilylOxyMethyl (TOM) group (TOM-Protecting-Group™) has been reported to yield higher coupling efficiency, because the TOM-Protecting-Group exhibits lower steric hindrance than the TBDMS group. While the TBDMS protecting group is removed using fluoride, fast deprotection is achieved for the TOM group using methylamine in ethanol/water at room temperature. In oligo(ribo)nucleotide synthesis, chain elongation from 3′- to 5′-end is preferred, which is achieved by coupling of a ribonucleotide unit having a 3′-phosphor (III) group or its activated derivative to a free 5′-hydroxy group of another nucleotide unit.

Synthesis can be conveniently performed using automated an DNA/RNA synthesizer. Thereby, synthesis cycles as recommended by the suppliers of the synthesizers can be used. For ribonucleoside phosphoramidite monomers, coupling times are longer (e.g., 400 sec) as compared to deoxynucleoside monomers. As solid support, 500 to 1000 Å controlled pore glass (CPG) support or organic polymer support, such as primer support PS200 (Amersham), can be used. The solid support usually contains the first nucleoside, such as 5′-O-Dimethoxytrityl-N-6-benzoyladenosine, attached via its 3′-end. After cleavage of the 5′-O-Dimethoxytrityl-group with trichloroacetic acid, chain elongation is achieved using e.g. 5′-O-Dimethoxytrityl-N-protected-2′-O-tert butyldimethylsilyl-nucleoside-3′-O-phosphoramidites. After successive repetitive cycles, the completed oligoribonucleotide is cleaved from the support and deprotected by treatment with concentrated ammonia/ethanol (3:1, v:v) for 24 hours at 30° C. The TBDMS blocking group is finally cleaved off using triethylamine/HF. The crude oligoribonucleotides can be purified by ion exchange high pressure liquid chromatography (HPLC), ion-pair reverse phase HPLC, or polyacrylamide gel electrophoresis (PAGE) and characterized by mass spectrometry.

Synthesis of 5′-conjugates is straightforward by coupling a phosphoramidite of the molecule to be ligated to the 5′-hydroxy group of the terminal nucleotide in solid-phase synthesis. A variety of phosphoramidite derivatives of such ligands, such as cholesterol, acridine, biotin, psoralene, ethyleneglycol, or aminoalkyl residues are commercially available. Alternatively, aminoalkyl functions can be introduced during solid-phase synthesis which allow post-synthesis derivatization by activated conjugate molecules, such as active esters, isothiocynates, or iodo-acetamides.

Synthesis of 3′-end conjugates is usually achieved by using the correspondingly modified solid supports, such as e.g. commercially available cholesterol-derivatized solid supports. Conjugation can however also be done at internucleotide linkages, nucleobases or at the ribose residues, such as at the 2′-postion of ribose.

For cyclic oligoribonucleotides, the elongation of the oligonucleotide chain can be carried out on Nucleotide PS solid support (Glen Research) using standard phosphoramidite chemistry. The cyclization reaction is then carried out on the solid support using a phosphotriester coupling procedure (Alazzouzi et al. (1997) Nucleosides Nucleotides 16:1513-14). On final deprotection with ammonium hydroxide, virtually the only product which comes into solution is the desired cyclic oligonucleotide.

Cyclic oligoribonucleotide analogs of the invention inlcude closed circular forms of RNA and can include single-stranded RNA with or without double-stranded RNA. For example, in one embodiment the cyclic oligoribouncleotide analog includes double-stranded RNA and takes on a dumbbell conformation with two single-stranded loops connected by an intervening double-stranded segment. Covalently closed, dumbbell-shaped CpG oligodeoxynucleotides have been described in U.S. Pat. No. 6,849,725. In another embodiment the cyclic oligoribonucleotide analog includes double-stranded RNA and takes on a conformation with three or more single-stranded loops connected by intervening double-stranded segments. In one embodiment an immunostimulatory RNA motif is located in one or more single-stranded segments.

The modified oligoribonucleotide analogs of the invention are useful, alone or in combination with other agents, as adjuvants. An adjuvant as used herein refers to a substance other than an antigen that enhances immune cell activation in response to an antigen, e.g., a humoral and/or cellular immune response. Adjuvants promote the accumulation and/or activation of accessory cells to enhance antigen-specific immune responses. Adjuvants are used to enhance the efficacy of vaccines, i.e., antigen-containing compositions used to induce protective immunity against the antigen.

Adjuvants in general include adjuvants that create a depot effect, immune-stimulating adjuvants, and adjuvants that create a depot effect and stimulate the immune system. An adjuvant that creates a depot effect as used herein is an adjuvant that causes the antigen to be slowly released in the body, thus prolonging the exposure of immune cells to the antigen. This class of adjuvants includes but is not limited to alum (e.g., aluminum hydroxide, aluminum phosphate); emulsion-based formulations including mineral oil, non-mineral oil, water-in-oil or oil-in-water-in oil emulsion, oil-in-water emulsions such as Seppic ISA series of Montanide adjuvants (e.g., Montanide ISA 720; AirLiquide, Paris, France); MF-59 (a squalene-in-water emulsion stabilized with Span 85 and Tween 80; Chiron Corporation, Emeryville, Calif.); and PROVAX (an oil-in-water emulsion containing a stabilizing detergent and a micelle-forming agent; IDEC Pharmaceuticals Corporation, San Diego, Calif.).

An immune-stimulating adjuvant is an adjuvant that causes activation of a cell of the immune system. It may, for instance, cause an immune cell to produce and secrete cytokines. This class of adjuvants includes but is not limited to saponins purified from the bark of the Q. saponaria tree, such as QS21 (a glycolipid that elutes in the 21st peak with HPLC fractionation; Aquila Biopharmaceuticals, Inc., Worcester, Mass.); poly[di(carboxylatophenoxy)phosphazene (PCPP polymer; Virus Research Institute, USA); derivatives of lipopolysaccharides such as monophosphoryl lipid A (MPL; Ribi ImmunoChem Research, Inc., Hamilton, Mont.), muramyl dipeptide (MDP; Ribi) andthreonyl-muramyl dipeptide (t-MDP; Ribi); OM-174 (a glucosamine disaccharide related to lipid A; OM Pharma SA, Meyrin, Switzerland); and Leishmania elongation factor (a purified Leishmania protein; Corixa Corporation, Seattle, Wash.). This class of adjuvants also includes CpG DNA.

Adjuvants that create a depot effect and stimulate the immune system are those compounds which have both of the above-identified functions. This class of adjuvants includes but is not limited to ISCOMS (immunostimulating complexes which contain mixed saponins, lipids and form virus-sized particles with pores that can hold antigen; CSL, Melbourne, Australia); SB-AS2 (SmithKline Beecham adjuvant system #2 which is an oil-in-water emulsion containing MPL and QS21: SmithKline Beecham Biologicals [SBB], Rixensart, Belgium); SB-AS4 (SmithKline Beecham adjuvant system #4 which contains alum and MPL; SBB, Belgium); non-ionic block copolymers that form micelles such as CRL 1005 (these contain a linear chain of hydrophobic polyoxypropylene flanked by chains of polyoxyethylene; Vaxcel, Inc., Norcross, Ga.); and Syntex Adjuvant Formulation (SAF, an oil-in-water emulsion containing Tween 80 and a nonionic block copolymer; Syntex Chemicals, Inc., Boulder, Colo.).

Thus the invention in one aspect provides an adjuvant that includes a modified oligoribonucleotide analog of the invention, by itself. In another embodiment the invention provides an adjuvant that includes a modified oligoribonucleotide analog of the invention and at least one other adjuvant (a combination adjuvant). The other adjuvant can include an adjuvant that creates a depot effect, an immune-stimulating adjuvant, an adjuvant that creates a depot effect and stimulates the immune system, and any combination thereof. In one embodiment the modified oligoribonucleotide analog of the invention and at least one other adjuvant are covalently linked to one another. A combination adjuvant according to the invention may exhibit a synergistic immunostimulatory effect compared to the sum of effects of the modified oligoribonucleotide analog alone and the at least one other adjuvant alone. Additionally or alternatively, a combination adjuvant according to the invention may exhibit an altered immunostimulatory profile compared to that of either the modified oligoribonucleotide analog alone or the at least one other adjuvant alone. For example, the combination adjuvant may provide a more balanced form of Th1/Th2 immunostimulation in one embodiment, or it may provide a more skewed form of Th1/Th2 immunostimulation in another embodiment. Those skilled in the art will recognize how to select individual components to promote a desired type of immunostimulation, e.g, more balanced or more skewed with respect to Th1 and Th2 character. Th1 and Th2 are described further below.

Also provided is a composition that includes a modified oligoribonucleotide analog of the invention plus another adjuvant, wherein the other adjuvant is a cytokine. In one embodiment the composition is a conjugate of the modified oligoribonucleotide analog of the invention and the cytokine.

Cytokines are soluble proteins and glycoproteins produced by many types of cells that mediate inflammatory and immune reactions. Cytokines mediate communication between cells of the immune system, acting locally as well as systemically to recruit cells and to regulate their function and proliferation. Categories of cytokines include mediators and regulators of innate immunity, mediators and regulators of adaptive immunity, and stimulators of hematopoiesis. Included among cytokines are interleukins (e.g., IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, and interleukins 19-32 (IL-19-IL-32), among others), chemokines (e.g., IP-10, RANTES, MIP-1α, MIP-1β, MIP-3α, MCP-1, MCP-2, MCP-3, MCP-4, eotaxin, I-TAC, and BCA-1, among others), as well as other cytokines including type 1 interferons (e.g., IFN-α and IFN-β), type 2 interferon (e.g., IFN-γ), tumor necrosis factor-alpha (TNF-α), transforming growth factor-beta (TGF-β), and various colony stimulating factors (CSFs), including GM-CSF, G-CSF, and M-CSF.

Also provided is a composition that includes a modified oligoribonucleotide analog of the invention plus another adjuvant, wherein the other adjuvant is an immunostimulatory CpG nucleic acid. In one embodiment the composition is a conjugate of the modified ribonucleotide analog of the invention and the CpG nucleic acid.

An immunostimulatory CpG nucleic acid as used herein refers to a natural or synthetic DNA sequence that includes a CpG motif and that stimulates activation or proliferation of cells of the immune system. Immunostimulatory CpG nucleic acids have been described in a number of issued patents, published patent applications, and other publications, including U.S. Pat. Nos. 6,194,388; 6,207,646; 6,214,806; 6,218,371; 6,239,116; and 6,339,068. In one embodiment the immunostimulatory CpG nucleic acid is a CpG oligodeoxynucleotide (CpG ODN) 6-100 nucleotides long. In one embodiment the immunostimulatory CpG nucleic acid is a CpG oligodeoxynucleotide (CpG ODN) 8-40 nucleotides long.

Immunostimulatory CpG nucleic acids include different classes of CpG nucleic acids. One class is potent for activating B cells but is relatively weak in inducing IFN-α and NK cell activation; this class has been termed the B class. The B class CpG nucleic acids typically are fully stabilized and include an unmethylated CpG dinucleotide within certain preferred base contexts. See, e.g., U.S. Pat. Nos. 6,194,388; 6,207,646; 6,214,806; 6,218,371; 6,239,116; and 6,339,068. Another class is potent for inducing IFN-α and NK cell activation but is relatively weak at stimulating B cells; this class has been termed the A class. The A class CpG nucleic acids typically have a palindromic phosphodiester CpG dinucleotide-containing sequence of at least 6 nucleotides and a stabilized poly-G sequences at either or both the 5′ and 3′ ends. See, for example, published international patent application WO 01/22990. Yet another class of CpG nucleic acids activates B cells and NK cells and induces IFN-α; this class has been termed the C class. The C class CpG nucleic acids, as first characterized, typically are fully stabilized, include a B class-type sequence and a GC-rich palindrome or near-palindrome. This class has been described in published U.S. patent application 2003/0148976, the entire contents of which are incorporated herein by reference.

Immunostimulatory CpG nucleic acids also include so-called soft and semi-soft CpG nucleic acids, as disclosed in published U.S. patent application 2003/0148976, the entire contents of which is incorportated herein by reference. Such soft and semi-soft immunostimulatory CpG nucleic acids incorporate a combination of nuclease-resistant and nuclease-sensitive internucleotide linkages, wherein the different types of linkages are positioned according to certain rules.

A soft oligonucleotide is an immunostimulatory oligonucleotide having a partially stabilized backbone, in which phosphodiester or phosphodiester-like internucleotide linkages occur only within and immediately adjacent to at least one internal pyrimidine-purine dinucleotide (YZ). Preferably YZ is YG, a pyrimidine-guanosine (YG) dinucleotide. The at least one internal YZ dinucleotide itself has a phosphodiester or phosphodiester-like internucleotide linkage. A phosphodiester or phosphodiester-like internucleotide linkage occurring immediately adjacent to the at least one internal YZ dinucleotide can be 5′, 3′, or both 5′ and 3′ to the at least one internal YZ dinucleotide.

In particular, phosphodiester or phosphodiester-like internucleotide linkages involve “internal dinucleotides”. An internal dinucleotide in general shall mean any pair of adjacent nucleotides connected by an internucleotide linkage, in which neither nucleotide in the pair of nucleotides is a terminal nucleotide, i.e., neither nucleotide in the pair of nucleotides is a nucleotide defining a 5′ or 3′ end of the oligonucleotide. Thus a linear oligonucleotide that is n nucleotides long has a total of n-1 dinucleotides and only n-3 internal dinucleotides. Each internucleotide linkage in an internal dinucleotide is an internal internucleotide linkage. Thus a linear oligonucleotide that is n nucleotides long has a total of n-1 internucleotide linkages and only n-3 internal internucleotide linkages. The strategically placed phosphodiester or phosphodiester-like internucleotide linkages, therefore, refer to phosphodiester or phosphodiester-like internucleotide linkages positioned between any pair of nucleotides in the nucleic acid sequence. In some embodiments the phosphodiester or phosphodiester-like internucleotide linkages are not positioned between either pair of nucleotides closest to the 5′ or 3′ end.

Preferably a phosphodiester or phosphodiester-like internucleotide linkage occurring immediately adjacent to the at least one internal YZ dinucleotide is itself an internal internucleotide linkage. Thus for a sequence N₁ YZ N₂, wherein N₁ and N₂ are each, independent of the other, any single nucleotide, the YZ dinucleotide has a phosphodiester or phosphodiester-like internucleotide linkage, and in addition (a) N₁ and Y are linked by a phosphodiester or phosphodiester-like internucleotide linkage when N₁ is an internal nucleotide, (b) Z and N₂ are linked by a phosphodiester or phosphodiester-like internucleotide linkage when N₂ is an internal nucleotide, or (c) N₁ and Y are linked by a phosphodiester or phosphodiester-like internucleotide linkage when N₁ is an internal nucleotide and Z and N₂ are linked by a phosphodiester or phosphodiester-like internucleotide linkage when N₂ is an internal nucleotide.

Soft oligonucleotides are believed to be relatively susceptible to nuclease cleavage compared to completely stabilized oligonucleotides. Without meaning to be bound to a particular theory or mechanism, it is believed that soft oligonucleotides of the invention are cleavable to fragments with reduced or no immunostimulatory activity relative to full-length soft oligonucleotides. Incorporation of at least one nuclease-sensitive internucleotide linkage, particularly near the middle of the oligonucleotide, is believed to provide an “off switch” which alters the pharmacokinetics of the oligonucleotide so as to reduce the duration of maximal immunostimulatory activity of the oligonucleotide. This can be of particular value in tissues and in clinical applications in which it is desirable to avoid injury related to chronic local inflammation or immunostimulation, e.g., the kidney.

A semi-soft oligonucleotide is an immunostimulatory oligonucleotide having a partially stabilized backbone, in which phosphodiester or phosphodiester-like internucleotide linkages occur only within at least one internal pyrimidine-purine (YZ) dinucleotide. Semi-soft oligonucleotides generally possess increased immunostimulatory potency relative to corresponding fully stabilized immunostimulatory oligonucleotides. Due to the greater potency of semi-soft oligonucleotides, semi-soft oligonucleotides may be used, in some instances, at lower effective concentations and have lower effective doses than conventional fully stabilized immunostimulatory oligonucleotides in order to achieve a desired biological effect.

It is believed that the foregoing properties of semi-soft oligonucleotides generally increase with increasing “dose” of phosphodiester or phosphodiester-like internucleotide linkages involving internal YZ dinucleotides. Thus it is believed, for example, that generally for a given oligonucleotide sequence with five internal YZ dinucleotides, an oligonucleotide with five internal phosphodiester or phosphodiester-like YZ internucleotide linkages is more immunostimulatory than an oligonucleotide with four internal phosphodiester or phosphodiester-like YG internucleotide linkages, which in turn is more immunostimulatory than an oligonucleotide with three internal phosphodiester or phosphodiester-like YZ internucleotide linkages, which in turn is more immunostimulatory than an oligonucleotide with two internal phosphodiester or phosphodiester-like YZ internucleotide linkages, which in turn is more immunostimulatory than an oligonucleotide with one internal phosphodiester or phosphodiester-like YZ internucleotide linkage. Importantly, inclusion of even one internal phosphodiester or phosphodiester-like YZ internucleotide linkage is believed to be advantageous over no internal phosphodiester or phosphodiester-like YZ internucleotide linkage. In addition to the number of phosphodiester or phosphodiester-like internucleotide linkages, the position along the length of the nucleic acid can also affect potency.

A phosphodiester internucleotide linkage is the type of linkage characteristic of nucleic acids found in nature. A phosphodiester internucleotide linkage includes a phosphorus atom flanked by two bridging oxygen atoms and bound also by two additional oxygen atoms, one charged and the other uncharged. Phosphodiester internucleotide linkage is particularly preferred when it is important to reduce the tissue half-life of the oligonucleotide.

A phosphodiester-like intemucleotide linkage is a phosphorus-containing bridging group that is chemically and/or diastereomerically similar to phosphodiester. Measures of similarity to phosphodiester include susceptibility to nuclease digestion and ability to activate RNAse H. Thus for example phosphodiester, but not phosphorothioate, oligonucleotides are susceptible to nuclease digestion, while both phosphodiester and phosphorothioate oligonucleotides activate RNAse H. In a preferred embodiment the phosphodiester-like internucleotide linkage is boranophosphate (or equivalently, boranophosphonate) linkage. U.S. Pat. No. 5,177,198; U.S. Pat. No. 5,859,231; U.S. Pat. No. 6,160,109; U.S. Pat. No. 6,207,819; Sergueev et al., (1998) J Am Chem Soc 120:9417-27. In another preferred embodiment the phosphodiester-like intemucleotide linkage is diasteromerically pure Rp phosphorothioate. It is believed that diasteromerically pure Rp phosphorothioate is more susceptible to nuclease digestion and is better at activating RNAse H than mixed or diastereomerically pure Sp phosphorothioate. Stereoisomers of CpG oligonucleotides are the subject of published international patent application WO 00/06588.

Also provided is a composition that includes a modified ribonucleotide analog of the invention plus another adjuvant, wherein the other adjuvant is a lipopeptide such as

Pam3Cys, a cationic polysaccharide such as chitosan, or a cationic peptide such as protamine. In one embodiment the composition is a conjugate of the modified ribonucleotide analog of the invention and the other adjuvant.

The compositions of the invention can optionally include an antigen. An “antigen” as used herein refers to any molecule capable of being recognized by a T-cell antigen receptor or B-cell antigen receptor. The term broadly includes any type of molecule which is recognized by a host immune system as being foreign. Antigens generally include but are not limited to cells, cell extracts, proteins, polypeptides, peptides, polysaccharides, polysaccharide conjugates, peptide and non-peptide mimics of polysaccharides and other molecules, small molecules, lipids, glycolipids, polysaccharides, carbohydrates, viruses and viral extracts, and multicellular organisms such as parasites, and allergens. With respect to antigens that are proteins, polypeptides, or peptides, such antigens can include nucleic acid molecules encoding such antigens. Antigens more specifically include, but are not limited to, cancer antigens, which include cancer cells and molecules expressed in or on cancer cells; microbial antigens, which include microbes and molecules expressed in or on microbes; and allergens.

The invention in one aspect provides a use of a modified oligoribonucleotide analog of the invention for the preparation of a medicament for vaccinating a subject.

The invention in one aspect provides a method for preparing a vaccine. The method includes the step of placing a modified oligoribonucleotide analog of the invention in intimate association with an antigen and, optionally, a pharmaceutically acceptable carrier.

In various embodiments the antigen is a microbial antigen, a cancer antigen, or an allergen. A “microbial antigen” as used herein is an antigen of a microorganism and includes but is not limited to viruses, bacteria, parasites, and fungi. Such antigens include the intact microorganism as well as natural isolates and fragments or derivatives thereof and also synthetic compounds which are identical to or similar to natural microorganism antigens and induce an immune response specific for that microorganism. A compound is similar to a natural microorganism antigen if it induces an immune response (humoral and/or cellular) to a natural microorganism antigen. Such antigens are used routinely in the art and are well known to those of ordinary skill in the art.

Viruses are small infectious agents which generally contain a nucleic acid core and a protein coat, but are not independently living organisms. Viruses can also take the form of infectious nucleic acids lacking a protein. A virus cannot survive in the absence of a living cell within which it can replicate. Viruses enter specific living cells either by endocytosis or direct injection of DNA (phage) and multiply, causing disease. The multiplied virus can then be released and infect additional cells. Some viruses are DNA-containing viruses and others are RNA-containing viruses. In some aspects, the invention also intends to treat diseases in which prions are implicated in disease progression such as for example bovine spongiform encephalopathy (i.e., mad cow disease, BSE) or scrapie infection in animals, or Creutzfeldt-Jakob disease in humans.

Viruses include, but are not limited to, enteroviruses (including, but not limited to, viruses that the family picornaviridae, such as polio virus, coxsackie virus, echo virus), rotaviruses, adenovirus, hepatitis virus. Specific examples of viruses that have been found in humans include but are not limited to: Retroviridae (e.g., human immunodeficiency viruses, such as HIV-1 (also referred to as HTLV-III, LAV or HTLV-III/LAV, or HIV-III; and other isolates, such as HIV-LP; Picornaviridae (e.g., polio viruses, hepatitis A virus; enteroviruses, human Coxsackie viruses, rhinoviruses, echoviruses); Calciviridae (e.g., strains that cause gastroenteritis); Togaviridae (e.g., equine encephalitis viruses, rubella viruses); Flaviviridae (e.g., dengue viruses, encephalitis viruses, yellow fever viruses); Coronaviridae (e.g., coronaviruses); Rhabdoviridae (e.g., vesicular stomatitis viruses, rabies viruses); Filoviridae (e.g., ebola viruses); Paramyxoviridae (e.g., parainfluenza viruses, mumps virus, measles virus, respiratory syncytial virus); Orthomyxoviridae (e.g., influenza viruses); Bunyaviridae (e.g., Hantaan viruses, bunya viruses, phleboviruses and Nairo viruses); Arenaviridae (hemorrhagic fever viruses); Reoviridae (e.g., reoviruses, orbiviurses and rotaviruses); Birnaviridae; Hepadnaviridae (Hepatitis B virus); Parvoviridae (parvoviruses); Papovaviridae (papillomaviruses, polyoma viruses); Adenoviridae (most adenoviruses); Herpesviridae (herpes simplex virus (HSV) 1 and 2, varicella zoster virus, cytomegalovirus (CMV)); Poxviridae (variola viruses, vaccinia viruses, pox viruses); Iridoviridae (e.g., African swine fever virus); and unclassified viruses (e.g., the etiological agents of spongiform encephalopathies, the agent of delta hepatitis (thought to be a defective satellite of hepatitis B virus), the agents of non-A, non-B hepatitis (class 1=internally transmitted; class 2=parenterally transmitted (i.e., Hepatitis C); Norwalk and related viruses, and astroviruses).

Bacteria are unicellular organisms which multiply asexually by binary fission. They are classified and named based on their morphology, staining reactions, nutrition and metabolic requirements, antigenic structure, chemical composition, and genetic homology. Bacteria can be classified into three groups based on their morphological forms, spherical (coccus), straight-rod (bacillus) and curved or spiral rod (vibrio, campylobacter, spirillum, and spirochaete). Bacteria are also more commonly characterized based on their staining reactions into two classes of organisms, gram-positive and gram-negative. Gram refers to the method of staining which is commonly performed in microbiology labs. Gram-positive organisms retain the stain following the staining procedure and appear a deep violet color. Gram-negative organisms do not retain the stain but take up the counter-stain and thus appear pink.

Infectious bacteria include, but are not limited to, gram negative and gram positive bacteria. Gram positive bacteria include, but are not limited to Pasteurella species, Staphylococci species, and Streptococcus species. Gram negative bacteria include, but are not limited to, Escherichia coli, Pseudomonas species, and Salmonella species. Specific examples of infectious bacteria include but are not limited to: Helicobacter pyloris, Borrelia burgdorferi, Legionella pneumophilia, Mycobacteria sps (e.g., M. tuberculosis, M. avium, M. intracellulare, M. kansasii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic species), Streptococcus pneumoniae, pathogenic Campylobacter sp., Enterococcus sp., Haemophilus influenzae, Bacillus anthracia, Corynebacterium diphtheriae, Corynebacterium sp., Erysipelothrix rhusiopathiae, Clostridium perfringens, Clostridium tetani, Enterobacter aerogenes, Klebsiella pneumoniae, Pasturella multocida, Bacteroides sp., Fusobacterium nucleatum, Streptobacillus moniliformis, Treponema pallidum, Treponema pertenue, Leptospira, Rickettsia, and Actinomyces israelli.

Parasites are organisms which depend upon other organisms in order to survive and thus must enter, or infect, another organism to continue their life cycle. The infected organism, i.e., the host, provides both nutrition and habitat to the parasite. Although in its broadest sense the term parasite can include all infectious agents (i.e., bacteria, viruses, fungi, protozoa and helminths), generally speaking, the term is used to refer solely to protozoa, helminths, and ectoparasitic arthropods (e.g., ticks, mites, etc.). Protozoa are single-celled organisms which can replicate both intracellularly and extracellularly, particularly in the blood, intestinal tract or the extracellular matrix of tissues. Helminths are multicellular organisms which almost always are extracellular (an exception being Trichinella spp.). Helminths normally require exit from a primary host and transmission into a secondary host in order to replicate. In contrast to these aforementioned classes, ectoparasitic arthropods form a parasitic relationship with the external surface of the host body.

Parasites include intracellular parasites and obligate intracellular parasites. Examples of parasites include but are not limited to Plasmodium falciparum, Plasmodium ovale, Plasmodium malariae, Plasmdodium vivax, Plasmodium knowlesi, Babesia microti, Babesia divergens, Trypanosoma cruzi, Toxoplasma gondii, Trichinella spiralis, Leishmania major, Leishmania donovani, Leishmania braziliensis, Leishmania tropica, Trypanosoma gambiense, Trypanosoma rhodesiense and Schistosoma mansoni.

Fungi are eukaryotic organisms, only a few of which cause infection in vertebrate mammals. Because fungi are eukaryotic organisms, they differ significantly from prokaryotic bacteria in size, structural organization, life cycle and mechanism of multiplication. Fungi are classified generally based on morphological features, modes of reproduction and culture characteristics. Although fungi can cause different types of disease in subjects, such as respiratory allergies following inhalation of fungal antigens, fungal intoxication due to ingestion of toxic substances, such as Amanita phalloides toxin and phallotoxin produced by poisonous mushrooms and aflatoxins, produced by aspergillus species, not all fungi cause infectious disease.

Infectious fungi can cause systemic or superficial infections. Primary systemic infection can occur in normal healthy subjects, and opportunistic infections are most frequently found in immunocompromised subjects. The most common fungal agents causing primary systemic infection include Blastomyces, Coccidioides, and Histoplasma. Common fungi causing opportunistic infection in immunocompromised or immunosuppressed subjects include, but are not limited to, Candida albicans, Cryptococcus neoformans, and various Aspergillus species. Systemic fungal infections are invasive infections of the internal organs. The organism usually enters the body through the lungs, gastrointestinal tract, or intravenous catheters. These types of infections can be caused by primary pathogenic fungi or opportunistic fungi.

Superficial fungal infections involve growth of fungi on an external surface without invasion of internal tissues. Typical superficial fungal infections include cutaneous fungal infections involving skin, hair, or nails.

Diseases associated with fungal infection include aspergillosis, blastomycosis, candidiasis, chromoblastomycosis, coccidioidomycosis, cryptococcosis, fungal eye infections, fungal hair, nail, and skin infections, histoplasmosis, lobomycosis, mycetoma, otomycosis, paracoccidioidomycosis, disseminated Penicillium marneffei, phaeohyphomycosis, rhinosporidioisis, sporotrichosis, and zygomycosis.

Other medically relevant microorganisms have been described extensively in the literature, e.g., see C. G. A Thomas, Medical Microbiology, Bailliere Tindall, Great Britain 1983, the entire contents of which is hereby incorporated by reference. Each of the foregoing lists is illustrative and is not intended to be limiting.

As used herein, the terms “cancer antigen” and “tumor antigen” are used interchangeably to refer to a compound, such as a peptide, protein, or glycoprotein, which is associated with a tumor or cancer cell and which is capable of provoking an immune response when expressed on the surface of an antigen-presenting cell in the context of a major histocompatibility complex (MHC) molecule. Cancer antigens which are differentially expressed by cancer cells and can thereby be exploited in order to target cancer cells. Cancer antigens are antigens which can potentially stimulate apparently tumor-specific immune responses. Some of these antigens are encoded, although not necessarily expressed, by normal cells. These antigens can be characterized as those which are normally silent (i.e., not expressed) in normal cells, those that are expressed only at certain stages of differentiation, and those that are temporally expressed such as embryonic and fetal antigens. Other cancer antigens are encoded by mutant cellular genes, such as oncogenes (e.g., activated ras oncogene), suppressor genes (e.g., mutant p53), fusion proteins resulting from internal deletions or chromosomal translocations. Still other cancer antigens can be encoded by viral genes such as those carried on RNA and DNA tumor viruses.

Cancer antigens can be prepared from cancer cells either by preparing crude extracts of cancer cells, for example, as described in Cohen P A et al. (1994) Cancer Res 54:1055-8, by partially purifying the antigens, by recombinant technology, or by de novo synthesis of known antigens. Cancer antigens include but are not limited to antigens that are recombinantly expressed, an immunogenic portion of, or a whole tumor or cancer or cell thereof. Such antigens can be isolated or prepared recombinantly or by any other means known in the art.

Examples of tumor antigens include MAGE, MART-1/Melan-A, gp100, dipeptidyl peptidase IV (DPPIV), adenosine deaminase-binding protein (ADAbp), cyclophilin b, colorectal associated antigen (CRC)—C017-1A/GA733, carcinoembryonic antigen (CEA) and its immunogenic epitopes CAP-1 and CAP-2, etv6, aml1, prostate specific antigen (PSA) and its immunogenic epitopes PSA-1, PSA-2, and PSA-3, prostate-specific membrane antigen (PSMA), T-cell receptor/CD3-zeta chain, MAGE-family of tumor antigens (e.g., MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-C5), GAGE-family of tumor antigens (e.g., GAGE-1, GAGE-2, GAGE-3, GAGE-4, GAGE-5, GAGE-6, GAGE-7, GAGE-8, GAGE-9), BAGE, RAGE, LAGE-1, NAG, GnT-V, MUM-1, CDK4, tyrosinase, p53, MUC family, HER2/neu, p21ras, RCAS1, α-fetoprotein, E-cadherin, α-catenin, β-catenin and γ-catenin, p120ctn, gp100^{Pmel117}, PRAME, NY-ESO-1, cdc27, adenomatous polyposis coli protein (APC), fodrin, Connexin 37, Ig-idiotype, p15, gp75, GM2 and GD2 gangliosides, viral products such as human papillomavirus proteins, Smad family of tumor antigens, lmp-1, P1A, EBV-encoded nuclear antigen (EBNA)-1, brain glycogen phosphorylase, SSX-1, SSX-2 (HOM-MEL-40), SSX-1, SSX-4, SSX-5, SCP-1 and CT-7, and c-erbB-2. This list is not meant to be limiting.

An “allergen” as used herein is a molecule capable of provoking an immune response characterized by production of IgE. An allergen is also a substance that can induce an allergic or asthmatic response in a susceptible subject. Thus, in the context of this invention, the term allergen means a specific type of antigen which can trigger an allergic response which is mediated by IgE antibody.

The list of allergens is enormous and can include pollens, insect venoms, animal dander dust, fungal spores and drugs (e.g., penicillin). Examples of natural animal and plant allergens include proteins specific to the following genuses: Canis (Canis familiaris); Dermatophagoides (e.g., Dermatophagoides farinae); Felis (Fells domesticus); Ambrosia (Ambrosia artemiisfolia; Lolium (e.g., Lolium perenne and Lolium multiflorum); Cryptomeria (Cryptomeria japonica); Alternaria (Alternaria alternata); Alder; Alnus (Alnus gultinosa); Betula (Betula verrucosa); Quercus (Quercus alba); Olea (Olea europa); Artemisia (Artemisia vulgaris); Plantago (e.g., Plantago lanceolata); Parietaria (e.g., Parietaria officinalis and Parietaria judaica); Blattella (e.g., Blattella germanica); Apis (e.g., Apis multiflorum); Cupressus (e.g., Cupressus sempervirens, Cupressus arizonica and Cupressus macrocarpa); Juniperus (e.g., Juniperus sabinoides, Juniperus virginiana, Juniperus communis, and Juniperus ashei); Thuya (e.g., Thuya orientalis); Chamaecyparis (e.g., Chamaecyparis obtusa); Periplaneta (e.g., Periplaneta americana); Agropyron (e.g., Agropyron repens); Secale (e.g., Secale cereale); Triticum (e.g., Triticum aestivum); Dactylis (e.g., Dactylis glomerata); Festuca (e.g., Festuca elatior); Poa (e.g., Poa pratensis and Poa compressa); Avena (e.g., Avena sativa); Holcus (e.g., Holcus lanatus); Anthoxanthum (e.g., Anthoxanthum odoratum); Arrhenatherum (e.g., Arrhenatherum elatius); Agrostis (e.g., Agrostis alba); Phleum (e.g., Phleum pratense); Phalaris (e.g., Phalaris arundinacea); Paspalum (e.g., Paspalum notatum); Sorghum (e.g., Sorghum halepensis); and Bromus (e.g., Bromus inermis).

The invention in one aspect provides a conjugate of a modified oligoribonucleotide analog of the invention and an antigen. In one embodiment the modified oligoribonucleotide analog of the invention is covalently linked to the antigen. The covalent linkage between the modified oligoribonucleotide analog and the antigen can be any suitable type of covalent linkage, provided the modified oligoribonucleotide analog and the antigen when so joined retain measurable functional activity of each individual component. In one embodiment the covalent linkage is direct. In another embodiment the covalent linkage is indirect, e.g., through a linker moiety. The covalently linked modified oligoribonucleotide analog and antigen may be processed within a cell to release one from the other. In this way delivery to a cell of either component may be enhanced compared to its delivery if administered as a separate preparatation or separate component.

In one embodiment the antigen is an antigen per se, i.e., it is a preformed antigen. In another embodiment the antigen is in the form of a nucleic acid encoding a proteinaceous antigen. In one embodiment the nucleic acid encoding the antigen is operatively linked to a gene expression sequence which directs the expression of the antigen nucleic acid within a eukaryotic cell. The gene expression sequence is any regulatory nucleotide sequence, such as a promoter sequence or promoter-enhancer combination, which facilitates the efficient transcription and translation of the antigen nucleic acid to which it is operatively linked. Such gene expression sequence can be isologous or heterologous in origin with respect to a gene that encodes the antigen. The gene expression sequence may be, for example, a mammalian or viral promoter, such as a constitutive or inducible promoter. Constitutive mammalian promoters include, but are not limited to, the promoters for the following genes: hypoxanthine phosphoribosyl transferase (HPRT), adenosine deaminase, pyruvate kinase, β-actin, and other constitutive promoters. Exemplary viral promoters which function constitutively in eukaryotic cells include, for example, promoters from the cytomegalovirus (CMV), simian virus (e.g., SV40), papilloma virus, adenovirus, human immunodeficiency virus (HIV), Rous sarcoma virus, the long terminal repeats (LTR) of Moloney leukemia virus and other retroviruses, and the thymidine kinase promoter of herpes simplex virus. Other constitutive promoters are known to those of ordinary skill in the art. The promoters useful as gene expression sequences of the invention also include inducible promoters. Inducible promoters are expressed in the presence of an inducing agent. For example, the metallothionein promoter is induced to promote transcription and translation in the presence of certain metal ions. Other inducible promoters are known to those of ordinary skill in the art.

In one aspect the invention provides a pharmaceutical composition which includes a composition of the invention, in association with a delivery vehicle. In various embodiments the delivery vehicle can be chosen from a cationic lipid, a liposome, a cochleate, a virosome, an immune-stimulating complex (ISCOM), a microparticle, a microsphere, a nanosphere, a unilamellar vesicle (LUV), a multilamellar vesicle, an oil-in-water emulsion, a water-in-oil emulsion, an emulsome, and a polycationic peptide, and, optionally, a pharmaceutically acceptable carrier. Pharmaceutically acceptable carriers are discussed below. The pharmaceutical composition of the invention optionally can further include an antigen. The composition of the invention, along with the antigen when present, is brought into physical association with the delivery vehicle using any suitable method. The immunostimulatory composition can be contained within the delivery vehicle, or it can be present on or in association with a solvent-exposed surface of the delivery vehicle. In one embodiment the polymer is present on or in association with a solvent-exposed surface of the delivery vehicle, and the antigen, if present, is contained within the delivery vehicle. In another embodiment both the polymer and the antigen are present on or in association with a solvent-exposed surface of the delivery vehicle. In yet another embodiment the antigen is present on or in association with a solvent-exposed surface of the delivery vehicle, and the polymer is contained within the delivery vehicle. In yet another embodiment both the polymer and the antigen, if antigen is included, are contained within the delivery vehicle.

The invention also provides methods for use of the immunostimulatory compositions of the invention. In one aspect the invention provides a method of activating an immune cell. The method according to this aspect of the invention includes the step of contacting an immune cell, in vitro or in vivo, with an effective amount of a composition of the invention, to activate the immune cell. The composition of the invention can optionally include an antigen. An “immune cell” as used herein refers to any bone marrow-derived cell that can participate in an innate or adaptive immune response. Cells of the immune system include, without limitation, dendritic cells (DC), natural killer (NK) cells, monocytes, macrophages, granulocytes, B lymphocytes, plasma cells, T lymphocytes, and precursor cells thereof.

As used herein, the term “effective amount” refers to that amount of a substance that is necessary or sufficient to bring about a desired biological effect. An effective amount can but need not be limited to an amount administered in a single administration.

As used herein, the term “activate an immune cell” refers to inducing an immune cell to enter an activated state that is associated with an immune response. As used herein, the term “immune response” refers to any aspect of an innate or adaptive immune response that reflects activation of an immune cell to proliferate, to perform an effector immune function, or to produce a gene product involved in an immune response. Gene products involved in an immune response can include secreted products (e.g., antibodies, cytokines, and chemokines) as well as intracellular and cell surface molecules characteristic of immune function (e.g., certain cluster of differentiation (CD) antigens, transcription factors, and gene transcripts). The term “immune response” can be applied to a single cell or to a population of cells.

Production of cytokines can be assessed by any of several methods well known in the art, including biological response assays, enzyme-linked immunosorbent assay (ELISA), intracellular fluorescence-activated cell sorting (FACS) analysis, and reverse transcriptase/polymerase chain reaction (RT-PCR).

In one embodiment the immune response is a Th1-like immune response. A Th1-like immune response can include expression of any of certain cytokines and chemokines, including IFN-α, IFN-γ, TNF-α, IL-12, IL-18, IP-10, and any combination thereof, that are characteristically associated with a Th1 immune response. In some embodiments the Th1-like immune response can include suppression of certain Th2-associated cytokines, including IL-4, IL-5, and IL-13. The Th1-like immune response can include expression of certain antibody isotypes, including (in the mouse) IgG2a, with or without suppression of certain Th2-associated antibody isotypes, including IgE and (in the mouse) IgG1. In one embodiment a Th1-like immune response is a Th1 response.

A Th2-like immune response can include expression of any of certain cytokines and chemokines, including IL-4, IL-5, IL-10, IL-13, and any combination thereof, that are characteristically associated with a Th2 immune response. In some embodiments the Th2-like immune response can include suppression of certain Th1-associated cytokines. The Th2-like immune response can include expression of certain antibody isotypes, including IgE and (in the mouse) IgG1, with or without suppression of certain Th1-associated antibody isotypes, including (in the mouse) IgG2a. In one embodiment a Th2-like immune response is a Th2 response.

Thus in one embodiment the invention provides a method for inducing a Th1-like immune response in a subject. Inducing a Th1-like immune response includes augmenting a Th1-like immune response. The method includes the step of administering to a subject an effective amount of a modified oligoribonucleotide analog of the invention to induce a Th1-like immune response in the subject.

In one embodiment the invention provides a method for suppressing a Th2-like immune response in a subject. The method includes the step of administering to a subject an effective amount of a modified oligoribonucleotide analog of the invention to suppress a Th2-like immune response in the subject. Such method may find particular use in the treatment of subjects having or at risk of having a condition characterized by an immune response with predominant Th2 character. Such conditions include, without limitation, allergy and asthma.

In one embodiment the immune response involves upregulation of cell surface markers of immune cell activation, such as CD25, CD80, CD86, and CD154. Methods for measuring cell surface expression of such markers are well known in the art and include FACS analysis.

For measurement of immune response in a cell or population of cells, in one embodiment the cell or population of cells expresses at least one of TLR7, TLR8, or TLR9. The cell can express the TLR naturally, or it can be manipulated to express the TLR though introduction into the cell of a suitable expression vector for the TLR. In one embodiment the cell or population of cells is obtained as peripheral blood mononuclear cells (PBMC). In one embodiment the cell or population of cells is obtained as a cell line expressing the TLR. In one embodiment the cell or population of cells is obtained as a stable transfectant expressing the TLR.

Also for use in measuring an immune response in a cell or population of cells, it may be convenient to introduce into the cell or population of cells a reporter construct that is responsive to intracellular signaling by a TLR. In one embodiment such a reporter is a gene placed under the control of an NF-κB promoter. In one embodiment the gene placed under control of the promoter is luciferase. Under suitable conditions of activation, the reporter construct is expressed and emits a detectable light signal that may be measured quantitatively using a luminometer. Such reporter constructs and other suitable reporter constructs are commercially available.

The invention also contemplates the use of cell-free methods of detecting TLR activation.

The invention in certain aspects relates to compositions and methods for use in therapy. The immunostimulatory compositions of the invention can be used alone or combined with other therapeutic agents. The immunostimulatory composition and other therapeutic agent may be administered simultaneously or sequentially. When the immunostimulatory composition of the invention and the other therapeutic agent are administered simultaneously, they can be administered in the same or separate formulations, but they are administered at the same time. In addition, when the immunostimulatory composition of the invention and the other therapeutic agent are administered simultaneously, they can be administered via the same or separate routes of administration, but they are administered at the same time. The immunostimulatory composition of the invention and another therapeutic agent are administered sequentially when administration of the immunostimulatory composition of the invention is temporally separated from administration of the other therapeutic agent. The separation in time between the administration of these compounds may be a matter of minutes or it may be longer. In one embodiment the immunostimulatory composition of the invention is administered before administration of the other therapeutic agent. In one embodiment the immunostimulatory composition of the invention is administered after administration of the other therapeutic agent. In addition, when the immunostimulatory composition of the invention and the other therapeutic agent are administered sequentially, they can be administered via the same or separate routes of administration. Other therapeutic agents include but are not limited to adjuvants, antigens, vaccines, and medicaments useful for the treatment of infection, cancer, allergy, and asthma.

In one aspect the invention provides a method of vaccinating a subject. The method according to this aspect of the invention includes the step of administering to the subject an antigen and a composition of the invention. In one embodiment the administering the antigen includes administering a nucleic acid encoding the antigen.

A “subject” as used herein refers to a vertebrate animal. In various embodiments the subject is a human, a non-human primate, or other mammal. In certain embodiments the subject is a mouse, rat, guinea pig, rabbit, cat, dog, pig, sheep, goat, cow, or horse.

For use in the method of vaccinating a subject, the composition of the invention in one embodiment includes an antigen. The antigen can be separate from or covalently linked to a polymer of the invention. In one embodiment the antigen is a nucleic acid that encodes the antigen. In another embodiment the composition of the invention does not itself include the antigen. In this embodiment the antigen can be administered to the subject either separately from the composition of the invention, or together with the composition of the invention. Administration that is separate includes separate in time, separate in location or route of administration, or separate both in time and in location or route of administration. When the composition of the invention and the antigen are administered separate in time, the antigen can be administered before or after the composition of the invention. In one embodiment the antigen is administered 48 hours to 4 weeks after administration of the composition of the invention. The method also contemplates the administration of one or more booster doses of antigen alone, composition alone, or antigen and composition, following an initial administration of antigen and composition.

It is also contemplated by the invention that a subject can be prepared for a future encounter with an unknown antigen by administering to the subject a composition of the invention, wherein the composition does not include an antigen. According to this embodiment the immune system of the subject is prepared to mount a more vigorous response to an antigen that is later encountered by the subject, for example through environmental or occupational exposure. Such method can be used, for example, for travellers, medical workers, and soldiers likely to be exposed to microbial agents.

In one aspect the invention provides a method of treating a subject having an immune system deficiency. The method according to this aspect of the invention includes the step of administering to the subject an effective amount of a composition of the invention to treat the subject. An “immune system deficiency” as used herein refers to an abnormally depressed ability of an immune system to mount an immune response to an antigen. In one embodiment an immune system deficiency is a disease or disorder in which the subject's immune system is not functioning in normal capacity or in which it would be useful to boost the subject's immune response, for example to eliminate a tumor or cancer or an infection in the subject. A “subject having an immune deficiency” as used herein refers to a subject in which there is a depressed ability of the subject's immune system to mount an immune response to an antigen. Subjects having an immune deficiency include subjects having an acquired immune deficiency as well as subjects having a congenital immune system deficiency. Subjects having acquired immune deficiency include, without limitation, subjects having a chronic inflammatory condition, subjects having chronic renal insufficiency or renal failure, subjects having infection, subjects having cancer, subjects receiving immunosuppressive drugs, subjects receiving other immunosuppressive treatment, and subjects with malnutrition. In one embodiment the subject has a suppressed CD4+ T-cell population. In one embodiment the subject has an infection with human immunodeficiency virus (HIV) or has acquired immunodeficiency syndrome (AIDS). The method according to this aspect of the invention thus provides a method for boosting an immune response or boosting the ability to mount an immune response in a subject in need of a more vigorous immune response.

The compositions and methods of the invention can be used alone or in conjunction with other agents and methods useful for the treatment of infection. In one aspect the invention provides a method of treating a subject having an infection. The method according to this aspect of the invention includes the step of administering to a subject having an infection an effective amount of the composition of the invention to treat the subject.

In one aspect the invention provides a method of treating a subject having an infection. The method according to this aspect of the invention includes the step of administering to a subject having an infection an effective amount of the composition of the invention and an infection medicament to treat the subject.

In one aspect the invention provides a use of a modified oligoribonucleotide analog of the invention for the preparation of a medicament for treating an infection in a subject.

In one aspect the invention provides a composition useful for the treatment of infection. The composition according to this aspect includes a modified oligoribonucleotide analog of the invention and an infection medicament.

As used herein, the term “treat” as used in reference to a subject having a disease or condition shall mean to prevent, ameliorate, or eliminate at least one sign or symptom of the disease or condition in the subject.

A “subject having an infection” is a subject that has a disorder arising from the invasion of the subject, superficially, locally, or systemically, by an infectious microorganism. The infectious microorganism can be a virus, bacterium, fungus, or parasite, as described above.

Infection medicaments include but are not limited to anti-bacterial agents, anti-viral agents, anti-fungal agents and anti-parasitic agents. Phrases such as “anti-infective agent”, “antibiotic”, “anti-bacterial agent”, “anti-viral agent”, “anti-fungal agent”, “anti-parasitic agent” and “parasiticide” have well-established meanings to those of ordinary skill in the art and are defined in standard medical texts. Briefly, anti-bacterial agents kill or inhibit bacteria, and include antibiotics as well as other synthetic or natural compounds having similar functions. Anti-viral agents can be isolated from natural sources or synthesized and are useful for killing or inhibiting viruses. Anti-fungal agents are used to treat superficial fungal infections as well as opportunistic and primary systemic fungal infections. Anti-parasite agents kill or inhibit parasites. Many antibiotics are low molecular weight molecules which are produced as secondary metabolites by cells, such as microorganisms. In general, antibiotics interfere with one or more functions or structures which are specific for the microorganism and which are not present in host cells.

One of the problems with anti-infective therapies is the side effects occurring in the host that is treated with the anti-infective agent. For instance, many anti-infectious agents can kill or inhibit a broad spectrum of microorganisms and are not specific for a particular type of species. Treatment with these types of anti-infectious agents results in the killing of the normal microbial flora living in the host, as well as the infectious microorganism. The loss of the microbial flora can lead to disease complications and predispose the host to infection by other pathogens, since the microbial flora compete with and function as barriers to infectious pathogens. Other side effects may arise as a result of specific or non-specific effects of these chemical entities on non-microbial cells or tissues of the host.

Another problem with widespread use of anti-infectants is the development of antibiotic-resistant strains of microorganisms. Already, vancomycin-resistant enterococci, penicillin-resistant pneumococci, multi-resistant S. aureus, and multi-resistant tuberculosis strains have developed and are becoming major clinical problems. Widespread use of anti-infectants will likely produce many antibiotic-resistant strains of bacteria. As a result, new anti-infective strategies will be required to combat these microorganisms.

Antibacterial antibiotics which are effective for killing or inhibiting a wide range of bacteria are referred to as broad-spectrum antibiotics. Other types of antibacterial antibiotics are predominantly effective against the bacteria of the class gram-positive or gram-negative. These types of antibiotics are referred to as narrow-spectrum antibiotics. Other antibiotics which are effective against a single organism or disease and not against other types of bacteria, are referred to as limited-spectrum antibiotics.

Anti-bacterial agents are sometimes classified based on their primary mode of action. In general, anti-bacterial agents are cell wall synthesis inhibitors, cell membrane inhibitors, protein synthesis inhibitors, nucleic acid synthesis or functional inhibitors, and competitive inhibitors. Cell wall synthesis inhibitors inhibit a step in the process of cell wall synthesis, and in general in the synthesis of bacterial peptidoglycan. Cell wall synthesis inhibitors include β-lactam antibiotics, natural penicillins, semi-synthetic penicillins, ampicillin, clavulanic acid, cephalolsporins, and bacitracin.

The β-lactams are antibiotics containing a four-membered β-lactam ring which inhibits the last step of peptidoglycan synthesis. β-lactam antibiotics can be synthesized or natural. The β-lactam antibiotics produced by penicillium are the natural penicillins, such as penicillin G or penicillin V. These are produced by fermentation of Penicillium chrysogenum. The natural penicillins have a narrow spectrum of activity and are generally effective against Streptococcus, Gonococcus, and Staphylococcus. Other types of natural penicillins, which are also effective against gram-positive bacteria, include penicillins F, X, K, and O.

Semi-synthetic penicillins are generally modifications of the molecule 6-aminopenicillanic acid produced by a mold. The 6-aminopenicillanic acid can be modified by addition of side chains which produce penicillins having broader spectrums of activity than natural penicillins or various other advantageous properties. Some types of semi-synthetic penicillins have broad spectrums against gram-positive and gram-negative bacteria, but are inactivated by penicillinase. These semi-synthetic penicillins include ampicillin, carbenicillin, oxacillin, azlocillin, mezlocillin, and piperacillin. Other types of semi-synthetic penicillins have narrower activities against gram-positive bacteria, but have developed properties such that they are not inactivated by penicillinase. These include, for instance, methicillin, dicloxacillin, and nafcillin. Some of the broad spectrum semi-synthetic penicillins can be used in combination with β-lactamase inhibitors, such as clavulanic acids and sulbactam. The β-lactamase inhibitors do not have anti-microbial action but they function to inhibit penicillinase, thus protecting the semi-synthetic penicillin from degradation.

Another type of β-lactam antibiotic is the cephalolsporins. They are sensitive to degradation by bacterial β-lactamases, and thus, are not always effective alone. Cephalolsporins, however, are resistant to penicillinase. They are effective against a variety of gram-positive and gram-negative bacteria. Cephalolsporins include, but are not limited to, cephalothin, cephapirin, cephalexin, cefamandole, cefaclor, cefazolin, cefuroxine, cefoxitin, cefotaxime, cefsulodin, cefetamet, cefixime, ceftriaxone, cefoperazone, ceftazidine, and moxalactam.

Bacitracin is another class of antibiotics which inhibit cell wall synthesis, by inhibiting the release of muropeptide subunits or peptidoglycan from the molecule that delivers the subunit to the outside of the membrane. Although bacitracin is effective against gram-positive bacteria, its use is limited in general to topical administration because of its high toxicity.

Carbapenems are another broad-spectrum β-lactam antibiotic, which is capable of inhibiting cell wall synthesis. Examples of carbapenems include, but are not limited to, imipenems. Monobactams are also broad-spectrum β-lactam antibiotics, and include, eurtreonam. An antibiotic produced by Streptomyces, vancomycin, is also effective against gram-positive bacteria by inhibiting cell membrane synthesis.

Another class of anti-bacterial agents is the anti-bacterial agents that are cell membrane inhibitors. These compounds disorganize the structure or inhibit the function of bacterial membranes. One problem with anti-bacterial agents that are cell membrane inhibitors is that they can produce effects in eukaryotic cells as well as bacteria because of the similarities in phospholipids in bacterial and eukaryotic membranes. Thus these compounds are rarely specific enough to permit these compounds to be used systemically and prevent the use of high doses for local administration.

One clinically useful cell membrane inhibitor is Polymyxin. Polymyxins interfere with membrane function by binding to membrane phospholipids. Polymyxin is effective mainly against Gram-negative bacteria and is generally used in severe Pseudomonas infections or Pseudomonas infections that are resistant to less toxic antibiotics. The severe side effects associated with systemic administration of this compound include damage to the kidney and other organs.

Other cell membrane inhibitors include Amphotericin B and Nystatin which are anti-fungal agents used predominantly in the treatment of systemic fungal infections and Candida yeast infections. Imidazoles are another class of antibiotic that is a cell membrane inhibitor. Imidazoles are used as anti-bacterial agents as well as anti-fungal agents, e.g., used for treatment of yeast infections, dermatophytic infections, and systemic fungal infections. Imidazoles include but are not limited to clotrimazole, miconazole, ketoconazole, itraconazole, and fluconazole.

Many anti-bacterial agents are protein synthesis inhibitors. These compounds prevent bacteria from synthesizing structural proteins and enzymes and thus cause inhibition of bacterial cell growth or function or cell death. In general these compounds interfere with the processes of transcription or translation. Anti-bacterial agents that block transcription include but are not limited to Rifampins and Ethambutol. Rifampins, which inhibit the enzyme RNA polymerase, have a broad spectrum activity and are effective against gram-positive and gram-negative bacteria as well as Mycobacterium tuberculosis. Ethambutol is effective against Mycobacterium tuberculosis.

Anti-bacterial agents which block translation interfere with bacterial ribosomes to prevent mRNA from being translated into proteins. In general this class of compounds includes but is not limited to tetracyclines, chloramphenicol, the macrolides (e.g., erythromycin) and the aminoglycosides (e.g., streptomycin).

The aminoglycosides are a class of antibiotics which are produced by the bacterium Streptomyces, such as, for instance streptomycin, kanamycin, tobramycin, amikacin, and gentamicin. Aminoglycosides have been used against a wide variety of bacterial infections caused by Gram-positive and Gram-negative bacteria. Streptomycin has been used extensively as a primary drug in the treatment of tuberculosis. Gentamicin is used against many strains of Gram-positive and Gram-negative bacteria, including Pseudomonas infections, especially in combination with Tobramycin. Kanamycin is used against many Gram-positive bacteria, including penicillin-resistant Staphylococci. One side effect of aminoglycosides that has limited their use clinically is that at dosages which are essential for efficacy, prolonged use has been shown to impair kidney function and cause damage to the auditory nerves leading to deafness.

Another type of translation inhibitor anti-bacterial agent is the tetracyclines. The tetracyclines are a class of antibiotics that are broad-spectrum and are effective against a variety of gram-positive and gram-negative bacteria. Examples of tetracyclines include tetracycline, minocycline, doxycycline, and chlortetracycline. They are important for the treatment of many types of bacteria but are particularly important in the treatment of Lyme disease. As a result of their low toxicity and minimal direct side effects, the tetracyclines have been overused and misused by the medical community, leading to problems. For instance, their overuse has led to widespread development of resistance.

Anti-bacterial agents such as the macrolides bind reversibly to the 50 S ribosomal subunit and inhibit elongation of the protein by peptidyl transferase or prevent the release of uncharged tRNA from the bacterial ribosome or both. These compounds include erythromycin, roxithromycin, clarithromycin, oleandomycin, and azithromycin. Erythromycin is active against most Gram-positive bacteria, Neisseria, Legionella and Haemophilus, but not against the Enterobacteriaceae. Lincomycin and clindamycin, which block peptide bond formation during protein synthesis, are used against gram-positive bacteria.

Another type of translation inhibitor is chloramphenicol. Chloramphenicol binds the 70 S ribosome inhibiting the bacterial enzyme peptidyl transferase thereby preventing the growth of the polypeptide chain during protein synthesis. One serious side effect associated with chloramphenicol is aplastic anemia. Aplastic anemia develops at doses of chloramphenicol which are effective for treating bacteria in a small proportion ( 1/50,000) of patients. Chloramphenicol which was once a highly prescribed antibiotic is now seldom uses as a result of the deaths from anemia. Because of its effectiveness it is still used in life-threatening situations (e.g., typhoid fever).

Some anti-bacterial agents disrupt nucleic acid synthesis or function, e.g., bind to DNA or RNA so that their messages cannot be read. These include but are not limited to quinolones and co-trimoxazole, both synthetic chemicals and rifamycins, a natural or semi-synthetic chemical. The quinolones block bacterial DNA replication by inhibiting the DNA gyrase, the enzyme needed by bacteria to produce their circular DNA. They are broad spectrum and examples include norfloxacin, ciprofloxacin, enoxacin, nalidixic acid and temafloxacin. Nalidixic acid is a bactericidal agent that binds to the DNA gyrase enzyme (topoisomerase) which is essential for DNA replication and allows supercoils to be relaxed and reformed, inhibiting DNA gyrase activity. The main use of nalidixic acid is in treatment of lower urinary tract infections (UTI) because it is effective against several types of Gram-negative bacteria such as E. coli, Enterobacter aerogenes, K. pneumoniae and Proteus species which are common causes of UTI. Co-trimoxazole is a combination of sulfamethoxazole and trimethoprim, which blocks the bacterial synthesis of folic acid needed to make DNA nucleotides. Rifampicin is a derivative of rifamycin that is active against Gram-positive bacteria (including Mycobacterium tuberculosis and meningitis caused by Neisseria meningitidis) and some Gram-negative bacteria. Rifampicin binds to the beta subunit of the polymerase and blocks the addition of the first nucleotide which is necessary to activate the polymerase, thereby blocking mRNA synthesis.

Another class of anti-bacterial agents is compounds that function as competitive inhibitors of bacterial enzymes. The competitive inhibitors are mostly all structurally similar to a bacterial growth factor and compete for binding but do not perform the metabolic function in the cell. These compounds include sulfonamides and chemically modified forms of sulfanilamide which have even higher and broader antibacterial activity. The sulfonamides (e.g., gantrisin and trimethoprim) are useful for the treatment of Streptococcus pneumoniae, beta-hemolytic streptococci and E. coli, and have been used in the treatment of uncomplicated UTI caused by E. coli, and in the treatment of meningococcal meningitis.

Anti-viral agents are compounds which prevent infection of cells by viruses or replication of the virus within the cell. There are many fewer antiviral drugs than antibacterial drugs because the process of viral replication is so closely related to DNA replication within the host cell, that non-specific antiviral agents would often be toxic to the host. There are several stages within the process of viral infection which can be blocked or inhibited by antiviral agents. These stages include, attachment of the virus to the host cell (immunoglobulin or binding peptides), uncoating of the virus (e.g. amantadine), synthesis or translation of viral mRNA (e.g. interferon), replication of viral RNA or DNA (e.g. nucleoside analogues), maturation of new virus proteins (e.g. protease inhibitors), and budding and release of the virus.

Another category of anti-viral agents are nucleoside analogues. Nucleoside analogues are synthetic compounds which are similar to nucleosides, but which have an incomplete or abnormal deoxyribose or ribose group. Once the nucleoside analogues are in the cell, they are phosphorylated, producing the triphosphate form which competes with normal nucleotides for incorporation into the viral DNA or RNA. Once the triphosphate form of the nucleoside analogue is incorporated into the growing nucleic acid chain, it causes irreversible association with the viral polymerase and thus chain termination. Nucleoside analogues include, but are not limited to, acyclovir (used for the treatment of herpes simplex virus and varicella-zoster virus), gancyclovir (useful for the treatment of cytomegalovirus), idoxuridine, ribavirin (useful for the treatment of respiratory syncitial virus), dideoxyinosine, dideoxycytidine, and zidovudine (azidothymidine).

Another class of anti-viral agents includes cytokines such as interferons. The interferons are cytokines which are secreted by virus-infected cells as well as immune cells. The interferons function by binding to specific receptors on cells adjacent to the infected cells, causing the change in the cell which protects it from infection by the virus. α and β-interferon also induce the expression of Class I and Class II MHC molecules on the surface of infected cells, resulting in increased antigen presentation for host immune cell recognition. α and β-interferons are available as recombinant forms and have been used for the treatment of chronic hepatitis B and C infection. At the dosages which are effective for anti-viral therapy, interferons have severe side effects such as fever, malaise and weight loss.

Immunoglobulin therapy is used for the prevention of viral infection. Immunoglobulin therapy for viral infections is different from bacterial infections, because rather than being antigen-specific, the immunoglobulin therapy functions by binding to extracellular virions and preventing them from attaching to and entering cells which are susceptible to the viral infection. The therapy is useful for the prevention of viral infection for the period of time that the antibodies are present in the host. In general there are two types of immunoglobulin therapies, normal immune globulin therapy and hyper-immune globulin therapy. Normal immune globulin therapy utilizes a antibody product which is prepared from the serum of normal blood donors and pooled. This pooled product contains low titers of antibody to a wide range of human viruses, such as hepatitis A, parvovirus, enterovirus (especially in neonates). Hyper-immune globulin therapy utilizes antibodies which are prepared from the serum of individuals who have high titers of an antibody to a particular virus. Those antibodies are then used against a specific virus. Examples of hyper-immune globulins include zoster immune globulin (useful for the prevention of varicella in immunocompromised children and neonates), human rabies immune globulin (useful in the post-exposure prophylaxis of a subject bitten by a rabid animal), hepatitis B immune globulin (useful in the prevention of hepatitis B virus, especially in a subject exposed to the virus), and RSV immune globulin (useful in the treatment of respiratory syncitial virus infections).

Anti-fungal agents are useful for the treatment and prevention of infective fungi. Anti-fungal agents are sometimes classified by their mechanism of action. Some anti-fungal agents function as cell wall inhibitors by inhibiting glucose synthase. These include, but are not limited to, basiungin/ECB. Other anti-fungal agents function by destabilizing membrane integrity. These include, but are not limited to, imidazoles, such as clotrimazole, sertaconzole, fluconazole, itraconazole, ketoconazole, miconazole, and voriconacole, as well as FK 463, amphotericin B, BAY 38-9502, MK 991, pradimicin, UK 292, butenafine, and terbinafine. Other anti-fungal agents function by breaking down chitin (e.g., chitinase) or immunosuppression (501 cream).

Parasiticides are agents that kill parasites directly. Such compounds are known in the art and are generally commercially available. Examples of parasiticides useful for human administration include but are not limited to albendazole, amphotericin B, benznidazole, bithionol, chloroquine HCl, chloroquine phosphate, clindamycin, dehydroemetine, diethylcarbamazine, diloxanide furoate, eflornithine, furazolidaone, glucocorticoids, halofantrine, iodoquinol, ivermectin, mebendazole, mefloquine, meglumine antimoniate, melarsoprol, metrifonate, metronidazole, niclosamide, nifurtimox, oxamniquine, paromomycin, pentamidine isethionate, piperazine, praziquantel, primaquine phosphate, proguanil, pyrantel pamoate, pyrimethanmine-sulfonamides, pyrimethanmine-sulfadoxine, quinacrine HCl, quinine sulfate, quinidine gluconate, spiramycin, stibogluconate sodium (sodium antimony gluconate), suramin, tetracycline, doxycycline, thiabendazole, tinidazole, trimethroprim-sulfamethoxazole, and tryparsamide.

The compositions and methods of the invention can be used alone or in conjunction with other agents and methods useful for the treatment of cancer. In one aspect the invention provides a method of treating a subject having a cancer. The method according to this aspect of the invention includes the step of administering to a subject having a cancer an effective amount of a composition of the invention to treat the subject.

In one aspect the invention provides a method of treating a subject having a cancer. The method according to this aspect of the invention includes the step of administering to a subject having a cancer an effective amount of the composition of the invention and an anti-cancer therapy to treat the subject.

In one aspect the invention provides a use of a modified oligoribonucleotide analog of the invention for the preparation of a medicament for treating cancer in a subject.

In one aspect the invention provides a composition useful for the treatment of cancer. The composition according to this aspect includes a modified oligoribonucleotide analog of the invention and a cancer medicament.

A subject having a cancer is a subject that has detectable cancerous cells. The cancer may be a malignant or non-malignant cancer. “Cancer” as used herein refers to an uncontrolled growth of cells which interferes with the normal functioning of the bodily organs and systems. Cancers which migrate from their original location and seed vital organs can eventually lead to the death of the subject through the functional deterioration of the affected organs. Hemopoietic cancers, such as leukemia, are able to outcompete the normal hemopoietic compartments in a subject, thereby leading to hemopoietic failure (in the form of anemia, thrombocytopenia and neutropenia) ultimately causing death.

A metastasis is a region of cancer cells, distinct from the primary tumor location, resulting from the dissemination of cancer cells from the primary tumor to other parts of the body. At the time of diagnosis of the primary tumor mass, the subject may be monitored for the presence of metastases. Metastases are most often detected through the sole or combined use of magnetic resonance imaging (MRI) scans, computed tomography (CT) scans, blood and platelet counts, liver function studies, chest X-rays and bone scans in addition to the monitoring of specific symptoms.

Cancers include, but are not limited to, basal cell carcinoma, biliary tract cancer; bladder cancer; bone cancer; brain and central nervous system (CNS) cancer; breast cancer; cervical cancer; choriocarcinoma; colon and rectum cancer; connective tissue cancer; cancer of the digestive system; endometrial cancer; esophageal cancer; eye cancer; cancer of the head and neck; intra-epithelial neoplasm; kidney cancer; larynx cancer; leukemia; liver cancer; lung cancer (e.g. small cell and non-small cell); lymphoma including Hodgkin's and Non-Hodgkin's lymphoma; melanoma; myeloma; neuroblastoma; oral cavity cancer (e.g., lip, tongue, mouth, and pharynx); ovarian cancer; pancreatic cancer; prostate cancer; retinoblastoma; rhabdomyosarcoma; rectal cancer; cancer of the respiratory system; sarcoma; skin cancer; stomach cancer; testicular cancer; thyroid cancer; uterine cancer; cancer of the urinary system, as well as other carcinomas, adenocarcinomas, and sarcomas.

The immunostimulatory composition of the invention may also be administered in conjunction with an anti-cancer therapy. Anti-cancer therapies include cancer medicaments, radiation, and surgical procedures. As used herein, a “cancer medicament” refers to an agent which is administered to a subject for the purpose of treating a cancer. As used herein, “treating cancer” includes preventing the development of a cancer, reducing the symptoms of cancer, and/or inhibiting the growth of an established cancer. In other aspects, the cancer medicament is administered to a subject at risk of developing a cancer for the purpose of reducing the risk of developing the cancer. Various types of medicaments for the treatment of cancer are described herein. For the purpose of this specification, cancer medicaments are classified as chemotherapeutic agents, immunotherapeutic agents, cancer vaccines, hormone therapy, and biological response modifiers.

The chemotherapeutic agent may be selected from the group consisting of methotrexate, vincristine, adriamycin, cisplatin, non-sugar containing chloroethylnitrosoureas, 5-fluorouracil, mitomycin C, bleomycin, doxorubicin, dacarbazine, taxol, fragyline, Meglamine GLA, valrubicin, carmustaine and poliferposan, MMI270, BAY 12-9566, RAS famesyl transferase inhibitor, famesyl transferase inhibitor, MMP, MTA/LY231514, LY264618/Lometexol, Glamolec, CI-994, TNP-470, Hycamtin/Topotecan, PKC412, Valspodar/PSC833, Novantrone/Mitroxantrone, Metaret/Suramin, Batimastat, E7070, BCH-4556, CS-682, 9-AC, AG3340, AG3433, Incel/VX-710, VX-853, ZD0101, ISI641, ODN 698, TA 2516/Marmistat, BB2516/Marmistat, CDP 845, D2163, PD183805, DX8951f, Lemonal DP 2202, FK 317, Picibanil/OK-432, AD 32Nalrubicin, Metastron/strontium derivative, Temodal/Temozolomide,Evacet/liposomal doxorubicin, Yewtaxan/Paclitaxel, Taxol/Paclitaxel, Xeload/Capecitabine, Furtulon/Doxifluridine, Cyclopax/oral paclitaxel, Oral Taxoid, SPU-077/Cisplatin, HMR 1275/Flavopiridol, CP-358 (774)/EGFR, CP-609 (754)/RAS oncogene inhibitor, BMS-182751/oral platinum, UFT(Tegafur/Uracil), Ergamisol/Levamisole, Eniluracil/776C85/5FU enhancer, Campto/Levamisole, Camptosar/Irinotecan, Tumodex/Ralitrexed, Leustatin/Cladribine, Paxex/Paclitaxel, Doxil/liposomal doxorubicin, Caelyx/liposomal doxorubicin, Fludara/Fludarabine, Pharmarubicin/Epirubicin, DepoCyt, ZD1839, LU 79553/Bis-Naphtalimide, LU 103793/Dolastain, Caetyx/liposomal doxorubicin, Gemzar/Gemcitabine, ZD 0473/Anormed, YM 116, Iodine seeds, CDK4 and CDK2 inhibitors, PARP inhibitors, D4809/Dexifosamide, Ifes/Mesnex/Ifosamide, Vumon/Teniposide, Paraplatin/Carboplatin, Plantinol/cisplatin, Vepeside/Etoposide, ZD 9331, Taxotere/Docetaxel, prodrug of guanine arabinoside, Taxane Analog, nitrosoureas, alkylating agents such as melphelan and cyclophosphamide, Aminoglutethimide, Asparaginase, Busulfan, Carboplatin, Chlorombucil, Cytarabine HCl, Dactinomycin, Daunorubicin HCl, Estramustine phosphate sodium, Etoposide (VP 16-213), Floxuridine, Fluorouracil (5-FU), Flutamide, Hydroxyurea (hydroxycarbamide), Ifosfamide, Interferon Alfa-2a, Alfa-2b, Leuprolide acetate (LHRH-releasing factor analogue), Lomustine (CCNU), Mechlorethamine HCl (nitrogen mustard), Mercaptopurine, Mesna, Mitotane (o.p′-DDD), Mitoxantrone HCl, Octreotide, Plicamycin, Procarbazine HCl, Streptozocin, Tamoxifen citrate, Thioguanine, Thiotepa, Vinblastine sulfate, Amsacrine (m-AMSA), Azacitidine, Erthropoietin, Hexamethylmelamine (HMM), Interleukin 2, Mitoguazone (methyl-GAG; methyl glyoxal bis-guanylhydrazone; MGBG), Pentostatin (2′deoxycoformycin), Semustine (methyl-CCNU), Teniposide (VM-26) and Vindesine sulfate, but it is not so limited.

The immunotherapeutic agent may be selected from the group consisting of 3622W94, 4B5, ANA Ab, anti-FLK-2, anti-VEGF, ATRAGEN, AVASTIN (bevacizumab; Genentech), BABS, BEC2, BEXXAR (tositumomab; GlaxoSmithKline), C225, CAMPATH (alemtuzumab; Genzyme Corp.), CEACIDE, CMA 676, EMD-72000, ERBITUX (cetuximab; ImClone Systems, Inc.), Gliomab-H, GNI-250, HERCEPTIN (trastuzumab; Genentech), IDEC-Y2B8, ImmuRAIT-CEA, ior c5, ior egf.r3, ior t6, LDP-03, LymphoCide, MDX-11, MDX-22, MDX-210, MDX-220, MDX-260, MDX-447, MELIMMUNE-1, MELIMMUNE-2, Monopharm-C, NovoMAb-G2, Oncolym, OV103, Ovarex, Panorex, Pretarget, Quadramet, Ributaxin, RITUXAN (rituximab; Genentech), SMART 1D10 Ab, SMART ABL 364 Ab, SMART M195, TNT, and ZENAPAX (daclizumab; Roche), but it is not so limited.

The cancer vaccine may be selected from the group consisting of EGF, Anti-idiotypic cancer vaccines, Gp75 antigen, GMK melanoma vaccine, MGV ganglioside conjugate vaccine, Her2/neu, Ovarex, M-Vax, O-Vax, L-Vax, STn-KHL theratope, BLP25 (MUC-1), liposomal idiotypic vaccine, Melacine, peptide antigen vaccines, toxin/antigen vaccines, MVA-based vaccine, PACIS, BCG vacine, TA-HPV, TA-CIN, DISC-virus and ImmuCyst/TheraCys, but it is not so limited.

The compositions and methods of the invention can be used alone or in conjunction with other agents and methods useful for the treatment of allergy. In one aspect the invention provides a method of treating a subject having an allergic condition. The method according to this aspect of the invention includes the step of administering to a subject having an allergic condition an effective amount of a composition of the invention to treat the subject.

In one aspect the invention provides a method of treating a subject having an allergic condition. The method according to this aspect of the invention includes the step of administering to a subject having an allergic condition an effective amount of the composition of the invention and an anti-allergy therapy to treat the subject.

In one aspect the invention provides a use of a modified oligoribonucleotide analog of the invention for the preparation of a medicament for treating an allergic condition in a subject.

In one aspect the invention provides a composition useful for the treatment of an allergic condition. The composition according to this aspect includes a modified oligoribonucleotide analog of the invention and an allergy medicament.

A “subject having an allergic condition” shall refer to a subject that is currently experiencing or has previously experienced an allergic reaction in response to an allergen.

An “allergic condition” or “allergy” refers to acquired hypersensitivity to a substance (allergen). Allergic conditions include but are not limited to eczema, allergic rhinitis or coryza, hay fever, allergic conjunctivitis, bronchial asthma, urticaria (hives) and food allergies, other atopic conditions including atopic dermatitis; anaphylaxis; drug allergy; and angioedema.

Allergy is typically an episodic condition associated with the production of antibodies from a particular class of immunoglobulin, IgE, against allergens. The development of an IgE-mediated response to common aeroallergens is also a factor which indicates predisposition towards the development of asthma. If an allergen encounters a specific IgE which is bound to an IgE Fc receptor (FcsR) on the surface of a basophil (circulating in the blood) or mast cell (dispersed throughout solid tissue), the cell becomes activated, resulting in the production and release of mediators such as histamine, serotonin, and lipid mediators.

An allergic reaction occurs when tissue-sensitizing immunoglobulin of the IgE type reacts with foreign allergen. The IgE antibody is bound to mast cells and/or basophils, and these specialized cells release chemical mediators (vasoactive amines) of the allergic reaction when stimulated to do so by allergens bridging the ends of the antibody molecule. Histamine, platelet activating factor, arachidonic acid metabolites, and serotonin are among the best known mediators of allergic reactions in man. Histamine and the other vasoactive amines are normally stored in mast cells and basophil leukocytes. The mast cells are dispersed throughout animal tissue and the basophils circulate within the vascular system. These cells manufacture and store histamine within the cell unless the specialized sequence of events involving IgE binding occurs to trigger its release.

Symptoms of an allergic reaction vary, depending on the location within the body where the IgE reacts with the antigen. If the reaction occurs along the respiratory epithelium, the symptoms generally are sneezing, coughing and asthmatic reactions. If the interaction occurs in the digestive tract, as in the case of food allergies, abdominal pain and diarrhea are common. Systemic allergic reactions, for example following a bee sting or administration of penicillin to an allergic subject, can be severe and often life-threatening.

Allergy is associated with a Th2-type of immune response, which is characterized at least in part by Th2 cytokines IL-4 and IL-5, as well as antibody isotype switching to IgE. Th1 and Th2 immune responses are mutually counter-regulatory, so that skewing of the immune response toward a Th1-type of immune response can prevent or ameliorate a Th2-type of immune response, including allergy. The modified oligoribonucleotide analogs of the invention are therefore useful by themselves to treat a subject having an allergic condition because the analogs can skew the immune response toward a Th1-type of immune response. Alternatively or in addition, the modified oligoribonucleotide analogs of the invention can be used in combination with an allergen to treat a subject having an allergic condition.

The immunostimulatory composition of the invention may also be administered in conjunction with an anti-allergy therapy. Conventional methods for treating or preventing allergy have involved the use of anti-histamines or desensitization therapies. Some evolving therapies for treating or preventing allergy include the use of neutralizing anti-IgE antibodies. Anti-histamines and other drugs which block the effects of chemical mediators of the allergic reaction help to regulate the severity of the allergic symptoms but do not prevent the allergic reaction and have no effect on subsequent allergic responses. Desensitization therapies are performed by giving small doses of an allergen, usually by injection under the skin, in order to induce an IgG-type response against the allergen. The presence of IgG antibody helps to neutralize the production of mediators resulting from the induction of IgE antibodies, it is believed. Initially, the subject is treated with a very low dose of the allergen to avoid inducing a severe reaction and the dose is slowly increased. This type of therapy is dangerous because the subject is actually administered the compounds which cause the allergic response and severe allergic reactions can result.

Allergy medicaments include, but are not limited to, anti-histamines, corticosteroids, and prostaglandin inducers. Anti-histamines are compounds which counteract histamine released by mast cells or basophils. These compounds are well known in the art and commonly used for the treatment of allergy. Anti-histamines include, but are not limited to, acrivastine, astemizole, azatadine, azelastine, betatastine, brompheniramine, buclizine, cetirizine, cetirizine analogues, chlorpheniramine, clemastine, CS 560, cyproheptadine, desloratadine, dexchlorpheniramine, ebastine, epinastine, fexofenadine, HSR 609, hydroxyzine, levocabastine, loratidine, methscopolamine, mizolastine, norastemizole, phenindamine, promethazine, pyrilamine, terfenadine, and tranilast.

Corticosteroids include, but are not limited to, methylprednisolone, prednisolone, prednisone, beclomethasone, budesonide, dexamethasone, flunisolide, fluticasone propionate, and triamcinolone. Although dexamethasone is a corticosteroid having anti-inflammatory action, it is not regularly used for the treatment of allergy or asthma in an inhaled form because it is highly absorbed and it has long-term suppressive side effects at an effective dose. Dexamethasone, however, can be used according to the invention for treating allergy or asthma because when administered in combination with a composition of the invention it can be administered at a low dose to reduce the side effects. Some of the side effects associated with corticosteroid use include cough, dysphonia, oral thrush (candidiasis), and in higher doses, systemic effects, such as adrenal suppression, glucose intolerance, osteoporosis, aseptic necrosis of bone, cataract formation, growth suppression, hypertension, muscle weakness, skin thinning, and easy bruising. Barnes & Peterson (1993) Am Rev Respir Dis 148:S1-S26; and Kamada A K et al. (1996) Am J Respir Crit Care Med 153:1739-48.

The compositions and methods of the invention can be used alone or in conjunction with other agents and methods useful for the treatment of asthma. In one aspect the invention provides a method of treating a subject having asthma. The method according to this aspect of the invention includes the step of administering to a subject having asthma an effective amount of a composition of the invention to treat the subject.

In one aspect the invention provides a method of treating a subject having asthma. The method according to this aspect of the invention includes the step of administering to a subject having asthma an effective amount of the composition of the invention and an anti-asthma therapy to treat the subject.

In one aspect the invention provides a use of a modified oligoribonucleotide analog of the invention for the preparation of a medicament for treating asthma in a subject.

In one aspect the invention provides a composition useful for the treatment of asthma. The composition according to this aspect includes a modified ribonucleotide analog of the invention and an asthma medicament.

“Asthma” as used herein refers to a disorder of the respiratory system characterized by inflammation and narrowing of the airways, and increased reactivity of the airways to inhaled agents. Asthma is frequently, although not exclusively, associated with an atopic or allergic condition. Symptoms of asthma include recurrent episodes of wheezing, breathlessness, chest tightness, and coughing, resulting from airflow obstruction. Airway inflammation associated with asthma can be detected through observation of a number of physiological changes, such as, denudation of airway epithelium, collagen deposition beneath basement membrane, edema, mast cell activation, inflammatory cell infiltration, including neutrophils, eosinophils, and lymphocytes. As a result of the airway inflammation, asthma patients often experience airway hyper-responsiveness, airflow limitation, respiratory symptoms, and disease chronicity. Airflow limitations include acute bronchoconstriction, airway edema, mucous plug formation, and airway remodeling, features which often lead to bronchial obstruction. In some cases of asthma, sub-basement membrane fibrosis may occur, leading to persistent abnormalities in lung function.

Research over the past several years has revealed that asthma likely results from complex interactions among inflammatory cells, mediators, and other cells and tissues resident in the airways. Mast cells, eosinophils, epithelial cells, macrophage, and activated T cells all play an important role in the inflammatory process associated with asthma. Djukanovic R et al. (1990) Am Rev Respir Dis 142:434-457. It is believed that these cells can influence airway function through secretion of preformed and newly synthesized mediators which can act directly or indirectly on the local tissue. It has also been recognized that subpopulations of T lymphocytes (Th2) play an important role in regulating allergic inflammation in the airway by releasing selective cytokines and establishing disease chronicity. Robinson D S et al. (1992) N Engl J Med 326:298-304.

Asthma is a complex disorder which arises at different stages in development and can be classified based on the degree of symptoms as acute, subacute, or chronic. An acute inflammatory response is associated with an early recruitment of cells into the airway. The subacute inflammatory response involves the recruitment of cells as well as the activation of resident cells causing a more persistent pattern of inflammation. Chronic inflammatory response is characterized by a persistent level of cell damage and an ongoing repair process, which may result in permanent abnormalities in the airway.

A “subject having asthma” is a subject that has a disorder of the respiratory system characterized by inflammation and narrowing of the airways and increased reactivity of the airways to inhaled agents. Factors associated with initiation of asthma include, but are not limited to, allergens, cold temperature, exercise, viral infections, and SO₂.

As mentioned above, asthma may be associated with a Th2-type of immune response, which is characterized at least in part by Th2 cytokines IL-4 and IL-5, as well as antibody isotype switching to IgE. Th1 and Th2 immune responses are mutually counter-regulatory, so that skewing of the immune response toward a Th1-type of immune response can prevent or ameliorate a Th2-type of immune response, including allergy. The modified oligoribonucleotide analogs of the invention are therefore useful by themselves to treat a subject having asthma because the analogs can skew the immune response toward a Th1-type of immune response. Alternatively or in addition, the modified oligoribonucleotide analogs of the invention can be used in combination with an allergen to treat a subject having asthma.

The immunostimulatory composition of the invention may also be administered in conjunction with an asthma therapy. Conventional methods for treating or preventing asthma have involved the use of anti-allergy therapies (described above) and a number of other agents, including inhaled agents.

Medications for the treatment of asthma are generally separated into two categories, quick-relief medications and long-term control medications. Asthma patients take the long-term control medications on a daily basis to achieve and maintain control of persistent asthma. Long-term control medications include anti-inflammatory agents such as corticosteroids, chromolyn sodium and nedocromil; long-acting bronchodilators, such as long-acting β₂-agonists and methylxanthines; and leukotriene modifiers. The quick-relief medications include short-acting agonists, anti-cholinergics, and systemic corticosteroids. There are many side effects associated with each of these drugs and none of the drugs alone or in combination is capable of preventing or completely treating asthma.

Asthma medicaments include, but are not limited, PDE-4 inhibitors, bronchodilator/beta-2 agonists, K+ channel openers, VLA-4 antagonists, neurokin antagonists, thromboxane A2 (TXA2) synthesis inhibitors, xanthines, arachidonic acid antagonists, 5 lipoxygenase inhibitors, TXA2 receptor antagonists, TXA2 antagonists, inhibitor of 5-lipox activation proteins, and protease inhibitors.

Bronchodilator/β₂ agonists are a class of compounds which cause bronchodilation or smooth muscle relaxation. Bronchodilator/β₂ agonists include, but are not limited to, salmeterol, salbutamol, albuterol, terbutaline, D2522/formoterol, fenoterol, bitolterol, pirbuerol methylxanthines and orciprenaline. Long-acting β2 agonists and bronchodilators are compounds which are used for long-term prevention of symptoms in addition to the anti-inflammatory therapies. Long-acting β₂ agonists include, but are not limited to, salmeterol and albuterol. These compounds are usually used in combination with corticosteroids and generally are not used without any inflammatory therapy. They have been associated with side effects such as tachycardia, skeletal muscle tremor, hypokalemia, and prolongation of QTc interval in overdose.

Methylxanthines, including for instance theophylline, have been used for long-term control and prevention of symptoms. These compounds cause bronchodilation resulting from phosphodiesterase inhibition and likely adenosine antagonism. Dose-related acute toxicities are a particular problem with these types of compounds. As a result, routine serum concentration must be monitored in order to account for the toxicity and narrow therapeutic range arising from individual differences in metabolic clearance. Side effects include tachycardia, tachyarrhythmias, nausea and vomiting, central nervous system stimulation, headache, seizures, hematemesis, hyperglycemia and hypokalemia. Short-acting β₂ agonists include, but are not limited to, albuterol, bitolterol, pirbuterol, and terbutaline. Some of the adverse effects associated with the administration of short-acting β₂ agonists include tachycardia, skeletal muscle tremor, hypokalemia, increased lactic acid, headache, and hyperglycemia.

Chromolyn sodium and nedocromil are used as long-term control medications for preventing primarily asthma symptoms arising from exercise or allergic symptoms arising from allergens. These compounds are believed to block early and late reactions to allergens by interfering with chloride channel function. They also stabilize mast cell membranes and inhibit activation and release of mediators from inosineophils and epithelial cells. A four to six week period of administration is generally required to achieve a maximum benefit.

Anticholinergics are generally used for the relief of acute bronchospasm. These compounds are believed to function by competitive inhibition of muscarinic cholinergic receptors. Anticholinergics include, but are not limited to, ipratropium bromide. These compounds reverse only cholinerigically-mediated bronchospasm and do not modify any reaction to antigen. Side effects include drying of the mouth and respiratory secretions, increased wheezing in some individuals, and blurred vision if sprayed in the eyes.

The modified oligoribonucleotide analogs of the invention may also be useful for treating airway remodeling. Airway remodeling results from smooth muscle cell proliferation and/or submucosal thickening in the airways, and ultimately causes narrowing of the airways leading to restricted airflow. The modified oligoribonucleotide analogs of the invention may prevent further remodeling and possibly even reduce tissue build-up resulting from the remodeling process.

The modified oligoribonucleotide analogs of the invention are also useful for improving survival, differentiation, activation and maturation of dendritic cells. The modified oligoribonucleotide analogs have the unique capability to promote cell survival, differentiation, activation and maturation of dendritic cells.

Modified oligoribonucleotide analogs of the invention also increase natural killer cell lytic activity and antibody-dependent cellular cytotoxicity (ADCC). ADCC can be performed using a modified oligoribonucleotide analog in combination with an antibody specific for a cellular target, such as a cancer cell. When the modified oligoribonucleotide analog is administered to a subject in conjunction with the antibody, the subject's immune system is induced to kill the tumor cell. The antibodies useful in the ADCC procedure include antibodies which interact with a cell in the body. Many such antibodies specific for cellular targets have been described in the art and many are commercially available. In one embodiment the antibody is an IgG antibody.

In certain aspects the invention provides a method for enhancing epitope spreading. “Epitope spreading” as used herein refers to the diversification of epitope specificity from an initial focused, dominant epitope-specific immune response, directed against a self or foreign protein, to subdominant and/or cryptic epitopes on that protein (intramolecular spreading) or other proteins (intermolecular spreading). Epitope spreading results in multiple epitope-specific immune responses.

The immune response consists of an initial magnification phase, which can either be deleterious, as in autoimmune disease, or beneficial, as in vaccinations, and a later down-regulatory phase to return the immune system to homeostasis and generate memory. Epitope spreading may be an important component of both phases. The enhancement of epitope spreading in the setting of a tumor allows the subject's immune system to determine additional target epitopes, not initially recognized by the immune system in response to an original therapeutic protocol, while reducing the possibility of escape variants in the tumor population and thus affect progression of disease.

It has been discovered that oligoribonucleotides of the invention are useful for promoting epitope spreading in therapeutically beneficial indications such as cancer, viral and bacterial infections, and allergy. The method in one embodiment includes the steps of administering a vaccine that includes an antigen and an adjuvant to a subject and subsequently administering to the subject at least two doses of modified oligoribonucleotide analog of the invention in an amount effective to induce multiple epitope-specific immune responses. The method in one embodiment includes the steps of administering a vaccine that includes a tumor antigen and an adjuvant to a subject and subsequently administering to the subject at least two doses of modified oligoribonucleotide analog of the invention in an amount effective to induce multiple epitope-specific immune responses. The method in one embodiment involves applying a therapeutic protocol which results in immune system antigen exposure in a subject, followed by at least two administrations of a modified oligoribonucleotide analog of the invention, to induce multiple epitope-specific immune responses, i.e., to promote epitope spreading. In various embodiments the therapeutic protocol is surgery, radiation, chemotherapy, other cancer medicaments, a vaccine, or a cancer vaccine.

The therapeutic protocol may be implemented in conjunction with an immunostimulant, in addition to the subsequent immunostimulant therapy. For instance, when the therapeutic protocol is a vaccine, it may be administered in conjunction with an adjuvant. The combination of the vaccine and the adjuvant may be a mixture or separate administrations, i.e., injections (i.e., same drainage field). Administration is not necessarily simultaneous. If non-simultaneous injection is used, the timing may involve pre-injection of the adjuvant followed by the vaccine formulation.

After the therapeutic protocol is implemented, immunostimulant monotherapy begins. The optimized frequency, duration, and site of administration will depend on the target and other factors, but may for example be a monthly to bi-monthly administration for a period of six months to two years. Alternatively the administration may be on a daily, weekly, or biweekly basis, or the administration may be multiple times during a day, week or month. In some instances, the duration of administration may depend on the length of therapy, e.g., it may end after one week, one month, after one year, or after multiple years. In other instances the monotherapy may be continuous as with an intravenous drip. The immunostimulant may be administered to a drainage field common to the target.

The invention also provides a method for identifying a candidate inhibitor of signaling mediated by TLR7 or TLR8. The method employs a TLR chosen from TLR7 and TLR8, a modified oligoribonucleotide analog of the invention, and a test agent. The selected TLR is contacted with a test agent and a modified oligoribonucleotide analog (TLR ligand), and a test signal mediated by the TLR is measured. The test signal is compared to a control signal, the control signal corresponding to a signal mediated by the TLR as measured in presence of the modified oligoribonucleotide analog but in absence of the test compound. The test agent is identified as a candidate inhibitor of TLR signaling when the control signal exceeds the test signal. Such method is adaptable to automated, high throughput screening of test agents. Examples of such high throughput screening methods are described in U.S. Pat. Nos. 6,103,479; 6,051,380; 6,051,373; 5,998,152; 5,876,946; 5,708,158; 5,443,791; 5,429,921; and 5,143,854.

In one embodiment a “TLR-mediated signal” refers to an ability of a TLR polypeptide to activate the Toll/IL-1R (TIR) signaling pathway, also referred to herein as the TLR signal transduction pathway. Changes in TLR activity can be measured by assays designed to measure expression of genes under control of κB-sensitive promoters and enhancers. Such genes can be naturally occurring genes or they can be genes artificially introduced into a cell. Naturally occurring reporter genes include the genes encoding IL-1β, IL-6, IL-8, the p40 subunit of interleukin 12 (IL-12 p40), and the costimulatory molecules CD80 and CD86. Other genes can be placed under the control of such regulatory elements and thus serve to report the level of TLR signaling.

In another embodiment, a TLR-mediated signal refers to binding or physical interaction between the TLR and the modified oligoribonucleotide analog. This embodiment may be or particular use in connection with performance of the method using a cell-free system. For example, the signal may relate to an interaction as measured using surface plasmon resonance.

The test assay mixture includes a test agent. Typically, a plurality of test assay mixtures are run in parallel with different agent concentrations to obtain a different response to the various concentrations. Typically, one of these concentrations serves as a negative control, i.e., at zero concentration of test agent or at a concentration of agent below the limits of assay detection. Test agents may encompass numerous chemical classes, although typically they are organic compounds. In some embodiments, the test agents are small organic compounds, i.e., organic compounds having a molecular weight of more than 50 yet less than about 2500 Daltons. In addition to small organic compounds, test agents can be biomolecules such as nucleic acids, peptides, saccharides, fatty acids, sterols, isoprenoids, purines, pyrimidines, derivatives or structural analogs of the above, or combinations thereof and the like. In some embodiments the test agent is an RNA with a molecular weight of less than about 2500 Daltons. Polymeric test agents can have higher molecular weights, e.g., oligonucleotides in the range of about 2500 to about 12,500. Where the test agent is a nucleic acid, it typically is a DNA or RNA molecule, although modified nucleic acids having non-natural bonds or subunits are also contemplated.

Test agents may be obtained from a wide variety of sources, including libraries of natural, synthetic, or semisynthetic compounds, or any combination thereof. For example, numerous methods are available for random and directed synthesis of a wide variety of organic compounds and biomolecules, including expression of randomized oligonucleotides, synthetic organic combinatorial libraries, phage display libraries of random peptides, and the like. Alternatively, libraries of natural compounds in the form of bacterial, fungal, plant and animal extracts are available or readily produced. Additionally, natural and synthetically produced libraries and compounds can be readily modified-through conventional chemical, physical, and biochemical means. Further, known pharmacological agents may be subjected to directed or random chemical modifications such as acylation, alkylation, esterification, amidification, etc., to produce structural analogs of the test agents.

A variety of other reagents also can be included in the mixture. These include reagents such as salts, buffers, neutral proteins (e.g., albumin), detergents, etc., which may be used to facilitate optimal protein-protein and/or protein-nucleic acid binding. Such a reagent may also reduce non-specific or background interactions of the reaction components. Other reagents that improve the efficiency of the assay such as protease inhibitors, nuclease inhibitors, antimicrobial agents, and the like may also be used.

The method can be performed as a cell-based assay or as a cell-free assay. For cell-based assays, in one embodiment the TLR is expressed naturally by a cell. In another embodiment the TLR is expressed by a cell that has been manipulated to do so, for example a cell that, but for inclusion of an expression vector for the TLR, normally does not express the TLR (see above). In one embodiment the cell is an HEK-293 cell stably transfected with an expression vector for a TLR7 or a TLR8. A cell in a cell-based assay may optionally include a reporter construct that is responsive to signaling mediated by the TLR (see above).

The order of addition of components, incubation temperature, time of incubation, and other parameters of the assay may be readily determined. Such experimentation merely involves optimization of the assay parameters, not the fundamental composition of the assay. Incubation temperatures typically are between 4° C. and 40° C., more typically about 37° C. Incubation times preferably are minimized to facilitate rapid, high throughput screening, and typically are between 1 minute and 10 hours.

After incubation, the level of TLR signaling is detected using any suitable method. For cell-free binding type assays, a separation step is often used to separate bound from unbound components. The separation step may be accomplished in a variety of ways. For example, separation can be accomplished in solution, or, conveniently, at least one of the components is immobilized on a solid substrate, from which the unbound components may be readily separated. The solid substrate can be made using any of a wide variety of materials and in any of a wide variety of shapes, e.g., microtiter plate, microbead, dipstick, resin particle, etc. The substrate preferably is chosen to maximize signal-to-noise ratios, primarily to minimize background binding, as well as for ease of separation and cost.

Separation may be effected, for example, by removing a bead or dipstick from a reservoir, emptying or diluting a reservoir such as a microtiter plate well, rinsing a bead, particle, chromatographic column or filter with a wash solution or solvent. The separation step can include multiple rinses or washes. For example, when the solid substrate is a microtiter plate, the wells may be washed several times with a washing solution, which typically includes those components of the incubation mixture that do not participate in specific bindings such as salts, buffer, detergent, non-specific protein, etc. Where the solid substrate is a magnetic bead, the beads may be washed one or more times with a washing solution and isolated using a magnet.

Detection may be effected using any suitable method for cell-based assays such as measurement of an induced polypeptide within, on the surface of, or secreted by the cell. Examples of detection methods useful in cell-based assays include fluorescence-activated cell sorting (FACS) analysis, bioluminescence, fluorescence, enzyme-linked immunosorbent assay (ELISA), reverse transcriptase-polymerase chain reaction (RT-PCR), and the like. Alternatively, detection may be effected using any suitable method for cell-free assays. Examples of detection methods useful in cell-free assays include surface plasmon resonance, bioluminescence, fluorescence, ELISA, RT-PCR, and the like.

For use in therapy, different doses may be necessary for treatment of a subject, depending on activity of the compound, manner of administration, purpose of the immunization (i.e., prophylactic or therapeutic), nature and severity of the disorder, age and body weight of the subject. The administration of a given dose can be carried out both by single administration in the form of an individual dose unit or else several smaller dose units. Multiple administration of doses at specific intervals of weeks or months apart is usual for boosting antigen-specific immune responses.

Combined with the teachings provided herein, by choosing among the various active compounds and weighing factors such as potency, relative bioavailability, patient body weight, severity of adverse side-effects and preferred mode of administration, an effective prophylactic or therapeutic treatment regimen can be planned which does not cause substantial toxicity and yet is entirely effective to treat the particular subject. The effective amount for any particular application can vary depending on such factors as the disease or condition being treated, the particular therapeutic agent being administered (e.g., in the case of an immunostimulatory nucleic acid, the type of nucleic acid, i.e., a CpG nucleic acid, the number of unmethylated CpG motifs or their location in the nucleic acid, the degree of modification of the backbone to the oligonucleotide, etc.), the size of the subject, or the severity of the disease or condition. One of ordinary skill in the art can empirically determine the effective amount of a particular nucleic acid and/or other therapeutic agent without necessitating undue experimentation.

Subject doses of the compounds described herein typically range from about 0.1 μg to 10,000 mg, more typically from about 1 μg/day to 8000 mg, and most typically from about 10 μg to 100 μg. Stated in terms of subject body weight, typical dosages range from about 0.1 μg to 20 mg/kg/day, more typically from about 1 to 10 mg/kg/day, and most typically from about 1 to 5 mg/kg/day.

The pharmaceutical compositions containing nucleic acids and/or other compounds can be administered by any suitable route for administering medications. A variety of administration routes are available. The particular mode selected will depend, of course, upon the particular agent or agents selected, the particular condition being treated, and the dosage required for therapeutic efficacy. The methods of this invention, generally speaking, may be practiced using any mode of administration that is medically acceptable, meaning any mode that produces effective levels of an immune response without causing clinically unacceptable adverse effects. Preferred modes of administration are discussed herein. For use in therapy, an effective amount of the nucleic acid and/or other therapeutic agent can be administered to a subject by any mode that delivers the agent to the desired surface, e.g., mucosal, systemic.

Administering the pharmaceutical composition of the present invention may be accomplished by any means known to the skilled artisan. Routes of administration include but are not limited to oral, parenteral, intravenous, intramuscular, intranasal, sublingual, intratracheal, inhalation, subcutaneous, ocular, vaginal, and rectal. For the treatment or prevention of asthma or allergy, such compounds are preferably inhaled, ingested or administered by systemic routes. Systemic routes include oral and parenteral. Inhaled medications are preferred in some embodiments because of the direct delivery to the lung, the site of inflammation, primarily in asthmatic patients. Several types of devices are regularly used for administration by inhalation. These types of devices include metered dose inhalers (MDI), breath-actuated MDI, dry powder inhaler (DPI), spacer/holding chambers in combination with MDI, and nebulizers.

The therapeutic agents of the invention may be delivered to a particular tissue, cell type, or to the immune system, or both, with the aid of a vector. In its broadest sense, a “vector” is any vehicle capable of facilitating the transfer of the compositions to the target cells. The vector generally transports the immunostimulatory nucleic acid, antibody, antigen, and/or disorder-specific medicament to the target cells with reduced degradation relative to the extent of degradation that would result in the absence of the vector.

In general, the vectors useful in the invention are divided into two classes: biological vectors and chemical/physical vectors. Biological vectors and chemical/physical vectors are useful in the delivery and/or uptake of therapeutic agents of the invention.

Most biological vectors are used for delivery of nucleic acids and this would be most appropriate in the delivery of therapeutic agents that are or that include immunostimulatory nucleic acids.

In addition to the biological vectors discussed herein, chemical/physical vectors may be used to deliver therapeutic agents including immunostimulatory nucleic acids, antibodies, antigens, and disorder-specific medicaments. As used herein, a “chemical/physical vector” refers to a natural or synthetic molecule, other than those derived from bacteriological or viral sources, capable of delivering the nucleic acid and/or other medicament.

A preferred chemical/physical vector of the invention is a colloidal dispersion system. Colloidal dispersion systems include lipid-based systems including oil-in-water emulsions, micelles, mixed micelles, and liposomes. A preferred colloidal system of the invention is a liposome. Liposomes are artificial membrane vessels which are useful as a delivery vector in vivo or in vitro. It has been shown that large unilamellar vesicles (LUVs), which range in size from 0.2-4.0 μm can encapsulate large macromolecules. RNA, DNA and intact virions can be encapsulated within the aqueous interior and be delivered to cells in a biologically active form. Fraley et al. (1981) Trends Biochem Sci 6:77.

Liposomes may be targeted to a particular tissue by coupling the liposome to a specific ligand such as a monoclonal antibody, sugar, glycolipid, or protein. Ligands which may be useful for targeting a liposome to an immune cell include, but are not limited to: intact or fragments of molecules which interact with immune cell specific receptors and molecules, such as antibodies, which interact with the cell surface markers of immune cells. Such ligands may easily be identified by binding assays well known to those of skill in the art. In still other embodiments, the liposome may be targeted to the cancer by coupling it to a one of the immunotherapeutic antibodies discussed earlier. Additionally, the vector may be coupled to a nuclear targeting peptide, which will direct the vector to the nucleus of the host cell.

Lipid formulations for transfection are commercially available from QIAGEN, for example, as EFFECTENE™ (a non-liposomal lipid with a special DNA condensing enhancer) and SUPERFECT™ (a novel acting dendrimeric technology).

Liposomes are commercially available from Gibco BRL, for example, as LIPOFECTIN™ and LIPOFECTACE™, which are formed of cationic lipids such as N-[1-(2,3dioleyloxy)-propyl]-N,N,N-trimethylammonium chloride (DOTMA) and dimethyl dioctadecylammonium bromide (DDAB). Methods for making liposomes are well known in the art and have been described in many publications. Liposomes also have been reviewed by Gregoriadis G (1985) Trends Biotechnol 3:235-241.

Certain cationic lipids, including in particular N-[1-(2,3dioleoyloxy)-propyl]-N,N,N-trimethylammonium methyl-sulfate (DOTAP), appear to be especially advantageous when combined with the modified oligoribonucleotide analogs of the invention.

In one embodiment, the vehicle is a biocompatible microparticle or implant that is suitable for implantation or administration to the mammalian recipient. Exemplary bioerodible implants that are useful in accordance with this method are described in PCT International application no. PCT/US/03307 (Publication No. WO95/24929, entitled “Polymeric Gene Delivery System”. PCT/US/0307 describes a biocompatible, preferably biodegradable polymeric matrix for containing an exogenous gene under the control of an appropriate promoter. The polymeric matrix can be used to achieve sustained release of the therapeutic agent in the subject.

The polymeric matrix preferably is in the form of a microparticle such as a microsphere (wherein the nucleic acid and/or the other therapeutic agent is dispersed throughout a solid polymeric matrix) or a microcapsule (wherein the nucleic acid and/or the other therapeutic agent is stored in the core of a polymeric shell). Other forms of the polymeric matrix for containing the therapeutic agent include films, coatings, gels, implants, and stents. The size and composition of the polymeric matrix device is selected to result in favorable release kinetics in the tissue into which the matrix is introduced. The size of the polymeric matrix further is selected according to the method of delivery which is to be used, typically injection into a tissue or administration of a suspension by aerosol into the nasal and/or pulmonary areas. Preferably when an aerosol route is used the polymeric matrix and the nucleic acid and/or the other therapeutic agent are encompassed in a surfactant vehicle. The polymeric matrix composition can be selected to have both favorable degradation rates and also to be formed of a material which is bioadhesive, to further increase the effectiveness of transfer when the matrix is administered to a nasal and/or pulmonary surface that has sustained an injury. The matrix composition also can be selected not to degrade, but rather, to release by diffusion over an extended period of time. In some preferred embodiments, the nucleic acid are administered to the subject via an implant while the other therapeutic agent is administered acutely. Biocompatible microspheres that are suitable for delivery, such as oral or mucosal delivery, are disclosed in Chickering et al. (1996) Biotech Bioeng 52:96-101 and Mathiowitz E et al. (1997) Nature 386:410-414 and PCT Pat. Application WO97/03702.

Both non-biodegradable and biodegradable polymeric matrices can be used to deliver the nucleic acid and/or the other therapeutic agent to the subject. Biodegradable matrices are preferred. Such polymers may be natural or synthetic polymers. The polymer is selected based on the period of time over which release is desired, generally in the order of a few hours to a year or longer. Typically, release over a period ranging from between a few hours and three to twelve months is most desirable, particularly for the nucleic acid agents. The polymer optionally is in the form of a hydrogel that can absorb up to about 90% of its weight in water and further, optionally is cross-linked with multi-valent ions or other polymers.

Bioadhesive polymers of particular interest include bioerodible hydrogels described by H. S. Sawhney, C. P. Pathak and J. A. Hubell in Macromolecules, (1993) 26:581-587, the teachings of which are incorporated herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

If the therapeutic agent is a nucleic acid, the use of compaction agents may also be desirable. Compaction agents also can be used alone, or in combination with, a biological or chemical/physical vector. A “compaction agent”, as used herein, refers to an agent, such as a histone, that neutralizes the negative charges on the nucleic acid and thereby permits compaction of the nucleic acid into a fine granule. Compaction of the nucleic acid facilitates the uptake of the nucleic acid by the target cell. The compaction agents can be used alone, i.e., to deliver a nucleic acid in a form that is more efficiently taken up by the cell or, more preferably, in combination with one or more of the above-described vectors.

Other exemplary compositions that can be used to facilitate uptake of a nucleic acid include calcium phosphate and other chemical mediators of intracellular transport, microinjection compositions, electroporation and homologous recombination compositions (e.g., for integrating a nucleic acid into a preselected location within the target cell chromosome).

The compounds may be administered alone (e.g., in saline or buffer) or using any delivery vehicle known in the art. For instance the following delivery vehicles have been described: cochleates (Gould-Fogerite et al., 1994, 1996); Emulsomes (Vancott et al., 1998, Lowell et al., 1997); ISCOMs (Mowat et al., 1993, Carlsson et al., 1991, Hu et., 1998, Morein et al., 1999); liposomes (Childers et al., 1999, Michalek et al., 1989, 1992, de Haan 1995a, 1995b); live bacterial vectors (e.g., Salmonella, Escherichia coli, Bacillus Calmette-Guérin, Shigella, Lactobacillus) (Hone et al., 1996, Pouwels et al., 1998, Chatfield et al., 1993, Stover et al., 1991, Nugent et al., 1998); live viral vectors (e.g., Vaccinia, adenovirus, Herpes Simplex) (Gallichan et al., 1993, 1995, Moss et al., 1996, Nugent et al., 1998, Flexner et al., 1988, Morrow et al., 1999); microspheres (Gupta et al., 1998, Jones et al., 1996, Maloy et al., 1994, Moore et al., 1995, O'Hagan et al., 1994, Eldridge et al., 1989); nucleic acid vaccines (Fynan et al., 1993, Kuklin et al., 1997, Sasaki et al., 1998, Okada et al., 1997, Ishii et al., 1997); polymers (e.g. carboxymethylcellulose, chitosan) (Hamajima et al., 1998, Jabbal-Gill et al., 1998); polymer rings (Wyatt et al., 1998); proteosomes (Vancott et al., 1998, Lowell et al., 1988, 1996, 1997); sodium fluoride (Hashi et al., 1998); transgenic plants (Tacket et al., 1998, Mason et al., 1998, Haq et al., 1995); virosomes (Gluck et al., 1992, Mengiardi et al., 1995, Cryz et al., 1998); and, virus-like particles (Jiang et al., 1999, Leibl et al., 1998).

The formulations of the invention are administered in pharmaceutically acceptable solutions, which may routinely contain pharmaceutically acceptable concentrations of salt, buffering agents, preservatives, compatible carriers, adjuvants, and optionally other therapeutic ingredients.

The term pharmaceutically-acceptable carrier means one or more compatible solid or liquid filler, diluents or encapsulating substances which are suitable for administration to a human or other vertebrate animal. The term carrier denotes an organic or inorganic ingredient, natural or synthetic, with which the active ingredient is combined to facilitate the application. The components of the pharmaceutical compositions also are capable of being commingled with the compounds of the present invention, and with each other, in a manner such that there is no interaction which would substantially impair the desired pharmaceutical efficiency.

For oral administration, the compounds (i.e., nucleic acids, antigens, antibodies, and other therapeutic agents) can be formulated readily by combining the active compound(s) with pharmaceutically acceptable carriers well known in the art. Such carriers enable the compounds of the invention to be formulated as tablets, pills, dragees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers for neutralizing internal acid conditions or may be administered without any carriers.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds for use according to the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active compounds may be in powder form for constitution with a suitable vehicle, e.g., sterile pyrogen-free water, before use.

The compounds may also be formulated in rectal or vaginal compositions such as suppositories or retention enemas, e.g., containing conventional suppository bases such as cocoa butter or other glycerides.

In addition to the formulations described previously, the compounds may also be formulated as a depot preparation. Such long-acting formulations may be formulated with suitable polymeric or hydrophobic materials (for example as an emulsion in an acceptable oil) or ion exchange resins, or as sparingly soluble derivatives, for example, as a sparingly soluble salt.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer R (1990) Science 249:1527-1533, which is incorporated herein by reference.

The nucleic acids and optionally other therapeutics and/or antigens may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

The compositions may conveniently be presented in unit dosage form and may be prepared by any of the methods well known in the art of pharmacy. All methods include the step of bringing the compounds into association with a carrier which constitutes one or more accessory ingredients. In general, the compositions are prepared by uniformly and intimately bringing the compounds into association with a liquid carrier, a finely divided solid carrier, or both, and then, if necessary, shaping the product. Liquid dose units are vials or ampoules. Solid dose units are tablets, capsules and suppositories.

Other delivery systems can include time-release, delayed release or sustained release delivery systems. Such systems can avoid repeated administrations of the compounds, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono-, di-, and tri-glycerides; hydrogel release systems; silastic systems; peptide-based systems; wax coatings; compressed tablets using conventional binders and excipients; partially fused implants; and the like. Specific examples include, but are not limited to: (a) erosional systems in which an agent of the invention is contained in a form within a matrix such as those described in U.S. Pat. Nos. 4,452,775, 4,675,189, and 5,736,152, and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation.

The present invention is further illustrated by the following Examples, which in no way should be construed as further limiting.

### EXAMPLES

### Example 1

### Influence of Sulfur and Triethylene Glycol Modifications on Cytokine Production by Human Peripheral Blood Mononuclear Cells

Human peripheral blood mononuclear cells (PBMC) were isolated from three donors and incubated for 24 hours in the presence of various test or control oligonucleotides or control conditions, in either the presence or absence of DOTAP (20 μg/ml). Oligonucleotides were added at different concentrations ranging from 0.001-4 μM. Culture supernatants were then collected and then analyzed by separate enzyme-linked immunosorbent assays (ELISAs) specific for human interferon alpha (IFN-α) and tumor necrosis factor alpha (TNF-α).

Control oligonucleotides and conditions included R-1012, rU*rU*rU*rU*rU*rU*rU*rU, where * represents phosphorothioate linkage and rU represents uridine; R-1075 (SEQ ID NO:206, fully phosphorothioate backbone), R-1362, rU*rU*rG*rU*rU*rG*rU*rU*rG*rU*rU*rG*rU*rU*rG*rU*rU*rG*rU*rU (SEQ ID NO:331), where * again represents phosphorothioate linkage, rU again represents uridine, and rG represents guanosine; CpG oligodeoxynucleotide 2395, T*C*G*T*C*G*T*T*T*T*C*G*G*C*G*C*G*C*G*C*C*G (SEQ ID NO:330), lipopolysaccharide (LPS), DOTAP, and no additive (w/o).

Test oligonucleotides included the following modifications of R-1012: R-1907, rU*rU*rU*rU*rU*rU*rU*rU-teg, where teg represents triethylene glycol; R-1908, rU*rU*rU*rU*rU*rU*rU*5SrU-teg, where 5S represents a linkage according to Formula II wherein X is O, X¹ is SH, X² is O, and X³ is S; R-1909, rU*rU*rU*rU*rU*5SrU*rU*5SrU-teg; R-1910, rU*rU*rU*5SrU*rU*5SrU*rU*5SrU-teg; and R-1911, rU*5SrU*rU*5SrU*rU*5SrU*rU*5SrU-teg.

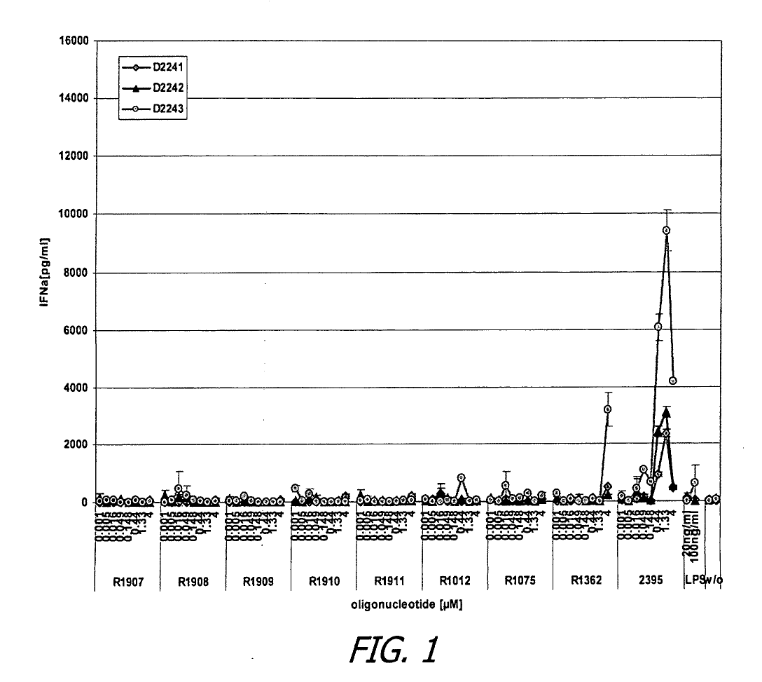

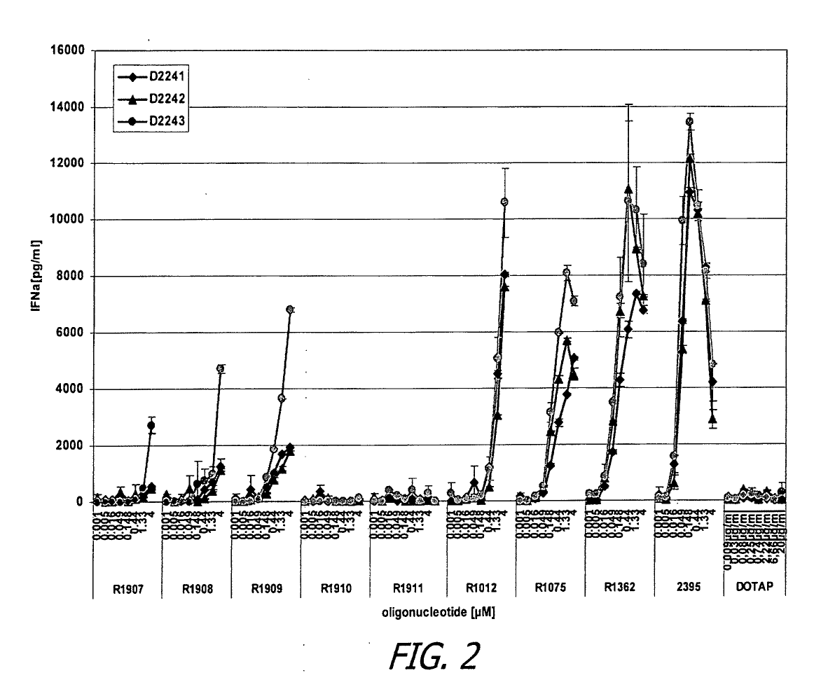

The results for IFN-α are depicted in FIG. 1 and FIG. 2. In the presence of DOTAP, R-1907 (3′ teg modification on R-1012) induced reduced IFN-α production compared to unmodified R-1012. Addition of sulfur modifications, in R-1908 and R-1909, resulted in an increase in IFN-α induction compared to R-1907. In this experiment R-1910 and R-1911 did not induce significant amounts of IFN-α.

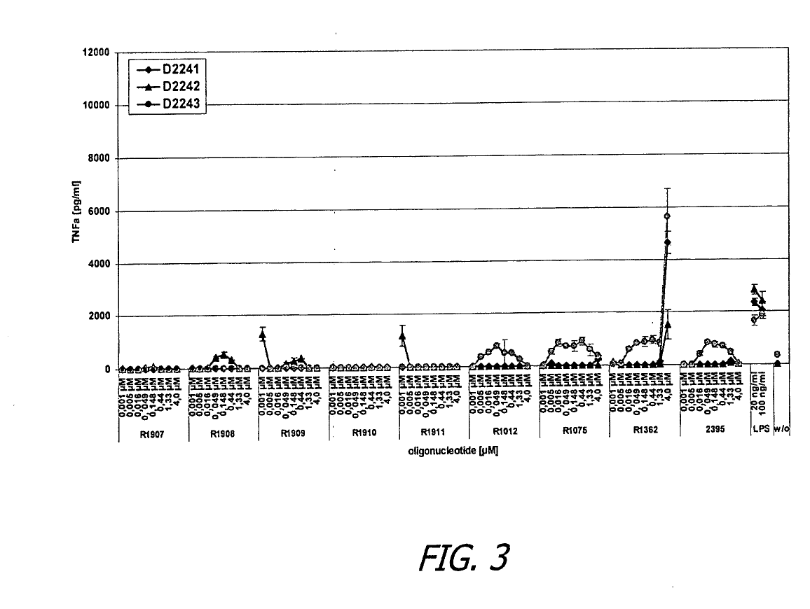

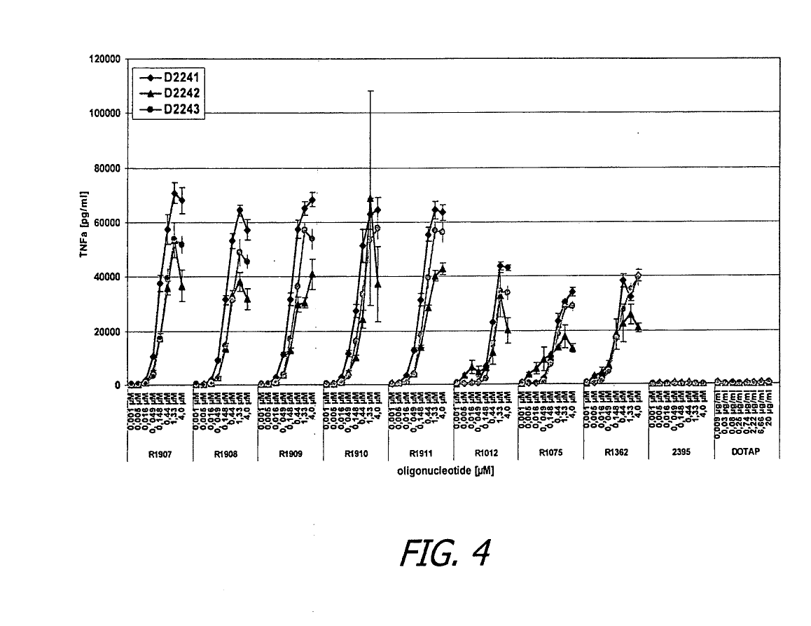

The results for TNF-α are depicted in FIG. 3 and FIG. 4. In the presence of DOTAP, R-1907 (3′ teg modification on R-1012) enhanced TNF-α production compared to unmodified R-1012. R-1907, R-1908, R-1909, R-1910, and R-1911 induced significantly higher amounts of TNF-a compared to unmodified R-1012.

### Example 2

### Triethylene Glycol Modification Stabilizes ORN Against Nuclease Degradation

Oligoribonucleotides R-1075 (SEQ ID NO:206), without triethylene glycol (teg) modification, and R-1907, with teg modification, were analysed using ion-pair reverse phase high pressure liquid chromatography (IP-RP-HPLC) following incubation in water or human serum for 1 to 60 minutes. While both of these oligoribonucleotides have fully phosphorothioate backbones, R-1075 is more than twice as long (18 nucleotides) as R-1907 (8 nucleotides). R-1075 incubated in water for 1 minute produced a single, sharp peak upon IP-RP-HPLC. In contrast, this peak essentially completely disappeared following incubation of R-1075 in human serum for just 1 minute. R-1907 also produced a single, sharp peak following incubation in water for 1 minute. In contrast to R-1075, however, this single, sharp peak for R-1907 persisted essentially unchanged following incubation in human serum for 1 minute. In fact, the peak height for R-1907 decreased by only about 50 percent following incubation in human serum for 60 minutes. These results indicated that triethylene glycol modification stabilized R-1907 against degradation by nucleases normally present in human serum.

### Example 3

### Preparation of 5′ Thiouridine-Containing Oligonucleotides

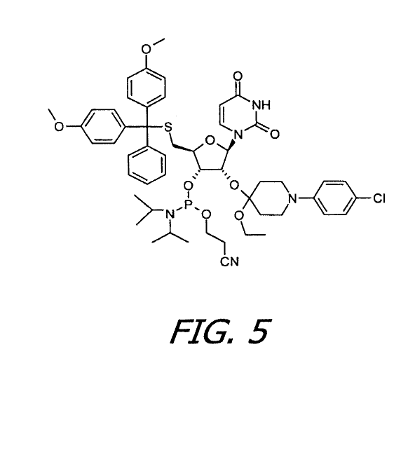

As described in Example 1 above, oligonucleotides R-1908, R-1909, R-1910, and R-1911 each contain at least one 5′ thiouridine residue according to Formula II wherein X is O, X¹ is SH, X² is O, and X³ is S. These oligonucleotides were prepared using standard chemistries to incorporate monomers of 5′-DMT-2′-O-Cpep-5′-thio-uridine-3′-phosphoramidite (FIG. 5), wherein Cpep is 1-(4-chlorophenyl)-4-ethoxypiperidin-4-yl and DMT is dimethoxytrityl.

### Equivalents

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages of the invention are not necessarily encompassed by each embodiment of the invention.

All references, patents and patent publications that are recited in this application are incorporated in their entirety herein by reference.

We claim:

1. An immunostimulatory composition comprising a polymer 4 to 100 units long, wherein each unit comprises a nucleoside or a nucleoside analog, wherein each pair of adjacent units is linked by a covalent linkage, and wherein the composition comprises
(a) an immunostimulatory RNA motif 4 to 8 nucleotides long, and
(b) at least one modified phosphate linkage selected from the group consisting of: 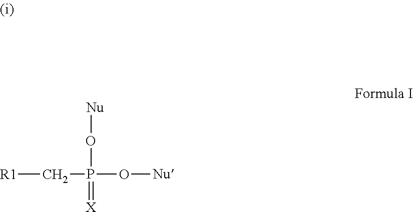
wherein
R1 is hydrogen (H), COOR, OH, C1-C18 alkyl, C₆H₅, or (CH₂)ₘ—NH—R2, wherein R is H or methyl, butyl, methoxyethyl, pivaloyl oxymethyl, pivaloyl oxybenzyl, or S-pivaloyl thioethyl; R2 is H, C1-C18 alkyl, or C2-C18 acyl; and m is 1 to 17;
X is oxygen (O) or sulfur (S); and
each of Nu and Nu′ independently is a nucleoside or nucleoside analog; with the proviso that if R1 is H, then X is S; 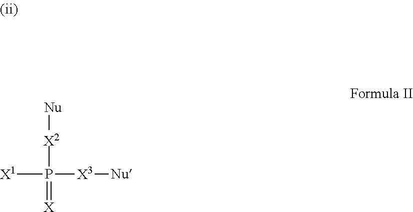
wherein
X is O or S;
X¹ is OH, SH, BH₃, OR3, or NHR3, wherein R3 is C1-C18 alkyl;
each of X² and X³ independently is O, S, CH₂, or CF₂; and
each of Nu and Nu′ independently is a nucleoside or nucleoside analog; with the proviso that
(a) at least one of X, X², and X³ is not O or X¹ is not OH,
(b) if X¹ is SH, then at least one of X, X², and X³ is not O,
(c) if X and X² are O and if X¹ is OH, then X³ is not S and Nu is 3′Nu and Nu′ is 5′Nu′, and
(d) if X¹ is BH₃, then at least one of X, X², or X³ is S; and
(iii) any combination of (i) and (ii).

2. The composition of claim 1, wherein the immunostimulatory RNA motif has a 15 base sequence selected from
(i) 5′-C/U-U-G/U-U-3′,
(ii) 5′-R-U-R-G-Y-3′,
(iii) 5′-G-U-U-G-B-3′,
(iv) 5′-G-U-G-U-G/U-3′, and
(v) 5′-G/C-U-A/C-G-G-C-A-C-3′,
wherein-C/U- is cytosine (C) or uracil (U), G/U- is guanine (G) or U, R is purine; Y is pyrimidine, B is U, G, or C, G/C is G or C, and A/C is adenine (A) or C.

3. The composition of claim 1, wherein the immunostimulatory RNA motif is 5′-C/U-U-G/U-U-3′.

4. The composition of claim 1, wherein the immunostimulatory RNA motif is 5’-R-U-R-G-Y-3′.

5. The composition of claim 1, wherein the immunostimulatory RNA motif is 5′-G-U-U-G-B-3′.

6. The composition of claim 1, wherein the immunostimulatory RNA motif is 5′-G-U-G-U-G/U-3′.

7. The composition of claim 1, wherein the immunostimulatory RNA motif is 5 5′-G/C-U-A/C-G-G-C-A-C-3′.

8-33. (canceled)

34. An immunostimulatory composition comprising a polymer 4 to 100 units long, wherein each unit comprises a nucleoside or a nucleoside analog, wherein each pair of adjacent units is linked by a covalent linkage, and wherein the composition comprises
(a) an immunostimulatory RNA motif 4 to 8 nucleotides long, and
(b) at least one nucleotide analog provided as Formula IIIA or Formula IIIB 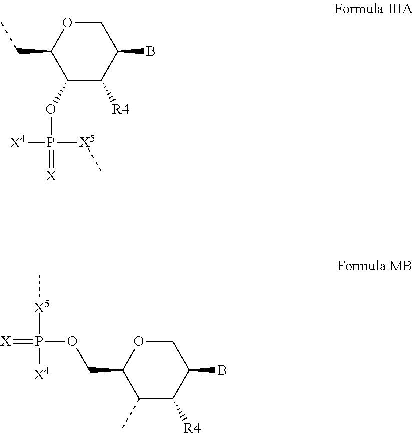
wherein
R4 is H or OR, wherein R is H or C1-C18 acyl;
B is a nucleobase, a modified nucleobase, or H;
each of X and X⁵ independently is O or S; and
X⁴ is OH, SH, methyl, or NHR5, wherein R5 is C1-C18 alkyl; and
each dashed line independently represents an optional bond to an adjacent unit, hydrogen, or an organic radical;
with the proviso that at least one of X and X⁵ is not O or X⁴ is not OH.

35-57. (canceled)

58. An immunostimulatory composition comprising the immunostimulatory composition of claim 1, further comprising at least one nucleotide analog provided as Formula IIIA or Formula IIIB 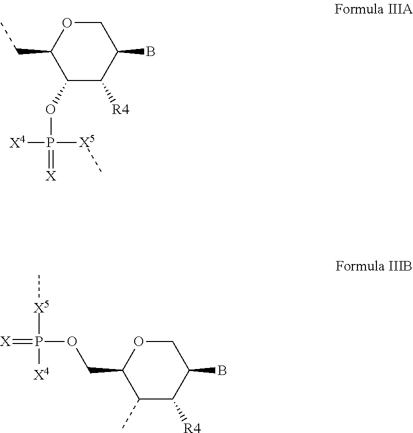
wherein
R4 is H or OR, wherein R is H or C1-C18 acyl;
B is a nucleobase, a modified nucleobase, or H;
each of X and X⁵ independently is O or S; and
X⁴ is OH, SH, methyl, or NHR5, wherein R5 is C1-C18 alkyl; and
each dashed line independently represents an optional bond to an adjacent unit, hydrogen, or an organic radical;
with the proviso that at least one of X and X⁵ is not O or X⁴ is not OH.

59-87. (canceled)

88. A pharmaceutical composition of claim 1, in association with a delivery vehicle chosen from a cationic lipid, a liposome, a cochleate, a virosome, an immune-stimulating complex (ISCOM), a microparticle, a microsphere, a nanosphere, a unilamellar vesicle (LUV), a multilarnellar vesicle, an oil-in-water emulsion, a water-in oil emulsion, an emulsome, and a polycationic peptide, and, optionally, a pharmaceutically acceptable carrier.

89. (canceled)

90. A method of activating an immune cell, the method comprising contacting an 20 immune cell with an effective amount of the composition of claim 1.

91. A method of vaccinating a subject, the method comprising administering to the subject an antigen and a composition of claim 1.

92-98. (canceled)

99. A method of treating a subject having an infection, the method comprising administering to the subject an effective amount of the composition of claim 1.

100. (canceled)

101. A method of treating a subject having a cancer, the method comprising administering to the subject an effective amount of the composition of claim 1.

102. (canceled)

103. A method of treating a subject having an allergic condition, the method comprising administering to the subject an effective amount of the composition of claim 1.

104. (canceled)

105. A method of treating a subject having asthma, the method comprising administering to the subject an effective amount of the composition of claim 1.

106. (canceled)

107. A method for treating a subject having airway remodeling, the method comprising administering to the subject an effective amount of an immunostimulatory composition of claim 1.

108. A method for increasing antibody-dependent cellular cytotoxicity (ADCC), the method comprising administering to a subject in need of increased ADCC an effective amount of an immunostimulatory composition of claim 1, to increase ADCC.

109-110. (canceled)

111. A method for enhancing epitope spreading, the method comprising contacting a cell of the immune system with an antigen and subsequently contacting the cell with at least two doses of an immunostimulatory composition of claim 1.

112. (canceled)

113. A method for enhancing epitope spreading in a subject, the method comprising administering to the subject a vaccine comprising an antigen and an adjuvant and subsequently administering to the subject at least two doses of an isolated immunostimulatory composition of claim 1, in an effective amount to induce multiple epitope-specific immune responses.

114. (canceled)

115. A method for enhancing epitope spreading in a subject, the method comprising applying a therapeutic protocol which results in immune system antigen exposure in the subject and subsequently administering at least two doses of an isolated immunostimulatory composition of claim 1, in an effective amount to induce multiple epitope-specific immune responses.

116. (canceled)


SEQUENCE LISTING (331 sequences)

SEQ ID NO: 1 - RNA, 30 nt, Artificial sequence
  Synthetic oligonucleotide
uuugugugucucucuuguuuuugugugucu

SEQ ID NO: 2 - RNA, 30 nt, Artificial sequence
  Synthetic oligonucleotide
uuuccaaacaagucucuucucuuguuuggu

SEQ ID NO: 3 - RNA, 30 nt, Artificial sequence
  Synthetic oligonucleotide
uuuaucuauccuuagccaacuuugucuggu

SEQ ID NO: 4 - RNA, 30 nt, Artificial sequence
  Synthetic oligonucleotide
uuuaucuauccauagccaacuuuuucuggu

SEQ ID NO: 5 - RNA, 30 nt, Artificial sequence
  Synthetic oligonucleotide
uuuaacuauccuuagccaacuuugucuggu

SEQ ID NO: 6 - RNA, 30 nt, Artificial sequence
  Synthetic oligonucleotide
uugucauauaauugguuuuuuugucuucgu

SEQ ID NO: 7 - RNA, 30 nt, Artificial sequence
  Synthetic oligonucleotide
uuguauucauuuuaaacuccugcuuuugcu

SEQ ID NO: 8 - RNA, 30 nt, Artificial sequence
  Synthetic oligonucleotide
uuguauucauuuuaaaccccugcuuuugcu

SEQ ID NO: 9 - RNA, 30 nt, Artificial sequence
  Synthetic oligonucleotide
uuguauuaggaaugguuuuuuugucuucgu

SEQ ID NO: 10 - RNA, 30 nt, Artificial sequence
  Synthetic oligonucleotide
uuggauucauuuuaaucuccugcuuuugcu

SEQ ID NO: 11 - RNA, 30 nt, Artificial sequence
  Synthetic oligonucleotide
uugaucuauccuuacccaacuuuguuuggu

SEQ ID NO: 12 - RNA, 30 nt, Artificial sequence
  Synthetic oligonucleotide
uugaacuauccuuacccaacuuuguuuggu

SEQ ID NO: 13 - RNA, 30 nt, Artificial sequence
  Synthetic oligonucleotide
uucccagacaaguuucuucucuuguuuggu

SEQ ID NO: 14 - RNA, 30 nt, Artificial sequence
  Synthetic oligonucleotide
uucccaagcaagucucuucucuuguuuggu

SEQ ID NO: 15 - RNA, 30 nt, Artificial sequence
  Synthetic oligonucleotide
uuccauuuuggaucaguaccugcuuuugcu

SEQ ID NO: 16 - RNA, 30 nt, Artificial sequence
  Synthetic oligonucleotide
uuccauuuuggaucaguaccugcuuucgcu

SEQ ID NO: 17 - RNA, 30 nt, Artificial sequence
  Synthetic oligonucleotide
uuccauuuugaaucaguaccugcuuucgcu

SEQ ID NO: 18 - RNA, 30 nt, Artificial sequence; identical to SEQ ID NO: 17
  Synthetic oligonucleotide

SEQ ID NO: 19 - RNA, 30 nt, Artificial sequence
  Synthetic oligonucleotide
uuccauuucggaucaguaccugcuuuugcu

SEQ ID NO: 20 - RNA, 30 nt, Artificial sequence
  Synthetic oligonucleotide
uuccauuucgaaucaguaccugcuuucgcu

SEQ ID NO: 21 - RNA, 30 nt, Artificial sequence
  Synthetic oligonucleotide
uuccauucugaaucaguaccugcuuuugcu

SEQ ID NO: 22 - RNA, 30 nt, Artificial sequence
  Synthetic oligonucleotide
uuauggcaaaucaaacguaucgcuucugcu

SEQ ID NO: 23 - RNA, 30 nt, Artificial sequence
  Synthetic oligonucleotide
uuauggcaaaucaaacgcaccgcuucugcu

SEQ ID NO: 24 - RNA, 30 nt, Artificial sequence
  Synthetic oligonucleotide
uuaucguaccuuacagauucucuguuuggu

SEQ ID NO: 25 - RNA, 30 nt, Artificial sequence
  Synthetic oligonucleotide
uuaucguaccucacagauucucuguuuggu

SEQ ID NO: 26 - RNA, 30 nt, Artificial sequence
  Synthetic oligonucleotide
uuaucguaacuuacggauucucuguuuggu

SEQ ID NO: 27 - RNA, 30 nt, Artificial sequence
  Synthetic oligonucleotide
uuaucguaacucacggauucucuguuuggu

SEQ ID NO: 28 - RNA, 30 nt, Artificial sequence
  Synthetic oligonucleotide
uuaucguaacucaccgauucucuguuuggu

SEQ ID NO: 29 - RNA, 30 nt, Artificial sequence
  Synthetic oligonucleotide
uuauauucaucuuaaagcuccgcuucugcu

SEQ ID NO: 30 - RNA, 30 nt, Artificial sequence
  Synthetic oligonucleotide
uuaccaagcaaguuucuucucuuguuuggu

SEQ ID NO: 31 - RNA, 30 nt, Artificial sequence
  Synthetic oligonucleotide
uguuuuuucuuugaucugguuguuaagcgu

SEQ ID NO: 32 - RNA, 30 nt, Artificial sequence
  Synthetic oligonucleotide
ugugucuucuuugaucugguuguuaagcgu

SEQ ID NO: 33 - RNA, 30 nt, Artificial sequence
  Synthetic oligonucleotide
uguaacauaacucaucaucuuuuaugauac

SEQ ID NO: 34 - RNA, 30 nt, Artificial sequence
  Synthetic oligonucleotide
ugguuguuuuuauuuuccccugcuuuugcu

SEQ ID NO: 35 - RNA, 30 nt, Artificial sequence
  Synthetic oligonucleotide
ugguuguauuuauuuuccccugcuuuugcu

SEQ ID NO: 36 - RNA, 30 nt, Artificial sequence
  Synthetic oligonucleotide
ugguugguuuuauuuuccccugcuuuugcu

SEQ ID NO: 37 - RNA, 30 nt, Artificial sequence
  Synthetic oligonucleotide
ugguugcuuuuauuuuccccugcuuuugcu

SEQ ID NO: 38 - RNA, 30 nt, Artificial sequence
  Synthetic oligonucleotide
ugguugauuuuauuuuccccugcuuuugcu

SEQ ID NO: 39 - RNA, 30 nt, Artificial sequence
  Synthetic oligonucleotide
ugguugauuugauuucccccugcuuuugcu

SEQ ID NO: 40 - RNA, 30 nt, Artificial sequence
  Synthetic oligonucleotide
ugguugauuuaauuuuccccugcuuuugcu

SEQ ID NO: 41 - RNA, 30 nt, Artificial sequence
  Synthetic oligonucleotide
ugcuucuucuuugguuuuguuguuaagcgu

SEQ ID NO: 42 - RNA, 30 nt, Artificial sequence
  Synthetic oligonucleotide
ugcaaguuuguuguacgcauuuuuucccgu

SEQ ID NO: 43 - RNA, 30 nt, Artificial sequence
  Synthetic oligonucleotide
ugcaaguuuguaguacgcauuuuuucgcgu

SEQ ID NO: 44 - RNA, 30 nt, Artificial sequence; identical to SEQ ID NO: 43
  Synthetic oligonucleotide

SEQ ID NO: 45 - RNA, 30 nt, Artificial sequence
  Synthetic oligonucleotide
ugauuuuuauaugguuuuuuuguuaagcgu

SEQ ID NO: 46 - RNA, 30 nt, Artificial sequence
  Synthetic oligonucleotide
ucuuccaaguaucaucaucuuuuuugauac

SEQ ID NO: 47 - RNA, 30 nt, Artificial sequence
  Synthetic oligonucleotide
uauccaucuugaaaauagccaaucuuagcu

SEQ ID NO: 48 - RNA, 30 nt, Artificial sequence
  Synthetic oligonucleotide
uauauucaucuuaaaggcuccgcuucugcu

SEQ ID NO: 49 - RNA, 30 nt, Artificial sequence
  Synthetic oligonucleotide
uauaccuauccuuacccagcuuuguuuggu

SEQ ID NO: 50 - RNA, 30 nt, Artificial sequence
  Synthetic oligonucleotide
uagaccgauccuuacccaacuuuguuuggu

SEQ ID NO: 51 - RNA, 30 nt, Artificial sequence
  Synthetic oligonucleotide
uagaacgauccuuacccagcuuugucuggu

SEQ ID NO: 52 - RNA, 30 nt, Artificial sequence
  Synthetic oligonucleotide
uaauuguaauaaugguuuuuuugucuucgu

SEQ ID NO: 53 - RNA, 30 nt, Artificial sequence
  Synthetic oligonucleotide
uaauuguaagaaugguuuuuuugucuucgu

SEQ ID NO: 54 - RNA, 30 nt, Artificial sequence
  Synthetic oligonucleotide
uaauuauauuaaugguuuguuugucuucgu

SEQ ID NO: 55 - RNA, 30 nt, Artificial sequence
  Synthetic oligonucleotide
uaauguuaucaaugguuuauuugucuucgu

SEQ ID NO: 56 - RNA, 30 nt, Artificial sequence
  Synthetic oligonucleotide
uaaugguaauaaugguuuguuugucuucgu

SEQ ID NO: 57 - RNA, 30 nt, Artificial sequence
  Synthetic oligonucleotide
uaaugauaauaaugguuuguuugucuucgu

SEQ ID NO: 58 - RNA, 30 nt, Artificial sequence
  Synthetic oligonucleotide
uaagaaugcuauugguuuguuuuucuucgu

SEQ ID NO: 59 - RNA, 30 nt, Artificial sequence
  Synthetic oligonucleotide
uaacuuaauuuauacgcguuuuuuucgcgu

SEQ ID NO: 60 - RNA, 30 nt, Artificial sequence
  Synthetic oligonucleotide
uaaaaauucuucuuucuuuuuguguguccg

SEQ ID NO: 61 - RNA, 30 nt, Artificial sequence
  Synthetic oligonucleotide
uaaaaaaccuuuuuucuuuuuguguguccg

SEQ ID NO: 62 - RNA, 30 nt, Artificial sequence
  Synthetic oligonucleotide
guugcuuuuauuuuccccugcuuuugcuaa

SEQ ID NO: 63 - RNA, 30 nt, Artificial sequence
  Synthetic oligonucleotide
guggauauuagaaaaugcucugcuucugcu

SEQ ID NO: 64 - RNA, 30 nt, Artificial sequence
  Synthetic oligonucleotide
gguugcuuuuauuuuccccugcuuuugcua

SEQ ID NO: 65 - RNA, 30 nt, Artificial sequence
  Synthetic oligonucleotide
ggauucauuuugaacuccugcuuuugcuaa

SEQ ID NO: 66 - RNA, 30 nt, Artificial sequence
  Synthetic oligonucleotide
ggauacauaucucuuaaacucuugucuggu

SEQ ID NO: 67 - RNA, 30 nt, Artificial sequence
  Synthetic oligonucleotide
cuuuucuucucugguuuuguuguuaagcgu

SEQ ID NO: 68 - RNA, 30 nt, Artificial sequence
  Synthetic oligonucleotide
cugagcuuagucaaguuacuuuuuuuauac

SEQ ID NO: 69 - RNA, 30 nt, Artificial sequence
  Synthetic oligonucleotide
cugagcuuagucaaguuacuuuucuuauac

SEQ ID NO: 70 - RNA, 30 nt, Artificial sequence
  Synthetic oligonucleotide
cucaucuuucaauaucuaccugcuuuugcu

SEQ ID NO: 71 - RNA, 30 nt, Artificial sequence
  Synthetic oligonucleotide
cucaucuuucaauaucuaccugcuuucgcu

SEQ ID NO: 72 - RNA, 30 nt, Artificial sequence
  Synthetic oligonucleotide
cucaucuuucaacaucuaccugcuuuugcu

SEQ ID NO: 73 - RNA, 30 nt, Artificial sequence
  Synthetic oligonucleotide
cuaaaaauucuucuuucuuuuugugugccc

SEQ ID NO: 74 - RNA, 30 nt, Artificial sequence
  Synthetic oligonucleotide
cggugagugauuaucuacccugcuuuugcu

SEQ ID NO: 75 - RNA, 30 nt, Artificial sequence
  Synthetic oligonucleotide
cggugagagauuaucuacccugcuuuugcu

SEQ ID NO: 76 - RNA, 30 nt, Artificial sequence; identical to SEQ ID NO: 75
  Synthetic oligonucleotide

SEQ ID NO: 77 - RNA, 30 nt, Artificial sequence
  Synthetic oligonucleotide
cgcaaguuuguuguacgcauuuuuucgcgu

SEQ ID NO: 78 - RNA, 30 nt, Artificial sequence
  Synthetic oligonucleotide
ccgauaucccaucuucuuuuuccccuuggu

SEQ ID NO: 79 - RNA, 30 nt, Artificial sequence
  Synthetic oligonucleotide
ccauuaugucuuugucacccugcuuuugcu

SEQ ID NO: 80 - RNA, 30 nt, Artificial sequence
  Synthetic oligonucleotide
ccaauaucccaucuucauuuuccccuuggu

SEQ ID NO: 81 - RNA, 30 nt, Artificial sequence
  Synthetic oligonucleotide
ccaauaucccauauucauucuccccuuggu

SEQ ID NO: 82 - RNA, 30 nt, Artificial sequence
  Synthetic oligonucleotide
ccaacaucccaucuucuuuuuccccuuggu

SEQ ID NO: 83 - RNA, 30 nt, Artificial sequence
  Synthetic oligonucleotide
cauugagugauuaucuacccugcuuuugcu

SEQ ID NO: 84 - RNA, 30 nt, Artificial sequence
  Synthetic oligonucleotide
cauauugaauauaauugcgcugcuuucgcu

SEQ ID NO: 85 - RNA, 30 nt, Artificial sequence
  Synthetic oligonucleotide
cauauugaauauaauugaccugcuuucgcu

SEQ ID NO: 86 - RNA, 30 nt, Artificial sequence
  Synthetic oligonucleotide
cauauucaauauaauugaccugcuuuucgu

SEQ ID NO: 87 - RNA, 30 nt, Artificial sequence
  Synthetic oligonucleotide
cagugagugauuauuaacccugcuuuugcu

SEQ ID NO: 88 - RNA, 30 nt, Artificial sequence
  Synthetic oligonucleotide
cagugagugauuaucaacccugcuuuugcu

SEQ ID NO: 89 - RNA, 30 nt, Artificial sequence
  Synthetic oligonucleotide
caaaaucaucaucucuuguuuuuguguguc

SEQ ID NO: 90 - RNA, 30 nt, Artificial sequence
  Synthetic oligonucleotide
auuuggauucauuuuaaucuccugcuuuug

SEQ ID NO: 91 - RNA, 30 nt, Artificial sequence
  Synthetic oligonucleotide
auuccaugcaaguuuuuucucuuguuuggu

SEQ ID NO: 92 - RNA, 30 nt, Artificial sequence
  Synthetic oligonucleotide
auuccauacauguuucuucucuuguuuggu

SEQ ID NO: 93 - RNA, 30 nt, Artificial sequence
  Synthetic oligonucleotide
auuccauacacguuuuuucucuugucuggu

SEQ ID NO: 94 - RNA, 30 nt, Artificial sequence
  Synthetic oligonucleotide
auuccaaacauguuucuucucuuguuuggu

SEQ ID NO: 95 - RNA, 30 nt, Artificial sequence
  Synthetic oligonucleotide
auuccaaacaaguuuuuccucuuguuuggu

SEQ ID NO: 96 - RNA, 30 nt, Artificial sequence
  Synthetic oligonucleotide
auuccaaacaaguuucuucucuuguuuggu

SEQ ID NO: 97 - RNA, 30 nt, Artificial sequence
  Synthetic oligonucleotide
augucaucuugaaaacgcuccgcuucugcu

SEQ ID NO: 98 - RNA, 30 nt, Artificial sequence
  Synthetic oligonucleotide
aucccauacauguuuuuucucuuguuuggu

SEQ ID NO: 99 - RNA, 30 nt, Artificial sequence
  Synthetic oligonucleotide
auccauucaagugguuugccugcuuuugcu

SEQ ID NO: 100 - RNA, 30 nt, Artificial sequence
  Synthetic oligonucleotide
auccauucaaaugguuugccugcuuuugcu

SEQ ID NO: 101 - RNA, 30 nt, Artificial sequence
  Synthetic oligonucleotide
auccauucaaaugguuugccugcuuucgcu

SEQ ID NO: 102 - RNA, 30 nt, Artificial sequence
  Synthetic oligonucleotide
auccauucaaaugguuucgcugcuuucgcu

SEQ ID NO: 103 - RNA, 30 nt, Artificial sequence
  Synthetic oligonucleotide
auaucaauuaguuuuuuuguuuuuucucgu

SEQ ID NO: 104 - RNA, 30 nt, Artificial sequence
  Synthetic oligonucleotide
accgauaucccaucuucauuuuccccuugg

SEQ ID NO: 105 - RNA, 30 nt, Artificial sequence
  Synthetic oligonucleotide
aaucacuauaguuuuuuuguuuuucuccgu

SEQ ID NO: 106 - RNA, 30 nt, Artificial sequence
  Synthetic oligonucleotide
aacacguauccauauuuccccuuguucggu

SEQ ID NO: 107 - RNA, 30 nt, Artificial sequence
  Synthetic oligonucleotide
aaaaucaucaucucuuguuuuugugugucu

SEQ ID NO: 108 - RNA, 26 nt, Artificial sequence
  Synthetic oligonucleotide
ucgacgucgauuuucggcgcgcgccg

SEQ ID NO: 109 - RNA, 26 nt, Artificial sequence
  Synthetic oligonucleotide
gcgauuucugaccgcuuuuuugucag

SEQ ID NO: 110 - RNA, 25 nt, Artificial sequence
  Synthetic oligonucleotide
guuguguuuuuacggcgccgugccg

SEQ ID NO: 111 - RNA, 21 nt, Artificial sequence
  Synthetic oligonucleotide
guuguguacggcgccgngccg

SEQ ID NO: 112 - RNA, 20 nt, Artificial sequence
  Synthetic oligonucleotide
uuuuucuuuuuguguguccg

SEQ ID NO: 113 - RNA, 20 nt, Artificial sequence
  Synthetic oligonucleotide
uuuuccccugcuuuugcuaa

SEQ ID NO: 114 - RNA, 20 nt, Artificial sequence
  Synthetic oligonucleotide
uuuaaucuccugcuuuugcu

SEQ ID NO: 115 - RNA, 20 nt, Artificial sequence
  Synthetic oligonucleotide
uuuaaacuccugcuuuugcu

SEQ ID NO: 116 - RNA, 20 nt, Artificial sequence
  Synthetic oligonucleotide
uuuaaaccccugcuuuugcu

SEQ ID NO: 117 - RNA, 20 nt, Artificial sequence
  Synthetic oligonucleotide
uuguacgcauuuuuucgcgu

SEQ ID NO: 118 - RNA, 20 nt, Artificial sequence
  Synthetic oligonucleotide
uuguacgcauuuuuucgcgu

SEQ ID NO: 119 - RNA, 20 nt, Artificial sequence
  Synthetic oligonucleotide
uuguacgcauuuuuucccgu

SEQ ID NO: 120 - RNA, 20 nt, Artificial sequence
  Synthetic oligonucleotide
uugguuuuguuguuaagcgu

SEQ ID NO: 121 - RNA, 20 nt, Artificial sequence
  Synthetic oligonucleotide
uugaucugguuguuaagcgu

SEQ ID NO: 122 - RNA, 20 nt, Artificial sequence
  Synthetic oligonucleotide
uugaucugguuguuaagcgu

SEQ ID NO: 123 - RNA, 20 nt, Artificial sequence
  Synthetic oligonucleotide
uucuuucuuuuugugugccc

SEQ ID NO: 124 - RNA, 20 nt, Artificial sequence
  Synthetic oligonucleotide
uuauuaacccugcuuuugcu

SEQ ID NO: 125 - RNA, 20 nt, Artificial sequence
  Synthetic oligonucleotide
uuaucuacccugcuuuugcu

SEQ ID NO: 126 - RNA, 20 nt, Artificial sequence
  Synthetic oligonucleotide
uuaucaacccugcuuuugcu

SEQ ID NO: 127 - RNA, 20 nt, Artificial sequence
  Synthetic oligonucleotide
uuacggauucucuguuuggu

SEQ ID NO: 128 - RNA, 20 nt, Artificial sequence
  Synthetic oligonucleotide
uuacagauucucuguuuggu

SEQ ID NO: 129 - RNA, 20 nt, Artificial sequence
  Synthetic oligonucleotide
uuaaaggcuccgcuucugcu

SEQ ID NO: 130 - RNA, 20 nt, Artificial sequence
  Synthetic oligonucleotide
uguuuuuucucuuguuuggu

SEQ ID NO: 131 - RNA, 20 nt, Artificial sequence
  Synthetic oligonucleotide
uguuuuuucucuuguuuggu

SEQ ID NO: 132 - RNA, 20 nt, Artificial sequence
  Synthetic oligonucleotide
uguuucuucucuuguuuggu

SEQ ID NO: 133 - RNA, 20 nt, Artificial sequence
  Synthetic oligonucleotide
ugaacuccugcuuuugcuaa

SEQ ID NO: 134 - RNA, 20 nt, Artificial sequence
  Synthetic oligonucleotide
ucuuucuuuuuguguguccg

SEQ ID NO: 135 - RNA, 20 nt, Artificial sequence
  Synthetic oligonucleotide
ucucuuguuuuugugugucu

SEQ ID NO: 136 - RNA, 20 nt, Artificial sequence
  Synthetic oligonucleotide
ucacggauucucuguuuggu

SEQ ID NO: 137 - RNA, 20 nt, Artificial sequence
  Synthetic oligonucleotide
ucaccgauucucuguuuggu

SEQ ID NO: 138 - RNA, 20 nt, Artificial sequence
  Synthetic oligonucleotide
ucacagauucucuguuuggu

SEQ ID NO: 139 - RNA, 20 nt, Artificial sequence
  Synthetic oligonucleotide
ucaaguuacuuuuuuuauac

SEQ ID NO: 140 - RNA, 20 nt, Artificial sequence
  Synthetic oligonucleotide
ucaaguuacuuuucuuauac

SEQ ID NO: 141 - RNA, 20 nt, Artificial sequence
  Synthetic oligonucleotide
ucaaacguaucgcuucugcu

SEQ ID NO: 142 - RNA, 20 nt, Artificial sequence
  Synthetic oligonucleotide
ucaaacgcaccgcuucugcu

SEQ ID NO: 143 - RNA, 20 nt, Artificial sequence
  Synthetic oligonucleotide
uauuuuccccugcuuuugcu

SEQ ID NO: 144 - RNA, 20 nt, Artificial sequence
  Synthetic oligonucleotide
uauacgcguuuuuuucgcgu

SEQ ID NO: 145 - RNA, 20 nt, Artificial sequence
  Synthetic oligonucleotide
uaguacgcauuuuuucgcgu

SEQ ID NO: 146 - RNA, 20 nt, Artificial sequence
  Synthetic oligonucleotide
uaguacgcauuuuuucgcgu

SEQ ID NO: 147 - RNA, 20 nt, Artificial sequence
  Synthetic oligonucleotide
guuuuuuuguuuuuucucgu

SEQ ID NO: 148 - RNA, 20 nt, Artificial sequence
  Synthetic oligonucleotide
guuuuuuuguuuuucuccgu

SEQ ID NO: 149 - RNA, 20 nt, Artificial sequence
  Synthetic oligonucleotide
gugguuugccugcuuuugcu

SEQ ID NO: 150 - RNA, 20 nt, Artificial sequence
  Synthetic oligonucleotide
gauuucccccugcuuuugcu

SEQ ID NO: 151 - RNA, 20 nt, Artificial sequence
  Synthetic oligonucleotide
gaucaguaccugcuuuugcu

SEQ ID NO: 152 - RNA, 20 nt, Artificial sequence
  Synthetic oligonucleotide
gaucaguaccugcuuucgcu

SEQ ID NO: 153 - RNA, 20 nt, Artificial sequence
  Synthetic oligonucleotide
gaaaaugcucugcuucugcu

SEQ ID NO: 154 - RNA, 20 nt, Artificial sequence
  Synthetic oligonucleotide
gaaaauagccaaucuuagcu

SEQ ID NO: 155 - RNA, 20 nt, Artificial sequence
  Synthetic oligonucleotide
gaaaacgcuccgcuucugcu

SEQ ID NO: 156 - RNA, 20 nt, Artificial sequence
  Synthetic oligonucleotide
cuuagccaacuuugucuggu

SEQ ID NO: 157 - RNA, 20 nt, Artificial sequence
  Synthetic oligonucleotide
cuuacccagcuuuguuuggu

SEQ ID NO: 158 - RNA, 20 nt, Artificial sequence
  Synthetic oligonucleotide
cuuacccagcuuugucuggu

SEQ ID NO: 159 - RNA, 20 nt, Artificial sequence
  Synthetic oligonucleotide
cuuacccaacuuuguuuggu

SEQ ID NO: 160 - RNA, 20 nt, Artificial sequence
  Synthetic oligonucleotide
cuuacccaacuuuguuuggu

SEQ ID NO: 161 - RNA, 20 nt, Artificial sequence
  Synthetic oligonucleotide
cuuaaagcuccgcuucugcu

SEQ ID NO: 162 - RNA, 20 nt, Artificial sequence
  Synthetic oligonucleotide
cugguuuuguuguuaagcgu

SEQ ID NO: 163 - RNA, 20 nt, Artificial sequence
  Synthetic oligonucleotide
cucuuaaacucuugucuggu

SEQ ID NO: 164 - RNA, 20 nt, Artificial sequence
  Synthetic oligonucleotide
cucaucaucuuuuaugauac

SEQ ID NO: 165 - RNA, 20 nt, Artificial sequence
  Synthetic oligonucleotide
cguuuuuucucuugucuggu

SEQ ID NO: 166 - RNA, 20 nt, Artificial sequence
  Synthetic oligonucleotide
caucuucauuuuccccuugg

SEQ ID NO: 167 - RNA, 20 nt, Artificial sequence
  Synthetic oligonucleotide
cauauuuccccuuguucggu

SEQ ID NO: 168 - RNA, 20 nt, Artificial sequence
  Synthetic oligonucleotide
cauagccaacuuuuucuggu

SEQ ID NO: 169 - RNA, 20 nt, Artificial sequence
  Synthetic oligonucleotide
auuuuccccugcuuuugcua

SEQ ID NO: 170 - RNA, 20 nt, Artificial sequence
  Synthetic oligonucleotide
auuuuaaucuccugcuuuug

SEQ ID NO: 171 - RNA, 20 nt, Artificial sequence
  Synthetic oligonucleotide
auugguuuuuuugucuucgu

SEQ ID NO: 172 - RNA, 20 nt, Artificial sequence
  Synthetic oligonucleotide
auugguuuguuuuucuucgu

SEQ ID NO: 173 - RNA, 20 nt, Artificial sequence
  Synthetic oligonucleotide
augguuuuuuuguuaagcgu

SEQ ID NO: 174 - RNA, 20 nt, Artificial sequence
  Synthetic oligonucleotide
augguuugccugcuuuugcu

SEQ ID NO: 175 - RNA, 20 nt, Artificial sequence
  Synthetic oligonucleotide
augguuugccugcuuucgcu

SEQ ID NO: 176 - RNA, 20 nt, Artificial sequence
  Synthetic oligonucleotide
augguuucgcugcuuucgcu

SEQ ID NO: 177 - RNA, 20 nt, Artificial sequence
  Synthetic oligonucleotide
aucuucuuuuuccccuuggu

SEQ ID NO: 178 - RNA, 20 nt, Artificial sequence
  Synthetic oligonucleotide
aucuucauuuuccccuuggu

SEQ ID NO: 179 - RNA, 20 nt, Artificial sequence
  Synthetic oligonucleotide
aucucuuguuuuuguguguc

SEQ ID NO: 180 - RNA, 20 nt, Artificial sequence
  Synthetic oligonucleotide
aucaucaucuuuuuugauac

SEQ ID NO: 181 - RNA, 20 nt, Artificial sequence
  Synthetic oligonucleotide
auauucauucuccccuuggu

SEQ ID NO: 182 - RNA, 20 nt, Artificial sequence
  Synthetic oligonucleotide
auaauugcgcugcuuucgcu

SEQ ID NO: 183 - RNA, 20 nt, Artificial sequence
  Synthetic oligonucleotide
auaauugaccugcuuuucgu

SEQ ID NO: 184 - RNA, 20 nt, Artificial sequence
  Synthetic oligonucleotide
auaauugaccugcuuucgcu

SEQ ID NO: 185 - RNA, 20 nt, Artificial sequence
  Synthetic oligonucleotide
auaauugaccugcuuucgcu

SEQ ID NO: 186 - RNA, 20 nt, Artificial sequence
  Synthetic oligonucleotide
aguuuuuucucuuguuuggu

SEQ ID NO: 187 - RNA, 20 nt, Artificial sequence
  Synthetic oligonucleotide
aguuuuuccucuuguuuggu

SEQ ID NO: 188 - RNA, 20 nt, Artificial sequence
  Synthetic oligonucleotide
aguuucuucucuuguuuggu

SEQ ID NO: 189 - RNA, 20 nt, Artificial sequence
  Synthetic oligonucleotide
aguuucuucucuuguuuggu

SEQ ID NO: 190 - RNA, 20 nt, Artificial sequence
  Synthetic oligonucleotide
agucucuucucuuguuuggu

SEQ ID NO: 191 - RNA, 20 nt, Artificial sequence
  Synthetic oligonucleotide
aauuuuccccugcuuuugcu

SEQ ID NO: 192 - RNA, 20 nt, Artificial sequence
  Synthetic oligonucleotide
aaugguuuuuuugucuucgu

SEQ ID NO: 193 - RNA, 20 nt, Artificial sequence
  Synthetic oligonucleotide
aaugguuuguuugucuucgu

SEQ ID NO: 194 - RNA, 20 nt, Artificial sequence
  Synthetic oligonucleotide
aaugguuuauuugucuucgu

SEQ ID NO: 195 - RNA, 20 nt, Artificial sequence
  Synthetic oligonucleotide
aaucaguaccugcuuuugcu

SEQ ID NO: 196 - RNA, 20 nt, Artificial sequence
  Synthetic oligonucleotide
aaucaguaccugcuuucgcu

SEQ ID NO: 197 - RNA, 20 nt, Artificial sequence
  Synthetic oligonucleotide
aauaucuaccugcuuuugcu

SEQ ID NO: 198 - RNA, 20 nt, Artificial sequence
  Synthetic oligonucleotide
aauaucuaccugcuuucgcu

SEQ ID NO: 199 - RNA, 20 nt, Artificial sequence
  Synthetic oligonucleotide
aacaucuaccugcuuuugcu

SEQ ID NO: 200 - RNA, 18 nt, Artificial sequence
  Synthetic oligonucleotide
uuuuuucggcggccgccg

SEQ ID NO: 201 - RNA, 18 nt, Artificial sequence
  Synthetic oligonucleotide
cggcggccgccguuuuuu

SEQ ID NO: 202 - RNA, 18 nt, Artificial sequence
  Synthetic oligonucleotide
ccgucuguuguuggacuc

SEQ ID NO: 203 - RNA, 18 nt, Artificial sequence
  Synthetic oligonucleotide
ccgucuguugugugacag

SEQ ID NO: 204 - RNA, 18 nt, Artificial sequence
  Synthetic oligonucleotide
ggggggguuguguggggg

SEQ ID NO: 205 - RNA, 18 nt, Artificial sequence
  Synthetic oligonucleotide
cgacucucucuucaguug

SEQ ID NO: 206 - RNA, 18 nt, Artificial sequence
  Synthetic oligonucleotide
ccgucuguugugugacuc

SEQ ID NO: 207 - RNA, 16 nt, Artificial sequence
  Synthetic oligonucleotide
uuuucggcggccgccg

SEQ ID NO: 208 - RNA, 16 nt, Artificial sequence
  Synthetic oligonucleotide
cggcggccgccguuuu

SEQ ID NO: 209 - RNA, 16 nt, Artificial sequence
  Synthetic oligonucleotide
uuuucggcgcgcgccg

SEQ ID NO: 210 - RNA, 16 nt, Artificial sequence
  Synthetic oligonucleotide
cggcgcgcgccguuuu

SEQ ID NO: 211 - RNA, 15 nt, Artificial sequence
  Synthetic oligonucleotide
uuuuuuugucuucgu

SEQ ID NO: 212 - RNA, 15 nt, Artificial sequence
  Synthetic oligonucleotide
uuuuuuguuaagcgu

SEQ ID NO: 213 - RNA, 15 nt, Artificial sequence
  Synthetic oligonucleotide
uuuguuuuuucucgu

SEQ ID NO: 214 - RNA, 15 nt, Artificial sequence
  Synthetic oligonucleotide
uuuguuuuucuucgu

SEQ ID NO: 215 - RNA, 15 nt, Artificial sequence
  Synthetic oligonucleotide
uuuguuuuucuccgu

SEQ ID NO: 216 - RNA, 15 nt, Artificial sequence
  Synthetic oligonucleotide
uuuguuugucuucgu

SEQ ID NO: 217 - RNA, 15 nt, Artificial sequence
  Synthetic oligonucleotide
uuuguuguuaagcgu

SEQ ID NO: 218 - RNA, 15 nt, Artificial sequence
  Synthetic oligonucleotide
uuucucuuguuuggu

SEQ ID NO: 219 - RNA, 15 nt, Artificial sequence
  Synthetic oligonucleotide
uuucucuugucuggu

SEQ ID NO: 220 - RNA, 15 nt, Artificial sequence
  Synthetic oligonucleotide
uuuauuugucuucgu

SEQ ID NO: 221 - RNA, 15 nt, Artificial sequence
  Synthetic oligonucleotide
uuguuuuuguguguc

SEQ ID NO: 222 - RNA, 15 nt, Artificial sequence
  Synthetic oligonucleotide
uugccugcuuuugcu

SEQ ID NO: 223 - RNA, 15 nt, Artificial sequence
  Synthetic oligonucleotide
uugccugcuuucgcu

SEQ ID NO: 224 - RNA, 15 nt, Artificial sequence
  Synthetic oligonucleotide
uucgcugcuuucgcu

SEQ ID NO: 225 - RNA, 15 nt, Artificial sequence
  Synthetic oligonucleotide
uuccucuuguuuggu

SEQ ID NO: 226 - RNA, 15 nt, Artificial sequence
  Synthetic oligonucleotide
uuccccuuguucggu

SEQ ID NO: 227 - RNA, 15 nt, Artificial sequence
  Synthetic oligonucleotide
uuacuuuuuuuauac

SEQ ID NO: 228 - RNA, 15 nt, Artificial sequence
  Synthetic oligonucleotide
uuacuuuucuuauac

SEQ ID NO: 229 - RNA, 15 nt, Artificial sequence
  Synthetic oligonucleotide
uguuuuugugugucu

SEQ ID NO: 230 - RNA, 15 nt, Artificial sequence
  Synthetic oligonucleotide
ugcucugcuucugcu

SEQ ID NO: 231 - RNA, 15 nt, Artificial sequence
  Synthetic oligonucleotide
ugcgcugcuuucgcu

SEQ ID NO: 232 - RNA, 15 nt, Artificial sequence
  Synthetic oligonucleotide
ugaccugcuuuucgu

SEQ ID NO: 233 - RNA, 15 nt, Artificial sequence
  Synthetic oligonucleotide
ugaccugcuuucgcu

SEQ ID NO: 234 - RNA, 15 nt, Artificial sequence
  Synthetic oligonucleotide
ucuuuuugugugccc

SEQ ID NO: 235 - RNA, 15 nt, Artificial sequence
  Synthetic oligonucleotide
ucuccugcuuuugcu

SEQ ID NO: 236 - RNA, 15 nt, Artificial sequence
  Synthetic oligonucleotide
uccugcuuuugcuaa

SEQ ID NO: 237 - RNA, 15 nt, Artificial sequence
  Synthetic oligonucleotide
uccccugcuuuugcu

SEQ ID NO: 238 - RNA, 15 nt, Artificial sequence
  Synthetic oligonucleotide
ucauuuuccccuugg

SEQ ID NO: 239 - RNA, 15 nt, Artificial sequence
  Synthetic oligonucleotide
uagccaaucuuagcu

SEQ ID NO: 240 - RNA, 15 nt, Artificial sequence
  Synthetic oligonucleotide
uacccugcuuuugcu

SEQ ID NO: 241 - RNA, 15 nt, Artificial sequence
  Synthetic oligonucleotide
guaccugcuuuugcu

SEQ ID NO: 242 - RNA, 15 nt, Artificial sequence
  Synthetic oligonucleotide
guaccugcuuucgcu

SEQ ID NO: 243 - RNA, 15 nt, Artificial sequence
  Synthetic oligonucleotide
ggcuccgcuucugcu

SEQ ID NO: 244 - RNA, 15 nt, Artificial sequence
  Synthetic oligonucleotide
gcguuuuuuucgcgu

SEQ ID NO: 245 - RNA, 15 nt, Artificial sequence
  Synthetic oligonucleotide
gauucucuguuuggu

SEQ ID NO: 246 - RNA, 15 nt, Artificial sequence
  Synthetic oligonucleotide
cuuuuuguguguccg

SEQ ID NO: 247 - RNA, 15 nt, Artificial sequence
  Synthetic oligonucleotide
cuuuuuccccuuggu

SEQ ID NO: 248 - RNA, 15 nt, Artificial sequence
  Synthetic oligonucleotide
cuucucuuguuuggu

SEQ ID NO: 249 - RNA, 15 nt, Artificial sequence
  Synthetic oligonucleotide
cugguuguuaagcgu

SEQ ID NO: 250 - RNA, 15 nt, Artificial sequence
  Synthetic oligonucleotide
cuaccugcuuuugcu

SEQ ID NO: 251 - RNA, 15 nt, Artificial sequence
  Synthetic oligonucleotide
cuaccugcuuucgcu

SEQ ID NO: 252 - RNA, 15 nt, Artificial sequence
  Synthetic oligonucleotide
cguaucgcuucugcu

SEQ ID NO: 253 - RNA, 15 nt, Artificial sequence
  Synthetic oligonucleotide
cgcuccgcuucugcu

SEQ ID NO: 254 - RNA, 15 nt, Artificial sequence
  Synthetic oligonucleotide
cgcauuuuuucgcgu

SEQ ID NO: 255 - RNA, 15 nt, Artificial sequence
  Synthetic oligonucleotide
cgcauuuuuucccgu

SEQ ID NO: 256 - RNA, 15 nt, Artificial sequence
  Synthetic oligonucleotide
cgcaccgcuucugcu

SEQ ID NO: 257 - RNA, 15 nt, Artificial sequence
  Synthetic oligonucleotide
cccugcuuuugcuaa

SEQ ID NO: 258 - RNA, 15 nt, Artificial sequence
  Synthetic oligonucleotide
ccccugcuuuugcua

SEQ ID NO: 259 - RNA, 15 nt, Artificial sequence
  Synthetic oligonucleotide
ccagcuuuguuuggu

SEQ ID NO: 260 - RNA, 15 nt, Artificial sequence
  Synthetic oligonucleotide
ccagcuuugucuggu

SEQ ID NO: 261 - RNA, 15 nt, Artificial sequence
  Synthetic oligonucleotide
ccaacuuuuucuggu

SEQ ID NO: 262 - RNA, 15 nt, Artificial sequence
  Synthetic oligonucleotide
ccaacuuuguuuggu

SEQ ID NO: 263 - RNA, 15 nt, Artificial sequence
  Synthetic oligonucleotide
ccaacuuugucuggu

SEQ ID NO: 264 - RNA, 15 nt, Artificial sequence
  Synthetic oligonucleotide
cauuuuccccuuggu

SEQ ID NO: 265 - RNA, 15 nt, Artificial sequence
  Synthetic oligonucleotide
cauucuccccuuggu

SEQ ID NO: 266 - RNA, 15 nt, Artificial sequence
  Synthetic oligonucleotide
caucuuuuuugauac

SEQ ID NO: 267 - RNA, 15 nt, Artificial sequence
  Synthetic oligonucleotide
caucuuuuaugauac

SEQ ID NO: 268 - RNA, 15 nt, Artificial sequence
  Synthetic oligonucleotide
cacccugcuuuugcu

SEQ ID NO: 269 - RNA, 15 nt, Artificial sequence
  Synthetic oligonucleotide
agcuccgcuucugcu

SEQ ID NO: 270 - RNA, 15 nt, Artificial sequence
  Synthetic oligonucleotide
acuccugcuuuugcu

SEQ ID NO: 271 - RNA, 15 nt, Artificial sequence
  Synthetic oligonucleotide
accccugcuuuugcu

SEQ ID NO: 272 - RNA, 15 nt, Artificial sequence
  Synthetic oligonucleotide
aaucuccugcuuuug

SEQ ID NO: 273 - RNA, 15 nt, Artificial sequence
  Synthetic oligonucleotide
aacccugcuuuugcu

SEQ ID NO: 274 - RNA, 15 nt, Artificial sequence
  Synthetic oligonucleotide
aaacucuugucuggu

SEQ ID NO: 275 - RNA, 14 nt, Artificial sequence
  Synthetic oligonucleotide
ucgacgucgauuuu

SEQ ID NO: 276 - RNA, 14 nt, Artificial sequence
  Synthetic oligonucleotide
cuguugugugacag

SEQ ID NO: 277 - RNA, 13 nt, Artificial sequence
  Synthetic oligonucleotide
gggguuuuggggg

SEQ ID NO: 278 - RNA, 13 nt, Artificial sequence
  Synthetic oligonucleotide
ccccuuuuggggg

SEQ ID NO: 279 - RNA, 13 nt, Artificial sequence
  Synthetic oligonucleotide
ggggggguugugu

SEQ ID NO: 280 - RNA, 12 nt, Artificial sequence
  Synthetic oligonucleotide
guuugugugggg

SEQ ID NO: 281 - RNA, 12 nt, Artificial sequence
  Synthetic oligonucleotide
gggguuuugggg

SEQ ID NO: 282 - RNA, 12 nt, Artificial sequence
  Synthetic oligonucleotide
gggguuuucccc

SEQ ID NO: 283 - RNA, 12 nt, Artificial sequence
  Synthetic oligonucleotide
cggcuuuugccg

SEQ ID NO: 284 - RNA, 12 nt, Artificial sequence
  Synthetic oligonucleotide
gaucuuuugauc

SEQ ID NO: 285 - RNA, 12 nt, Artificial sequence
  Synthetic oligonucleotide
guuguguggggg

SEQ ID NO: 286 - RNA, 12 nt, Artificial sequence
  Synthetic oligonucleotide
cuucggcuucgg

SEQ ID NO: 287 - RNA, 12 nt, Artificial sequence
  Synthetic oligonucleotide
ggacuuuggucc

SEQ ID NO: 288 - RNA, 12 nt, Artificial sequence
  Synthetic oligonucleotide
gucggcguugac

SEQ ID NO: 289 - RNA, 12 nt, Artificial sequence
  Synthetic oligonucleotide
gaucuuuucggc

SEQ ID NO: 290 - RNA, 12 nt, Artificial sequence
  Synthetic oligonucleotide
auauuuuucggc

SEQ ID NO: 291 - RNA, 11 nt, Artificial sequence
  Synthetic oligonucleotide
uuuuuuggggg

SEQ ID NO: 292 - RNA, 11 nt, Artificial sequence
  Synthetic oligonucleotide
uuuuuuggggg

SEQ ID NO: 293 - RNA, 10 nt, Artificial sequence
  Synthetic oligonucleotide
uuuuuuauac

SEQ ID NO: 294 - RNA, 10 nt, Artificial sequence
  Synthetic oligonucleotide
uuuuugauac

SEQ ID NO: 295 - RNA, 10 nt, Artificial sequence
  Synthetic oligonucleotide
uuuuucuggu

SEQ ID NO: 296 - RNA, 10 nt, Artificial sequence
  Synthetic oligonucleotide
uuuuucucgu

SEQ ID NO: 297 - RNA, 10 nt, Artificial sequence
  Synthetic oligonucleotide
uuuuucgcgu

SEQ ID NO: 298 - RNA, 10 nt, Artificial sequence
  Synthetic oligonucleotide
uuuuucccgu

SEQ ID NO: 299 - RNA, 10 nt, Artificial sequence
  Synthetic oligonucleotide
uuuucuucgu

SEQ ID NO: 300 - RNA, 10 nt, Artificial sequence
  Synthetic oligonucleotide
uuuucuccgu

SEQ ID NO: 301 - RNA, 10 nt, Artificial sequence
  Synthetic oligonucleotide
uuuguuuggu

SEQ ID NO: 302 - RNA, 10 nt, Artificial sequence
  Synthetic oligonucleotide
uuuguguguc

SEQ ID NO: 303 - RNA, 10 nt, Artificial sequence
  Synthetic oligonucleotide
uuugucuggu

SEQ ID NO: 304 - RNA, 10 nt, Artificial sequence
  Synthetic oligonucleotide
uuucuuauac

SEQ ID NO: 305 - RNA, 10 nt, Artificial sequence
  Synthetic oligonucleotide
uuuaugauac

SEQ ID NO: 306 - RNA, 10 nt, Artificial sequence
  Synthetic oligonucleotide
uugugugucu

SEQ ID NO: 307 - RNA, 10 nt, Artificial sequence
  Synthetic oligonucleotide
uugugugccc

SEQ ID NO: 308 - RNA, 10 nt, Artificial sequence
  Synthetic oligonucleotide
uugucuucgu

SEQ ID NO: 309 - RNA, 10 nt, Artificial sequence
  Synthetic oligonucleotide
uuccccuugg

SEQ ID NO: 310 - RNA, 10 nt, Artificial sequence
  Synthetic oligonucleotide
uguuaagcgu

SEQ ID NO: 311 - RNA, 10 nt, Artificial sequence
  Synthetic oligonucleotide
uguguguccg

SEQ ID NO: 312 - RNA, 10 nt, Artificial sequence
  Synthetic oligonucleotide
ugcuuuugcu

SEQ ID NO: 313 - RNA, 10 nt, Artificial sequence
  Synthetic oligonucleotide
ugcuuuucgu

SEQ ID NO: 314 - RNA, 10 nt, Artificial sequence
  Synthetic oligonucleotide
ugcuuucgcu

SEQ ID NO: 315 - RNA, 10 nt, Artificial sequence
  Synthetic oligonucleotide
ugcuucugcu

SEQ ID NO: 316 - RNA, 10 nt, Artificial sequence
  Synthetic oligonucleotide
ucuguuuggu

SEQ ID NO: 317 - RNA, 10 nt, Artificial sequence
  Synthetic oligonucleotide
uccccuuggu

SEQ ID NO: 318 - RNA, 10 nt, Artificial sequence
  Synthetic oligonucleotide
gcuuuugcua

SEQ ID NO: 319 - RNA, 10 nt, Artificial sequence
  Synthetic oligonucleotide
cuuuugcuaa

SEQ ID NO: 320 - RNA, 10 nt, Artificial sequence
  Synthetic oligonucleotide
cuuguuuggu

SEQ ID NO: 321 - RNA, 10 nt, Artificial sequence
  Synthetic oligonucleotide
cuuguucggu

SEQ ID NO: 322 - RNA, 10 nt, Artificial sequence
  Synthetic oligonucleotide
cuugucuggu

SEQ ID NO: 323 - RNA, 10 nt, Artificial sequence
  Synthetic oligonucleotide
cgcuucugcu

SEQ ID NO: 324 - RNA, 10 nt, Artificial sequence
  Synthetic oligonucleotide
ccugcuuuug

SEQ ID NO: 325 - RNA, 10 nt, Artificial sequence
  Synthetic oligonucleotide
aaucuuagcu

SEQ ID NO: 326 - RNA, 10 nt, Artificial sequence
  Synthetic oligonucleotide
uuuuuuuuuu

SEQ ID NO: 327 - RNA, 10 nt, Artificial sequence
  Synthetic oligonucleotide
uuuuuggggg

SEQ ID NO: 328 - RNA, 10 nt, Artificial sequence
  Synthetic oligonucleotide
ggggguuuuu

SEQ ID NO: 329 - RNA, 10 nt, Artificial sequence
  Synthetic oligonucleotide
uuguggguca

SEQ ID NO: 330 - RNA, 16 nt, Artificial sequence
  Synthetic oligonucleotide
cgcgcggcgcgcgccg

SEQ ID NO: 331 - RNA, 20 nt, Artificial sequence
  Synthetic oligonucleotide
uuguuguuguuguuguuguu